SEQUENCE LISTING (17 sequences)

SEQ ID NO: 1 - DNA, 20840 bp, Homo sapiens
  misc_feature (4951)..(5026): exon 1
tggcacaatctcggctcactacaacctttgcctcctgggttcaagtgattctcctgcctcagcctcccaagtagctgggattacaagtgcgtgccaccacacctggccaattttgtatttttagtagagatggggtttcaccatgttggccgggctggtctccaactcctgacctcagctgatctgcctgcctcagcctcccaaagtgctgtaattataggcataagccactgtgcctggcctagtgttgtacattctgtgggttttgacaattgtatgcatctacatgtatgtaccattatagtattcctgtttttaattttagccattctagtaggcatgtagtgatatctcatggtgattttaatttgcgtttccgtaatggttaataatgctgaacatctttgcatgtgcttgtttgtcatttgtgtttcctacttggtgaaataattgttcatgtcctttgtccattttctaattgaatttttttttaccatttagttttgagatttctttatacaatctagatccaaatctcttgtctcaaatatggtttgcaaatacattcctctaattcatatattgccttttcctcctcttaacaggatgtttcacagagcaaaagttttagttttgttgaaatctcacttttcatttttttctttagtggattgtgcttttgttgtcatatgtaagaactcttcactggccctagatccttgtattggtttcctaagattgccatagcaaatcaccatgaacttagtgacaaaaagacagaaatttattttcacttcctactgtgggcagactagacgttaattattttcatgtatgctcattcctatgacatctttctgatataataattatagttattcttaagcttcacccttttttctattagctttgttaccttgggtgtcactttttcttttttgacattgtgacctatgccagatcatgtctgttagtacttagccctccattcacctctccataatcccttttgtattcctggagcttgatgcctgaaatgacacatcctacattcctttgccagatgggtaccagttagcttgtgcacatgggagacaaccgtgaaaagactgaagtggggaagaagggaggagctgttgtgtttcagtgagcgcccttggcagtggcggtgacagtggctcctgttcagtggcaatggtggagcagctagcaagacatgcagtaagcgcaggctcataggctatggtccaggagcagtcaccgattcctggtctttaggcaatatcatctccctttgcttctccagcctttctaaaattattgtaccttgactagtacaattttttagtattgggggtagtccaaggacacaggctttaaaaagtatgaattcagggttgcctacctgcattgactgcgcttgaatcatgatggccttctggtcggtggcaggaggtgacagtccaaatcatgcagtagcaaaccagatacttaaattatcatctgagatacttcagaagtacagccgtagccataccttcagaagagataaagaaatgttctcctggccaggcgcggtggctcacgcctgtcattccagcactttgggaggccgagggggtggatcacctgaggtcgggagttcgagaccagcctgaccaacatggggaaaccctgtctctactaaaaatacaaaattagcggggcgtggtggcacatgcccataatcccagctactcgggaggctaaggcaggataatcgcttgaacctgagaggcagaggttgcggtgaactgagatcatgccatagtactccagcctgggcaacaagagtgaaactccatctcaaaaaaaaaaaaaaagaaaaaaagataaagaaatgttctcctttcttgccatttctaggggtttggggatggcgtacattgctgcagggcgtgctcactctaccatcttgctccaatctttatttttcaaaatacagtgcttatgcttggttacttcagttaagattatttttaaaaatcataattaagcaaaaatatatggccatgcttaaacatatttaagataaattaagtgatttggcctgtttcagtatcccaactcacatgctaacaggggcttgacctgtagctacggtaccctggaggaaatgatcgcatttatttggttatttcggtctaagtagtaatagttctgtcctgggaaaaagactagcctcaaggcatttctgattgaatgtttttcaattacagtctttaaaccagtatgccacagaactggctctttccacatgacggcctttgtggtgggtggcagattgccctgaggcctcgcaaaatgctaggctttcacaatgtcactgactgacagccaggcccagcacagtcttggtgtgattgtggggctaaagttattccaccttgtgcaatagctacagctttctctaaccagctgcattcttataaagttagaagaaaatacttttttttttttgagatggattctcgctctgttgcccaggctggagtgcaatggtgcgatctcggctcgctgcaacctccgcctcctgggttcaaacgattctcctccctcagacccccgagtagctgggattgcaggtgcctgccaccacgcccggctaacttttttgtatttttagtggagacggggtttcaccatcttcgtcaggctggtctcagactcctgacctcaagtgatctgcccgcctcagcctcccaaaatgctgggattacaggcatgagctactgtgcccggccaaagaaaatactttttatgccagccctgaaactaccctgaagcacatacatcaaccttgaggcctcacactccatcaagaggggtgaagggcatgaggaattagaaagcatagggatttttagttagacagatctggttcaaatcctagacttgtgccttgaacaaattatttaccctcattgaactctagattcattatttgtaaaatgaaagacaataatagttatctccaaaggaaagttgaatatgatcattcatttattcattaattcaacatttattattgcctactttgtgccaggttctattctaggaactaagggatacaactttgaataggcaaaatctctgctctcctgaagtttacttttttttttttttttgagacagagtttcactcttgtcacccaggctagagcgcaatggtgctcttggctcactgcaacctccacctcctgggttcaagtgattctcttgtctcagcctcccaagtagctgggactacaggtatgtgccaccacgcccggctatttctgcatttttagtagagatggggtttcaccatgttggccagactggtctcaaactcctgatctcaggtgatatgcctgtcttggccttccaaagtactgggattacaggcctgagccactgcacctgacctgaagtttatgttctattaaatagcaacagacagtaacataaaccaaaaataaataggaaaacaccataacaaaaatcaaacagtgatataattgagagttgcttctatttctttttgttgtcttcttggttcaatcagcctgctaaactatatggaacctcattttcatgggccacttatttaagccgggggaccttggaaagtctctcatgtctctcatctcaacggcctaatgtgacttctcttgaaatatttggacattagcaggaagctgaggctttacatcagatctttactttaatggtggacttgactttactggtagatttttaggctctgtgtggactgtggagatgatatctggggggcaggcagacacttgccctgcctctgtctgagaaaattctgttttggatgtcttgttgaagttggtgctggcatcctaagcccttgctggggtcgtaatttaattcatcagaatgtgtggcttgcaagaaccggctcagatcctgcccttcaaaaacaaaacatgagcgtgccaagaaagtccaaggtgttgaatgttgccacttcaagcctaaactttctaggaacacctaagtgggtggcagcttccagttctccaggctgcttctaggccagagctgggttccacaagagacagaataggcatatatatgcttaaggaactggaaaaacaggctctctctctctcacaaacacacacacacacataccaaggtagctgtcaaaatgttatccgaaattttggaaccaaaaaatcttgaaagatggtattccaatatcacattttatgtaagttttctattatattagattcaaattacgattcgaggccacaagctttaagaattcagggcctttttaacttgccaagccccacaccactccaggaacttccccacaccccagttctcagaattcatgtgcaaggtctttcctaaatccagggtccaggtcagagagtggaggatgtgctctatttcttacctgattgcagacccctctgacagtgctcccttctgaagcactcactgtctgaacgtacacagtctcagacttaatcatgcacagtgagcaagactgtggtgtgataattggcgtccctgacttattagggcaaatctatgggagggggagacctcctggaccactgagcaattaattcatttacattaggaagtttctccgtcagatgcaggaaaaaaatcttgttttcctgctgtggttttgacttttgccccatcttctgttgctgttgtaggaggcaaaataagggtcaaggcctggaaacacaagtgctttgactgaagctccacttggcttccgaagcccaagctgggttgtaccaggttccctagggtgcaggctgtgggcaactgccagggacatgtgcctgcccaccggcctctggccctcactgagttggccaatgggaaatgacaattgtgaggtggggactgcctgcccccgtgagtaccaggctgttgaggctgggccatctcctcctcacttccaytctgactgcagtctgtggttctgattccataccagagggtaagagcaattctgtgaagttccaggctgggtgggggatgcatgcatagcctctggctgggatcacccaggctctcccgtccgtagtagtgtgggagtggatacaggtggatactctggtcagagcagcactggtggaggcagatatgcactgggcttcttcctccgttctcccacagccccaagagagaaagggttatttcagacattccttctaagatgcatggaaccattctgaattttgcccagttcgctctgtagcaggatacctattgagaaaaagttagggtcagtaaggtggaagggtctgtccacagatgaagtccaattcgattaagggggataagggaatacattgcctcttagcttgaccaggtagggcaaaggaagaagcatatatgaaggcagcttcagaaaagtcaagctgagcactgacttcagactggaattaggaatccagctctgccactttattctactcagcaaatatttactgagcaaattctatgggctagacagtggattgggttcacaagatacaatgagtgtgacatggttgttgtctatggatttggggatatatgtaggtatagggatatcttacaaggtaatcaagaggttctaatgaggccagccatggtggctcacacctgtaatcccagcaatttgggagaccgaggcgggtggatcacctgaggtcaggagttccagactagcctgaccaacatggtgaaaccccgcctctaccaaaaatacaaaaattagttgggcgtgatggcaggtgcctgtaatcccagcttctcgggaggctgaggcaggagaattgtctgaacctgggaggcagaggttgcagtgagccgagattgttgccactgcattccagcctgggtgacagagcgagactttgtgtcaaaaaaaaaaaaaaaagaaagaaaagaaaaagaggctctaatgagataaaatgagaaaagcctggcatatagtggcaacttatgaaaaattgtaattaaaaaaaaacattttctgacagaagaaactggatctacctggtttttctgaagcctaatcctgctcgccccagtgagtgctgtttctgaggcatcctggttgttttgagctgtggatgctgaaggttagagtgggagggattttagaggttaggtctgcccctcttgtgttagaggacatggatccctggtctggagaggttctggtttttggatcaagcctcacaaggggtggcaccaactcactcctaggaactccgctagaaggaaggccagctctgcctaattcggttggggagatgggggtccctttatgctagcagaatatgtccgaaggagcatgatggtgtcagctttgttcatgaaggccagtggtacacagggagcccggcagcttcctcagcagtccctgctgccactcttccttaagtcttgaggagtctttttttggcacaatctcagctcactgcaacctccgcctcccaggttcaagcgattctcctgcctcagtctcccaagtagctgagactacaggcatgcgccaccacgcccagctaatttttatatttttagtagagatggggttcaccatattggccaggatggtctcgatctcttgacctcatattccacctgcctcggcctcccaaagtgctggtattacaggtgtgagccactgcgcctggccgaggagtcttaagctgagatcacagcattgcactccagcctgggcaaaaaaagcaaaactccatctcaaaaaaaaaaaaaaaaaaatagacacaagactggctccttgtcttttttggggacagggtctcactctatcacccaggctggagtgcagtggtgcaatcacagctcactgcagcctcgatttcccaggctcaagtgaccctcccatcttagcctcctgagtagctgggactacaggtgtgtgcaaccatgcctggctaatttttaaaaattttttgtagagatgaggtctcactatattggctggggggcctcaaactcctgggctcagcagtcctcccacctcagcctcccaaaaggctgggattatatgcttgctctttttaaggtggctgtagggacaaactttccacctactccttgtcaagccagtggaccggtggtcccagacatacggctaaagtcaagaggtgatgtcttttggagagatactttcaatcaggaatttcaatcagaaattcaatcatgtggagagagacttatcctaaaaatgtggtggtgcgtgggatgctctgttttattagttccttgacagtatgtatgtgtgtgagtgtgtgtgtgcgcgcgcgcacactcatttggatgggtgtgtatgtgtgggggggggtggtgcgtacgtatgtggatgtgtggatgtggtgtgtgggtgtgcgcgtgcataggtggaggtgtgtgtatgggtgcgggtatgtgtgtgtgttgggcatggagatattgacagctctcccagggctgagtgaaggctttcgggcaaagctcctgggagctaggcaaagctgagttgattcctggttatgccatttattattgggttgcaccgtgtgaaactgccaatattctacactttgacttttatttatttttatttttattttttttgagacagagtttcacacttgtcacctaggctggagtgcagtggcgcgatctcagctcactgcaacctctgcctcatggattcaagtgattctcctgcctcagcctcccaagtagctggaattacaggtgcccgctaccacgcctgactaatttttgtatttttggtagagacgggatttcaccatgttgtccaggctggtctgaaactcctgacatcaggtaatccacccacctcagcctcccaaagtgctgggattacaggcatgagccactgcgcccggcccattttgacttttaaaaatgggagtttgatataattcaatccagtggttgaattagctagcatcgttccctctccaagtctcaggttctcctacacgttagagtcaaaagcagggctatgggaagattaagtaaaataaattttgaaaatgccttatgaaaattacactccaaagaactcgcgccagtgtcagtgttctcatgttcctcatctcacatgatcacatttcgcggattaggaagctgagtctgagaagctccgtgtagtgctttttcggaggcaccgtgatgtgatggaaggctcactcgttaggaagtcagaacagagtctctgagggatcatctccttaatctgtcagtttcctcatctctgaagttgggctcatttccttccttcatggagttattgtaaagatgaagataaataacgtgtaaaatctagcatgggaactggcttctataaggttctaataagtgcattcctactccttcccctcagccttcccatttgtaaaagcaaggcaggggtgaggtgatttctggggctccttttggctctgacatttgaggattttgtatacttttttttttttgagacagagtcttgctctgtcacccaggttggagtgcagctcaatgcaaattccgcctcccaggctcaagcaattcttatgtctcagcctcctgagtacctgggattacaggcaggcaccaccacccccagctaatttttttgtattttcagtagagacggggttttgccatattggccaggctggtcttgaactcctgacttcatgtgacccacccatctcagcctcccaaagtgctgagatgacaggtgtgagctaccgtgcctggccaattttgtgtgctttaatgcccttttctgctggaagagttggcaccaggtttggtgatctctttcccccacacggctctgcctcctgccagtcccagaggggaccctgtccttgcatttcacaggattctgctgttgcaactgaaattccagtaggtcaaagtgaaatttctcatacactttaacatgaagataaatgatcacagtatggccctttaggatcctgagaacatcacggtcatcccctggtataattttaaaagcagatgaatccatgcctgtgcgaggtttgccaggaaagccagtgctgggattacagtggaagtctttttatgctacttttttcttgtatccctcaccccatggggtggcatattgaaaggcaggatgtgtgaccacgatacttttctcctcctggactatgtctaagagtctgttattgggttctgaagatcagagtttaatttccgactcctctctgtgtagctctgggatcttggaaagccacttaacctttctgaagtcccctttcctcatctctaaaatgcatacactcatcactaacatttactgagcactgacatgtgccagacaccattctaagcattttacacagactacaccatttgatcttccaacaaacagaacactgaaacgcattacaggtcagaacaaatgatttgtgcctaagcaccaagaccgtagagcccgtgctccctattctaccctatcctgtctctcaaaatgattgtgagaatcgaatgagacactaggtgagaaaagggttttataaatagcattttaaaaattttttaaagtccacaaaatttttaattttaatacagataaaatagatccctttgttttataaaaagtaacaaaatttgttatacaacaactatgttatttattaattttgcctttttgtatgctgccaggaaagaaacattaagaaatcttaaattgattatggtgaatcagaaggtctgcctggactttttattgctctaactgtacagctgatcatactacctcatttttttttatgacacttcaagggtgcgcttagcttcatcactccttcgttgccaaaagctttgtgaccaaaaacaattaagcagattcctgagtcactaaatgacacataaccagagttgagacttaggaacttttagtgccatgctaagcccacagggacacaacaaatagcattttacaaaggcaaagaattgtgacacttgagatttagcttgttgatccttgtaaaagttttctttttaggcataattgagttttagatcatagtactcactattacttagtaataatttttttctgatagaaatacagtgtaacaggccgggcgcagtggctcatgcctgtaatcccagcactttgggaggccgaggcgggcggatcacttgaggtcaggagtttgagaccagcccggccaacatggtgaaatcccatctctactaaaaatacaaaaaattagccaggtgtggtcgtggattcctgtgatcccagctacttgggagggtgaggcaggagcatcagttgaacccaggaggcggaggttgcagtgagccaagatggtgccattgcactccagcctgggccacaaagcgagactccacttcagaaacaaaaaaaaaagagagagagaaaagaaggaaggaaggaaggaaggaaggaaagaaggaaggaaggaaggaaagaaggaaggaaggaaggaaggaaagaaggaaggaaggaaagaaggaaggaaggaaagaaggaaggaaggaaggaagggtaacaagcaaagtgtaacaatggcaatatctaaaaaaataggtatttttatatgtttgtcgttttatatatatgacccccactttagagatgaggaaactgagagattaaggaaacgatccctgagagactctgttctgacttccaaatcggtgagctttccatcgcatcacggtgcctccgaaagcatgacacggagcttctcagactcagcttcctaatccgctaaacgggattatgtgagatgaggaacatgagaacgctgacatgggtgagggttccttggagtatcattttcatgtggcattttcaaaacttattttacctaatcttcccaaagccctgcttttgactctaatgtgtctcctgagacttggagagcgcaagatgctagcgacagagcaagactccatctccagataaataaataagtaaaataaaaaagaacacaaataattttgaaaatttttttgaaaattaggcacgtttgcactgaccttcaattgttattaattgctggtttcccacccagaattaagttggaatgcaactttcttttacaatcagagtccgttcttggtcttggaaacttctgaggctcctgtgctaatcacactcttgtatttttggcacctctaccccgtgccactgtcatggaacccaggctgatcgcacctattagtggagaaatctgtccataatactgaagtttggggacaaacagtgttcccttagggtaggagaaagagatctttatttttgacaaagggggaggagccagaaaactccagagacccctgagtttgccctctctccaaggtttggggtaagccccccgtcaccctttatctctggggctttcacatattctggattctctcctcctgtttcccagcagaaaaggatggagcctcacagattcttcccatttctggagaaaaacatgcatggagctcaaagttcttctcaggagttttattgccaaagccataataagaaagggtggaggtgacaagcagtgaggaagtttaaagatgcatgaaatctgtaaagtctcagaacaagaattctcctaaaatgcaaaaggggctttgctggtctccccttggcttctcatgtagctcacctcttttttcttatcttgagactagtcaaacctaagctgtttctcattttatttccagaagctattgagaacactctcctgaattcttcaaattcagtagagggcgacaaatgtacatataaatgatggtagtgggtcttaaataaagactcatgacacctaaaggggcagcacctgagtctgattgcacctgtttctgttgctgtttctgtctctcttctctctgtctgccatttcattatcaatggttactttacttataagatcatattagaacctgatatttgataaatgatgcatcagatctatagtgagagaaaaaattaatgcaattaaaggtgttgtaacagctagtcttcaagtggggagaaatcatttgagtaccttaggtcacagcttacatcaaaacaaaaaatcagagctacattaaaaagtgaaattttaactatatcaaacaatagaaaaaaacagaagaaaattgaatacttactaaatcttagcatgaataagaactgtttaacacttagaggcaaggactgggcgtggtggctcatgcttttaatcccaggactttgggagcccaaggcgggcggatcacctgaggtcaggagtttgagactagcctggccaacatggtgaaaccccgtctctactaaaaaatgcaaaaattagctgcgtgtggtggtgcatgcctgtaatctcagctacttgggaggctaaggcatgagaatcgcttgaacctgggaggtggaggctgtagtgagccgagattgtgccactgcactacagcctgggtgacagtgtgaaatcctctctctcaaaaaaaaaaaaaaaaaaaagcaaactagagcagtgaggtaccattatttcctttgctcactaaactgacaacacacaaatgttttttataatacccaaagctgatgagggtagttaaggtatgcccttttatacacacactaatgatgtactactggttggcagtataacatatgctgccatgtggggatatgtatcaggagacttaaaaatgtgcataccttttggtccagtaatttacttctgggaatctgtcataacagaataataatcttggggaaagctacatgcctaaggatatttaaaatattatttaaaaatcaaagtataatttcttacagaatataaaataatattttaaaatgaaaatatgctaaaagtttgatgaaatataaatggtcaaatatatattgattatatccacttactagactagcactcactctgagacgttaaaaatagtcattataaaaactagaaaatgccaaagacaaaataaaggaataaagttttacataaagtatgattccactatgtttaaaaataaacagagacattcttggagttgagtattgttttcttttctgtcatgtccaaagaactatataactattatttttaatgaactatatatgtaatatatacatatagtttatatgtatatacaaaatttatctcatatatatgataaagatgaaagatgagttggatgtgccacgtgaagagggtagtatagaaccccaggtaatggggcataggagtgggattccagataccaggcccatgtttttggggtgagattgccaatcacggtctttcttccatccctcacagaggagtaggtttgtcttcaacaaaccttcagttgtcctgaagacaaacctaattctggagacttcatataatctagaagagacaagcaaactgatgaaaaatagtgaatttttaaggtaaaataaagtacatggactacactttgtttagaatcagattcttgggattaaccacattaacccacagagggtcttagtgatgcctctaatccaggatcctaggacctatttctctctgtgagatgctttctcccaactccttggtgagagtgggaagactaagacctcagcaatctgaggtggaggcctaagatccccctaagatcggaggcagaatctgagaggggataaaagtccctatacctgtattgggcccttttctgggagggggatatcaaagaatgattttgagacagggaggcttttgactacctgtgccacttgagctctttgctagggctccagaatacatatttcaaatacattccccctccctccttccttccctcttccactcttcctttttatcttcctttcttcttttccttcctccttcccttcctttctctggctctctcatgatttcttttcctcattataaaagtgcttatttagtccctactctgctattagtgtgttagtctttgtcccctggtacttgctgtttaatggagaaatgggtgagcaaaacagaaattacagcagagtgcaatgatagagctaagccaggtgtataaatccattctcacactgctgtaaaaaactacttgagactgggtaatttataaagaaaagaggtttaattgactcacagttccacaggctgtacaggaagcatggctggggaggcctcagaaaacttacaatcatggtggaagaaagagcgaaggggaagcaagcacatcacacagcagcaggagagagagagagaaagagagagagagagaatataggggaagtgctacacactttcaaccagatcttgtgagaattcacctactatcatgagaacagcaagggataagtctgcctccatgattcagtcacctcctaccaggccccttctccaacacatgtcgacgtgctatttgggtggggacacagacccaaaccatattaccagggcactggagaaacacagaggggaaagaaccagccaaggagtgagatggagaacaaggaggacttcttgaaacagatgacatccaaactgggtcctgaaagctgaatagagattagacaggggaggaggggcagctaaagatggctcaggcaaacaaagggccaggggatatgttcatgggatgatgtgtctctcgttgtctgcttaacacaaggtgagtctctccctccctctctctctctttttctctgtgtgtgtttgtgtgtgtgcatgtgtgcaaatgtaatatacccaatagtcaaacatgtgccccaggagaggggtagaggaagaaagagaatgagagagtaagaaggaggaatagacacagaaaatgagagagaaggggggaaagaaaaagaagaaaggagccagaggagagaagctggttagcattgaatggagcaatctgtgtcatcgtacttgggaaacccaaggatggattcttggcaagtcgactcttggagctttccctgtgcttggtcctgtgctcagacatgggaaaattagaggagtgtcatctgtgcaatcactgaattcataatcttggtgaggaaaggagactacacacagggaataatgctaagtattacagatttcagggcagaaagagatcaaggtgggctgcaatattcagaaaagtcttcctggaaaagttgaatacttagaaagcagctcctagaagtagactctgctgagatggacggagtcctttgtaggtcccaactgggtgtgtgtgtggggtctgtctctccatggctgacagtgcacatgtggattccagggctcaggatgctgttgctgggagctgttctacwgctattagctctgcccggkcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtaagaatgtttctggcctctttcatcacagacctcctacactgatataaactatatgaaggcattcattattaactaaggcctagacacagggagaaagcaaagcttttttatgttaaccataagcaacctgaagtgatttggggttggtcttccaaggatgagtgtagatggtgcctctataaccaagactttggctttgctgcatctgcagctccttttccatcccctttcccatcttcaccctcatccctattcccagtacattcatattctgattcctctttctgtctgcttaacttccatttcacccagtggcattcaaccacatttactgcacaccccctgaaaggctcagtcctgcctttggggaactcttgatctaggtaagatgtctaatgtgcaaggctctgttggtggttactacaagaaagtctactctaaaaatgtcaaactgaatgtgaacaagtattcaaagtatggagcatagagaaaatatactcaccgtggacctgatgaagaatgaaggcttcaaggaggaggcagagcttcagctaggccttgaatgatgggtaggcagaatagaggaggagagacatcctagatggagggggtagaattgcaaaaccagggttgatggtgccagcacataaagggctggcagggtggagggtctatgatagagacctataggagataaagatagagttgaaattatgggagcctccatgtctgtgggagatatagaaggaggaggtaacacctctctccttttgggagctcttattggtttcttgatctataagtcaagaaggttgtgagtgggagccacagggatggtaatttaggctgtaaccaacctaggcaggagttctgttctttgtagtcactgaggtcttctcattccttaggtcttattggtcctaagggagacatcggtgaaaccggagtacccggggctgaargtccccgaggctttccgggaatccaaggcaggaaaggagaacctggagaaggtgcctatgtayaccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctrtatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactgatcaccactaactcagagcctcctccaggccaaacagccccaaagtcaattaaaggctttcagtacggttaggaagttgattattatttagttggaggcctttagatattattcattcatttactcattcatttattcattcattcatcgagtaactttaaaaaaatcatatgctatgttcccagtcctggggagcttcacaaacatgaccagataactgactagaaagaagtagttgacagtgctattttgtgcccactgtctctcctgatgctcatatcaatcctataaggcacagggaacaagcattctcctgtttttacagattgtatcctgaggctgagagagttaagtgaatgtctaaggtcacacagtattaagtgacagtgctagaaatcaaacccagagctgtggactttgttcactagactgtgcccttttatagaggtacatgttctctttggagtgttggtaggtgtctgtttcccacctcacctgagagccattgaatttgccttcctcatgaattaaaacctcccccaagcagagcttcctcagagaaagtggttctatgatgacgtcctgtcttggaaggactactactcaatggcccctgcactactctacttcctcttacctatgtcccttctcatgcctttccctccaacggggaaagccaactccatctctaagtgccgaactcatccctgttcctcaaggccacctggccaggagcttctctgatgtgatatccactttttttttttttgagatggagtctcactctgtcacccaggctggagtacagtgacacgacctcggctcactgcagcctccttctcctgggtccaagcaattattgtgcctcagcctcccgagtagctgagacttcaggtgcattccaccacacatggctaatttttgtatttttagtagaaatggggtttcgtcatgttggccaggctggtctcgaactcctggcctaggtgatccacccgcctcgacctcccaaagtgctgggattacaggcatgagccaccatgcccagtcgatatctcactttttattttgccatggatgagagtcctgggtgtgaggaacacctcccaccaggctagaggcaactgcccaggaaggactgtgcttccgtcacctctaaatcccttgcagatccttgataaatgcctcatgaagaccaatctcttgaatcccatatctacccagaattaactccattccagtctctgcatgtaatcagttttatccacagaaacattttcattttaggaaatccctggttttaagtatcaatccttgttcagctggacaatatgaatcttttccactgaagttagggatgactgtgattttcagaacacgtccagaatttttcatcaagaaggtagcttgagcctgaaatgcaaaacccatggaggaattctgaagccattgtctccttgagtaccaacagggtcagggaagactgggcctcctgaatttattattgttctttaagaattacaggttgaggtagttgatggtggtaaacattctctcaggagacaataactccagtgatgttcttcaaagattttagcaaaaacagagtaaatagcattctctatcaatatataaatttaaaaaactatctttttgcttacagttttaaattctgaacaattctctcttatatgtgtattgctaatcattaaggtattattttttccacatataaagctttgtctttttgttgttgttgttgtttttaagatggagtttccctctgttgccaggctagagtgcagtggcatgatctcggcttactgcaacctttgcctcccaggttcaagcgattcttctgcctcagcctcccgagtagctgggaccacaggtgcctaccaccatgccaggctaatttttgtatttttagtaaagacagggtttcaccatattggccaggctggtctcgaactcctgaccttgtgatctgcccgcctccatttttgttgttattttttgagaaagatagatatgaggtttagagagggatgaagaggtgagagtaagccttgtgttagtcagaactctgtgttgtgaatgtcattcacaacagaaaacccaaaatattatgcaaactactgtaagcaagaaaaataaaggaaaaatggaaacatttattcctttgcataatagaaattaccagagttgttctgtctttagataaggtttgaaccaaagctcaaaacaatcaagacccttttctgtatgtccttctgttctgccttccgcagtgtaggctttaccctcaggtgctacacagtatagttctagggtttccctcccgatatcaaaaagactgtggcctgcccagctctcgtatccccaagccacaccatctggctaaatggacatcatgttttctggtgatgcccaaagaggagagaggaagctctctttcccagatgccccagcaagtgtaaccttgcatctcattgctctggctgagttgtgtgcctgtttctgaccaatcactgagtcaggaggatgaaatattcatattgacttaattgcagcttaagttaggggtatgtagaggtattttccctaaagcaaaattgggacactgttatcagaaataggagagtggatgatagatgcaaaataatacctgtccacaacaaactcttaatgctgtgtttgagctttcatgagtttcccagagagacatagctggaaaattcctattgattttctctaaaatttcaacaagtagctaaagtctggctatgctcacagtctcacatctggttggggtgggctccttacagaacacgctttcacagttaccctaaactctctggggcagggttattcctttgtggaaccagaggcacagagagagtcaactgaggccaaaagaggcctgagagaaactgaggtcaagatttcaggattaatggtcctgtgatgctttgaagtacaattgtggatttgtccaattctctttagttctgtcagcttttgcttcatatattttagcgctctattattagatatatacatgtttagtattatgtcttattggtgcatttactctcttatcattatgtaatgtccttctttatctgtgataattttctgtgttctgaagtctactttgtctaaaaataacatacgcactcaacttccttttctttcttccttcctttctttcttccttcctttctttctctctctctctctttccttccttccttcctccttttctttctctctctctctctctctctttttttgacagactctcgttctgtggccctggctggagttcagtggtgtgatcttggctcactgctacctctaccatgagcaattctcctgcctcagcctcccaagtagctggaactacaggctcatgccactgcgcccagctaatttttgtatttttcgtagagacggggtttcaccacattcgtcaggttggtttcaaactcctgactttgtgatccacccgcctcggcctcccaaagtgctgggattacaggcatgagccatcacacctggtcaactttcttttgattagtgtttttgtggtatatctttttccatcatgttactttaaatatatctatattattgtatttaaaatgtgtttcttacagactgcatgtagttgggtataatttttatccagtctaaaaatatctgtcttttaattggtgtttagacaatttatatttaataaaattgttgaatttaagatggatgactgttttatttgtttgctgttcaccacttctgttttattctctttccagaattcttttggattgtttaaatatttcataatattttatctt

SEQ ID NO: 2 - protein, 244 aa, Homo sapiens
  VARIANT (9)..(9): Xaa = Leu or Gln
MLLLGAVLXLLALPGHDQETTTQGPGVLLPLPKGACTGWMAGIPGHPGHNGAPGRDGRDGTPGEKGEKGDPGLIGPKGDIGETGVPGAEXPRGFPGIQGRKGEPGEGAYVXRSAFSVGLETYVTIPNMPIRFTKIFYNQQNHYDGSTGKFHCNIPGLYYFAYHITVXMKDVKVSLFKKDKAMLFTYDQYQENNVDQASGSVLLHLEVGDQVWLQVYGEGERNGLYADNDNDSTFTGFLLYHDTN

SEQ ID NO: 3 - protein, 386 aa, Homo sapiens
MNEPTENRLGCSRTPEPDIRLRKGHQLDGTRRGDNDSHQGDLEPILEASVLSSHHKKSSEEHEYSDEAPQEDEGFMGMSPLLQAHHAMEKMEEFVCKVWEGRWRVIPHDVLPDWLKDNDFLLHGHRPPMPSFRACFKSIFRIHTETGNIWTHLLGCVFFLCLGIFYMFRPNISFVAPLQEKVVFGLFFLGAILCLSFSWLFHTVYCHSEGVSRLFSKLDYSGIALLIMGSFVPWLYYSFYCNPQPCFIYLIVICVLGIAAIIVSQWDMFATPQYRGVRAGVFLGLGLSGIIPTLHYVISEGFLKAATIGQIGWLMLMASLYITGAALYAARIPERFFPGKCDIWFHSHQLFHIFVVAGAFVHFHGVSNLQEFRFMIGGGCSEEDAL

SEQ ID NO: 4 - DNA, 21 bp, Artificial
  primer
tttgacttttgccccatcttc

SEQ ID NO: 5 - DNA, 20 bp, artificial
  primer
tgcatatctgcctccaccag

SEQ ID NO: 6 - DNA, 21 bp, artificial
  primer
gggcagaaagagatcaaggtg

SEQ ID NO: 7 - DNA, 20 bp, artificial
  primer
tggaagaccaaccccaaatc

SEQ ID NO: 8 - DNA, 26 bp, Artificial
  primer
ctataagtcaagaaggttgtgagtgg

SEQ ID NO: 9 - DNA, 23 bp, Artificial
  primer
atctggtcatgtttgtgaagctc

SEQ ID NO: 10 - DNA, 97799 bp, Homo sapiens
gtgcgatgcgcgtcacggcgacgtgcggtcgtgggggcggggagacgcggcggcggcggtcgctgtcgcggcggcgagagcgcgattccagaagcggcatcgcggcggcggcagcggcggcggctacaccgggcttggccccctccctcctccgttcccccctcctcccccctcccctcagcggtggctcccaagaagtccgagacacgcggtgaggcggcgacgggctccgggtgagggtctctggagtgtgggcgccgtttcctgggcgcagagggagagcttggggtcggggggaggtgagagctgaggggcgaatgtccctggcgcgggcgggagagcgcgctctcggcccggcccgcaacttgattcctaactctgacctgccgcccggcgcgccttggcctgagggtcggccgtgtggggacctggagccgccctccttgcgaccgctgtcggaggagtccggcgcccgctgttcccctcgcctcgcctttcgcggcggagggtcggagcctgctgccctctgctcaccttcgggtgcagttgctccagctccctgcccgcggagaggatctgcccgcccctcttcgtctcttgtctgtttttatcacttctaggaatttgtttggtggaggcttgggatgggctctctcagcgctactttcacagtgactggggtggggaagagttccctcacctttttcaggtggcgcgtcaggcagcctgcttggtacctggagtcctgttttcccaagcccccgtccgtcagtgttcgcttcttctaaaattaccgatgacagggttataaatcagtttttagggggctcggagagagtttgggttggaaactgttagctcttttttcctcctggcttttgatcttgatttaagcgtttgcctctgaaattttgacaacttcataagatggtccaaaagtgaattttggtagaaagatttaaatgcacagttgggattttatgggggtacggtgacttcattgcaggttgttgactttttttttaaaataagatgttcttttaattttttgactctgggtttctgagccttggttattgtcccccatttctgttccaaaagcagactttttttttttttatcaccccttcccctcacccagagaatccctttaaacgccaagaaagctgtcataacatttagcattatttgatcacagaacttttccaaatgaaatgctaaaaattccttcagaaaaaagcatccatctacccatagcattattttttggggggaggctatacgtcttgtcagttctgaaagtttgaagtgacctcttatcaaaggccacccttatgtctccttaaacgactcttaggtaattcctagatgttttcatccgttctgataaatttgttgtcagagtgttgattgtgatgatctgcaactaaaattatttcatcagtaaagatttccacacatgtggcagctgcttgttggtgaattatgaaatttgaaaaaattgcaaattaaatgtgaaaggcatatttcttttaacttctgattgtaactgtctgtcctccagtatacgttcctatctagactttggtctgcttagcagatttcctcttttcacttgacactaggaatgggagtaggatttccagagcctaaaagagagtttgttttttttttctatccctaggctccttgtatgaatttataaataaatggttttttctatcaatagtatcaatttagctggtgcactttttttttttagtataaatcacattttgtttcctaaaatgaaaataattttgttttattctttatttcagggattgacagattttaaagcctgcaggcccagtcctgccagttgcctctttttgtaaataaagatttgattaggcctggtgcgatcgctcacgctggtaatcccagcactttgggaggtcgaggcgggcagataacctgagatcaggagtttgagaccagcttggccaacatggtgaaaccccgtccctattaaaactacaaaaattagccgggtgtggtggcgcactcttgttatcccagctgcccgggaggctgaggcaggagaatcgtttaaacccgggaggtagaggttgcagtgagccaagattgcaccattgcactccagtctggacaacaagagcgaaactccgtcttaaaaaaaagaggaagaaaaaagtttttattagaacatagccatatgcagtcgtttaggtattgtctgtagtttcttttggggtacagcagcagagttgagtagttgtgccagagagccatgtggcctgtaacaccttatatttatttactatgtggaccttaatataaaaagtttgccgagctccattaaaaatgatacatagtcaatgttgaaaatttaaatgagtcagaaaattgtaaaggagaaagtaaaagttatttacaagtacaccacttggggctttttttctgtgattatagaatttgttggtgattattttttatataaatgaactcaaattgataaatgttggtttgtaatctgcttttttttttttttgttcattttgttttttgagacggagtttcgttcttgttgcccaggctggagtgcaatggcacgatcttggctcactgcaacctctgcctcctgggttcaagcaattctcctgcctcagcctccctagtagctgggattacaggtgtgcgccaccaagcccggccaattttttttttgtatttttagtagagacggggtttcaccatgttggccaggctggtctcgaactcctcatgtgatccacctgcctcacccctcaaagtgctgggattacaggtgtgagcaaccgtgcccagccctgtaatctgcttttttaagaagtatttcattatttgtatttggattttgattgcttttgtattttctgcattggaggtctttctagaaaggggcttatgaaccgggttttcatgattagtcttaccagtcaagatttaaaaagaaaatccaggaataataaatgcttggtaatgaagatgctggttaacatttattgagcacttactatgtactagacaatttccactactttacgtgtatgaaattatttaattgctgtggcagctctgtgagtgtatactatgatttctgttttacaggtgaggaaactaaggcaaggagaaaaattaagtaactgttggaagtttatcagctagcaaatggtggagctaggatttgatttcatgcagtctgatttgagaaacccaaattcttaacaatatgctacagagcctctacttacattattgctcagtgtagaaagtttagacagtccagagatatactgtcatcccttggtgtctgtagggactgcttctaggacctttggtgaatatcaagatgcacagatgctcaaatctctgatataaatggcatagtatttgcatataacctgtgcatatcctcccaaatactttatatcatttctagattacttataatacctaatagaatataaatgctatgtaaatagttgttatactgtattgtttggggaataaagacaagaaaaaaaagtctgcatgtttcaaatacagatgctatattaaactttttggatccaagcttggttgaatccacagacataagagcccatggatatggagggccaactgtataatgtaaaaaaattccttctcatgcgctagtttccagttttcatcctttgtagataaccaggggcaacagtttgatataggtcttttcacctctctttgtgtgcatatacaagtaattatttgcagatttgtctgcatattcagaatttaaaaattacacttttttttagcagctttagatgtacagaaatttgagccgatagcacagaaagttcccatattcccttcccttccctcgtttctcctattgcaaacatcttgcattaatttggcacatttattgcatttgatgagcccatatcaatatgttgttatttattggtctatagtttacatagggcttattctttgtgttgcagttttttttttttttttgttttgtttttttaagatctcactctgtatctcaggctggagtgcactggtgccgtcatggctctgcagcctcgacctcccgggctcaagcgatcctcccaccttagcctcccaaagtgttagggttacagacatgagccactgcacttggccgagagttattttttacaaaaatgggatcaagcccagtggtggcttgcgcctgtagttccagctacttgggaggttgaggtgggaggattgcttgaggctaggagtttgagaccagtctgggcaacacagcatgactctgaccagtctgggcaacacagcatgactctgtctcttaaaaaaaaaaaaaagtgggctgagggtggtggctaacgtcggtaatccttgcactttgggaggccaaggcaggtggatcacctgaggtcaggagttcgggaccagcctggccaacatgaatgaaaccccctctctaccaaaaatatattaaaaaaaaaattatctgggtatggtggcgcgcgcctgtaatcccaactactcaggaggctgaggcaggagaatcgcttgaaactgggaggtggaggttgcagtgaaccgagattgcaccactgcgctctagcctgggcaacaggagcaaaactccatctcaaaaaaaaaaaaaaaaaatgacgaaacccgtcattcagcagtcattcaccttttttggtggttttcaggatatagtgcttttggactttctaatgtaaacattcttgatttactaattttcagattaatgacaatttattttgggaatggaaggctggcttggatgttactttttctctctttttatttttctatttttttttttaagtgtatctcatagtggcagcagcttttccccaaaggaatgcttattttttttgacgggacattctggctatacctaatcatcaaagttcttcttttttctctttatctctggatttggaaaatgattcattttggttttcttcctcataaattctttttggtctgtccagccattgcagttgctagtcatgtctccttgtcagactttggctttttatgactgtcatcaatgttagaaaaaattacttgctccttatcactatttcaatacatttcctcttgggctttccctgggggcagcactgaattttttccttagttgagccatgcagattttctagaagtcatcgtttagtttggcttttcactcctccatgggtgctgttcagcttctaggagatttcttgcagtttctacttgtcaacagattcaccagcagcaccctcctcctcactgagactggtgcttccccacccatctttatcattttcagccttctctttttctttctcttttcttttttccttcttttttttttttttggttacagcgtcttactctgttgcccagactggagtgcagtggtgcaatcgtagcttactgcaacctccgcttcatgggctcaagtgatcctcccacttcagcttcccaagtagccgggattacaggcatgcaccaccatgcctggctaatgtttgtattttttgtagaaacagggttttgccatgttgtccaggctggtcttgaactcctggcctcgagcgatcctcctccctcagcctcttgaatagctggatttaacaggcaggagccattgtccctggccatattcttgcactgttgattttactggggcctctgtcagcttaccctggaattctgcatcatctgaggatttggaagaactgaatcacagttatagtagacagcatcatattcttatccttgtgtgttttatactgggacaggtcttgaggaagttcttctgtcatggctaggggacattgagctattacctcttatagcactgatttggactgtcatgtcttccctaaagttcttgtccataacataatggatggtcacagtcatgagcaataactgagtgatctctgggactaacaggtgatgaaatgcatgtttagcaaacagtgatttctgttacttctctgtggtgcagcagcagaaacttttgcccaaaggcatagaagttagagggcttatgaatttctgccaatctggagataagatttatgagcatcatgttaactcaaacctctgcttagagatttctagctgtgtgcttcaggagtgttttggtaaaactttgaggatcatgaataaagctaatagcttaaaagttaaacactgtgagttttttcttcttttgtttcctgggcacttttcataaccttttcagtgttttgtgtttggagccactcttcccagtgacatactgcacttgtaggtctcttgtcactttatcctcagattctgagtctgtcctatttgtttctcttcttcatcttatcctaggaagaatgtcaaggtggcaaccaatatctcaaaacatgcagttgtgatgacattgacagttttggcatcagtccggatgtttcttgtagagttcagtcatcacatccaaagatatcttggctgcagttacattgctgtctcttaacacgatgtatatgaaattctgcagtactatatccactttattcttgtgttttacagttatattcttgctatcagtcacaacatgagtatgtaaagtctcaaaagcttattatggcaacttggaagttcaaaaaagagctctagcaaacttgatggatggatatgatacttctcagcaagatcaaagctttgtaaaatgttattagatgattttgattcaagacagtacagtataattgtaggaaagtagatcttttagctcttgaggaaagttacttagatgttctgggtaacagtgaccaatctgtgccataaatatcaccagctctgtcaattctttgccaggtttattggttttaatttgagaatctccatattggatttgtagtgtatatagtactgctgtagtaaccctttaatgtcagccagtggctccactttattagattctataactgctgcttgttactgggcagctagtttctgcaggacatgttggctgtagactggatccacagtgagtagttctcagagaacccgtactgctgacatttgcaaacccatttcagctgtggtgtggagctgtggcgttgtacttctggatgtgaccagaaacagtgtttgggagctcagcaggtggtgctgtcttggctgattgtgtgtgtgtgtgtgtgtgtgtgtgtgtgtgtattttatttaatttctgagacagggtcttgctctgtcacccaggctggagtgccatggcacaatcacggctcactgccctctcgaactcaaacagtcctcccaactgagactcccacgtagctgggactatagatgcacgccaggctaatttttgcatttttaatggagacgaggttttgccatgttgcccaggccggccttgaactcctggactcaagtgatcctctttgccttagcttctcaaagtgctgggattataggcttgagccaccatgacaggccctgtgtgtgtgtttttaaaattaatatttgtggcttactttacatctgcatctactctcttctttttagtagttctctagtgtagtattattaaatggatatgctataacttaatactgctcttactgatggatatttagattatataaatggattgtaaattgcccatacattttatgtatatttattgcaagctaattcccctttagtaattttgtaatagttgaagaaaacacatttttcattttgatgtgtgttatcacattgccacccagaaaggtcacagcaatgtaaactctcaccagaaatgtgggtggagtctagcttctctgtgtcattacaaattaaaaaaaaaaatctttgtttatctgataggaggttaagcatttgcagtatattctatctaactgggagatatgctttgtttcaatagcattcttttaaagttgatttacgattacttcaggtggagcatgcattctgttcccgggcctcccttaggtggagctgcttcacctatttacattctctgcttggctcttgaaagcatatggttttgttatcactgatcaaaaagaatctgggtgcctgcctaatagactatatatttcttgagggtaaggaacgtgtaactttcttgataatctactggcagttccggtatatagcaggtggtaagtaaatggtcttttaaaaatctaaattggttcatgaaccagtaactgttggggttactgctgagtgaagtgctttaattttgttaaaaataatattttcaaattgtgaaatacaaacttgaaaaactgcagtgcatgctgggtgcggtcgctcaggcctgtaatcccagcactttgggacgccaaggtgggcggatcacctgaggttgggagtttgagaccagcctggccaacatggcgaaacccccgtctctactgaaaatactaaaacaaaacaaaacaaaaaaaaccaaaaacaaaaattagccaggcatcatggccactgcctgtaatccctgctacttgggagaccgaggcaggataatcacttgaacccgggaggcagaggttgcggtgagccgagatagcaccattgcactccagcctgggcaacaagagcgaaactccgtcttaaaaagaaaagaaaaacttcattataaaattgtcactttggcatatgttgtctctcatctttcaactctaagataagttaaataggacgaactgatatcttaggacctgttaggaaataattagcctattttataagtacatttggtatttagaaattggaagttgaggaatttattaaaaaggggatttgtttaacattacttatggttttgagttttctggattggagtataccatgtgttagaaggggatatatgttggccaatgatattatagattggcttaattagacaagtgtccagacagaaggaaaagggaagctaaattctgagaattgccagattatcttcaggcatgggaaaaataactggaaaaggacacttaagttttttaatggaagactacaacaaaatgatattttctttataactgatcatattacacattcttactggatttattgaatataagaatattcttatttccacaatgtctgtgtactagagaacaaaacacaaagcatatggtcttaaaaggggcaaaattagttagcttattacactttggccatttaggcctttcttttgtatgtttgttgttagtatatcagctgattatttttcattgacagcatattcactttaatcttgtatgttttgaaattaagtttagtgaacatgtatgcctagttgtatccatgtatccattcctttggttaaggaggatgtctttgtggtaggggttacattatgaaaacttcagcagaatggcacagaagattgactttattttcttgaaatttattttatcctatttcttccagaaaggatatctggtaacttaaaataaaagatctggaagtgtaataaaatatgtgtaaaaagaagaaatcagggaaaggacaaagcatatattcttttcccaagtgacagcataattcaatgactaagaataaaaattgggtttgagtttgcttggcagcgagggtaaaaatggaagtatggtaaaatacatagcactaaactaaaaggaggaacaagaccaatttttcaggggtgaaatcctctcttcacagatttctaagaaactatttaaactggacattatataaaagctgttttgttgttgttagtggccctagatttaaaaaaaaaaaaaaggtgagggctaagcattaagatttcaagccaatgaaatacattttaggggttgatttgaatttaggctgcagatgtatgttttatttggccaatatatgttttttttccttaaaatatttcaaacatacatgagactacagagaccaatataattaacattcatatattaaccatacagctgtagcatatcttagcatgttcccatatttctcatttttttgaggaaaaaaaatcatgtacagttgaagcaacttcttggattacagtttactttcttcccagaagttaactattattctgagtatggtgttgatctttttttttttttttttttggaaacagggtctcactctgtcacccaggctagagtacaggggtatgatcatggctcactgcagcccatctcctttgcttgagcagtcctcccacttcagccttccaagtagctgggaactcaggtttacaccactgtgcccagctaatatttgtatattttgtagagattgggtttcgcctgttgcccaggctggtctcctgtgctcaagcagtccatccacctcggcctcccaaaagtgctgggattacaggtgtgagccaccactcctggcctgatcatttttaatgcatgtttttatgtaattactacatgggtatatattcacaaatcatagtattgctttgcatattttcagatttgatgtaaatgatatattctgcttattctataatttgtctttcatgaaaatttttttgatttatatgaaactgctgatacgttaccattttgacctacaaaaatgacagtttcatatggcccaccctaatagtcatacttttatctgctgtaaaattattttctattatatgaatatagccagataaaatttatttattctcatgttgataggcatttagattgtttccaggttttcatgtattaaaaataatgttacattgaacattccttattaggaatcattgtgcttacatttaagatttttggcaagttttaaaaactgcatgttgtggctgggcactgtggcttgagcctgtaatatcagcactttgggaggctgaggtgggcggatcacctgaggtcaggagtttgagaccagcctgaccaacatggtgaaactccatctctcctaaaaatacaaacattagctgggcggggtggcgcgtgcctgtaatcccagctactcaggaggctgaggcaggagaatcgcttgaacctgggaggcagaggtttcagtgagccgagatcacgccattgcactccagcttgggtgatgagcgaaactccatcttaaaaaacaaatgaacaaacagaaaaaaccccacatgtcgcgactccctaatggattgtgaaatcaattcaggaattagaaaaatatcaggttgtattgcaaacaataaggataaaatgttttgtgaaatttttatttctgtgtgtatgtatgtggttattaagtttgaaatgaagccctctatggtgaattacccagaagtgcaattattgggctgaagattttggaggttatgggaatttttttagctgtattggatattgtcaaatttgctgtcactttgcatattttagcactagagtatgagaatttgccatcttccgattgttatgattaatggcattgtcagattttttacatgtttacaaatcagatgagtgtgaaatgttaccacattgtcttaatttttatttttgattatggggttgagtatgttttcaaatgttagatgactattcaagtgtttgtttcttgaattggctgttcttttccttccacagccatttgggtggtctttctaaattgattttagaaagttttaagttcattacatacttcgattatgtttgttaagtgaattgcaactgtcttttctcagtctgtgtggcttatctcttaactttatatggtttgttttgttaaatagaaatgattattataaaatatatcactattttcttgtttgtgttttatttaagaaacctggggtgatcactctgtggttctcctcatgcacattaacaagtttgttacttcatttcttcagttgatctcccttttgtgaggtggtttttaagtgaatcattaaagagcaaaggagaagttttcccgtggctcccacgctgctattcaaaacttcctgggttagtaagccttcttgtgaattgtctttaaaatgtttattaaaaactcttaggaactctcatataaagtcttaaatttgtgtacaaagatgctctctgacattgcttgactagtgaagaaagttaaaacaatctaaatgttaaaaatatattatatgtatccagacttcagaatttttttgtagacattaagagaataagctagagctatacgttgtgacatgaaaatgcctactatatactagttaagttttttaaaaagtattttgcagagaaaatatgcacagtataatcttacagtgcaaaatttttgcaggcacatatgtatatgatatatgcaatctatatatgcatatagaagggtctgaaaatatataatagttgccactggagtggtatgtgtgtttgtgttgctttaatttttttcagtacacatttaaacagaagtaaaaaacaatttaaaagaaatttggctgggtgtggtggctcatggctgtaatctcagtactttgggaagctgagataggaagtttgcttgagcccaagggttcgagaccaacctgggcaacagagtgagacctgtctctacaaaaaacttaaaaaattagccaagtgtggtagtgtgtgccgggactgaggtgggaggatagctcgaacctgggaggtcaaggctgcagtgagctgtgattgagccactgcactccagtctgggtgacagagtgaaactctgtctcacgaataaataaataaataaataaataaatgggagaatcttaataaccttggagtatggaatagatttgtttgtttttttgtttgcgacagggctcaagtaatcccaccaccccagcctctgtagtagctaagactagaggcatgtgccaccacacatgattaatttaatttagttttgtgaggacaggatctcactatgttgcccaggctggtctcaaactcctggcctgactgatcctcctgcctcagcctcccaaagtgctgggattacagatgtgaaccgctatgcccccaccagtttttttttttttttttttgggtcttggttctacctggaattttttttttttttttttttttggtgtgtgatgtgaggcagggatcaaatttaattttattatatgtatacccaataaccatattactgttaacagagtgttttgaatttttgagttaataatttttaaaagcagctcatggtttcttgttaagcacaattgcagtctttttgtccttcaccaggctgcttcatatattcaagtttgtgatctctgtgaccagagtgatatgattggtggatgacttgatccagaaatagaacctagaagagtagatacagagtatattttgtaggtctcactcacatgtcatttctcttactatttaaatggaaattagattgcactttctttcttctctaataaacttgctttcacttaaaaaagaaattggattgcagacaatttgaggatatacattaatgaggaaacagattgctggttttttgcattgctggctacagaccttggaaactagagaagcacatggttgattgattgatatcttgttctaaaacttgagcatacttaaatgtatttggtcatccagctattatcacatatactaacgtttgagacttttaattttgactttagggtacatgtatcaatacagttgtcttaactttctaagatgtttgatataaaacactgtaaatcacttagtttaagtatgaagaccatttttgtttcctcttaagagggtttttattacacttttagtctaagaagtgttgctgctgaggccttcagatgtatattctaaatttttcttttttctttttattccttttctttctcatactggtggtcagaagaaggccttatgatttagtagaattgagcaatattgataaagattgtagatacaagtttatttttagatttcttagacttgtaaaattgagaaagataattaaccacttccttgaattttccatttgcagcattataaagcaaaacaaacaaacagacaaaacagacttgtgctctggagggtaatatgtgagatgagatagagccccgaatgattgttatttaataatgggagaaaggtgctttatcacttcaaagtagggttttgattttgcctgaaaggttaactgtacaggccacatgttttatctgaacagattttatttttgaagtagtttaaacagtgggctatgtggtattatagtacagatgatttatggaaggaagtctttacatatttgatatattttggcacaaaatatataatcctagatttttcaggatttccctgactttaatatttagaactgttgttccttgtattagcatttttatgtacagattgagcatctctaatccaaaaatctgaaatctaaaatgctcccaaacctgaaactattcgacgccacaagtggaaaattccatacctgacttccagtgacaagttgcagtcagaactttgtttcatgcacacaattacaaaacattgtataaaattaccttcaggctatgaaacaaatatattttgtgtttagacttgggttatatccccaagatattttattatgtatatacaaatattccagaatctgaaaaaattccaaatccaaaacacttctgggtactaatctttgtttcaaaaacaatagtcattattatagatcttgggaaatgaggataaccgttgtaatatggctgtaaaagagttgtagaaagacttttctcctttaaagacattgtttttgtagctttgaccactaccagtggctataatgcattgtttgtggctttattgaagtcctataagatggtgataaaagcatggactttgaagtcagatagataaagatttgaattcagggtcttccacttaagtttgtgttttgatgaattgcttgatctccgtgaacctcagtttcttcatctataaaatgggaccccacttcataaggattgttggagatgataggtgaagggtcaactactgggccttgttcaaagttgtcttcagtagtaaagtaacaattcaccttctcctacagagagataacatcatgatgtgggaagaagggaactacttacttttgtgggaggcagtgttgtataataggccatttgttgcttctctgttttgtacgttgtgtaaccttagggagattatccagaaagtttaccctcagtcttctcattggtaagtagagataatgttacctatgtaacagcagagttttaaagctaaatgagaatatttataaaagctcactttatctggcacatggtaacatggtaacttctattaataaaattttatggtaataagcaatattatgcttctgaagtaggtcacaagaagggcttttggttggagaaaccagaattgttatgaattagtccgtgtttactttgagggagtaaagactagggagtattcattgaaggaagactgtttaggaggctactattacttggctaaaatacaaataatgtgactcaatagtttctcaaaattttgtgtaatctaggcttatgtaaataaactttactgttttattaggatttaaaatcaactttattgaaatataatttaaacaatgaaatatactcattttaggggtgcagttagatgacttttgataatgtacatacccacccagataaccatcaccctaatcaaggtaatggaatttctgttatctccaaaaccttcctcgtgtccctcacagtcagtgcaccacacccctcccctcctctggttcaaggaaaccactgatttgcatacattcattttgtctcttagaatttcatgtaagtgggctgggcaaggtggctcatgcccatatttccatcactttgagaggctgaggtgggaggatctcttgagccaggagttcaaggctgcagtgggccaggatcacaccactgcagtccagcttagtgacagtgtgagacccggtctcaagaaaaaaactttgtgtgtgtaaatggaatcatactgtacatattctttaatgcctggctgctttcactccgcatgtttttgagagtcctccatgttgctttgtgttccagtagtttgttgccttttattgctgagtagtattcactgtggtatggctataccacaatttgtttcttcattcacctgatatttgggtttccagtttttggctattatgattaaagtggcaatgaacattgatgtacaagtctttttgtggacttaagtttttatttctcttgggcaaataggagtagaatagctgagtcatatggtgtatattttgcagtttcttaataagttaaacagtttttcaaactgattgtgccattttatcttcctgtaatctcatttacattatatgctgtgtttttaattttaacttttttttttttagtgtgaattgatatcttattgtggtttttatttacatttttctttttaaaagttttgttttttgttgtgagacagggtcttgtctgttgtccaggttgatgtgatcctggctcactgcagtcttgacttcccaggctcaaggaatctttccaccccagccttccaagtagctgggactacaggtgcatgccactacatccagctaattttattttgtaaaaatttttatgtagagatgagatcttgctgtgtgcccaggctggtctttaaatctggggctcaagtgatcttcccgcctcagtctcccaaagtgctgggattataggtgtcagccaccatgccttgccttatttgcatttttctgatagcaaatgatgttgaacacttcttcatgtgttcattggccatttgtatatccttttattagatgaggtgtctaattcttaattcatttttaaaaaagcttttaaaaattactgagttacaagttctttatatgttgtggatacaagtactttgtcagctgtgtgaattttgaatatacagtcagcgctccttgtctgagggttccacatccacagatttaacgaatctaagatggaaaatgtttggtaaaaaaataacaatatgcgaataaaaaataatacaaataaaaaatacagtataacagctatttatatagcatttaatcatattaggtattataaataatctagagattgatttaaagtataaggaagatgtgcatatgcaaatatgggttttttttttttttaaataagggacttgagcatcctcgaattttggtgttcagttttgggtggattaggagggtagagaggggttcttgaaaccagtccaccagggataccgagagatgactgtattcttttgttctgtggcttgcctttcattttcttaacagtctttggaagagcagatgtttcttaattttgttgagtccagtttatcactttttacttatggttagtgctttttgtgtcttaagaagtatttagctacaccaaggttgtaaagattttcacctgtgtttattttggaagccttacagttttagacattacatttaagcctatgatctatttcaagttaatttttgtgtaggatgtgaggtaaaagttcaggttctcctctcccatatgaatatccagttgttcaagcaccatttggtaaaaagactatcctttcctcatttaattactttgacctttgttgaaaatcagttgtccataaatgagtgggtctttttattattgcattgatttgtatacctatacttttttttttttttgagacagagtcttgttctgtcatccaggctggagtgcagtggcacagtcttgggtcactgcaacatccgcctcccaggttcaagtgattctcctgcctcagcctcccgagtagctgggattacaggtgtgcaccaccacgcccggctaatttttgtatttttagtagagatggggtttcaccatgttggccaggctggtctcgaactcttgacttcaggtgatccacctgcctatacctcccaaagtgctggattacatgtgtgagccactgtgcctggccctgcatatctatacttatgccagtactacacttgattactgtagctttatggtaaattttggttactctagattctttgcatttccataaattttggtattaatagtgtcagtttctatcaaaaagcctgctggaatttcagtttgtgttgtattaaatatacagatcattagggagagaattgtcgtttaaaaaatattgagtcttctggtccttgaaaatggtgtatcttattattttttaaggttctttagtttctctcagcagtgttttgtagttttcagtgtaggaagtgttggacatcttttattggatttattcctaattattttaagtgcttacatgggaatttttttaaagtttcattttttccaatttggtgctagtatgtagattacaattgattggtggtctgtctagcatgtagaaattccttaataagtagtcagatggaatttaatttgttctcatctaactttctggagaaaataatcatctaaaagaaaaaagattatctctgaatttgaatgggttttaaatttgtatgactgtggcttattttaatttctctgaatttgtaacgtagaagtacatttggattttatggtaaaatatacgatattggatataagtggagacatgtagagatcattcaacagatgagaggatatagctatagaaaagcagagttgaaaatttttagatggtaaaatattcaattttatattaaatataatattttaaagtagaaagtgcttgagtagtatgattaaaaagaggaaacttgttttcatttgaatacattttggcctacaagaatgtctgttactggcttaagtatttttgtttgtgtataccgtcagatgatttaccatttcctagccaaacctgatttgttggcccagtacaactctcttctctcttttaagctgtgtctttgatagtatttctgttcatcctgcctagaagattgtgttctcacacagttttctgttgttggttctaaaacatggtgtaaatgagaggattgtaaaatgcgccaatcagcactctgtaaaaatgcaccagtcagtgctctgtggctagctagaggtttgtaaaatggacccctcagcacactgtaaaatggaccaatcagtgctttgtaaaatggaccaatcagcaggatatgggcggggataaataagggaataaaagctgggcaccccagccatcagctgcaacccagtcaggtccccttccatgctgtggaagcttttttctttcgctcttcacaataaatcttgctgctgctcactctttgggtccgtgccacctttaagagctgtaacactcaccgtgaaggtctgcggcttcattcttgaagttagcgagaccacgagcccactggaaggaaccaactctggacacatcttggtgacccagacgggaccattggaccccttttgcttgctattctgtcctatttttccttacaattcaggggctaaacaccagggcacctgtcggccagttaaaagcaactagcgcggccaccggactaaagacacgggtgttaggctttctgggaaagggctctctaacaacccccgactcttcagagttgggagcgttggtttgcctggaaccagcttcctctttgcctgtacttctgggctgagccaagggttgacaagaggggaaagccattcagctgcggggtcccaacaaaaagttggttgaccctgtagccatgaggggaactctgtaggtcagccaaaggttcagtgggtcggtccaggggtccttggtagaagttgttagttgaactcatttggggttccatttgtaagaccatctatagcttgatggcatcgatcctggaggaaacaaatttgacaaggagtttaaaaatacgggcctgaaggcgagtaatagcaagatggctgttacgggacctagaaaggggagaagccatgttgcccagctccagaggttggtataagagtttgaaaggcgttgtctgatttcagaagccttttcctgtaaacgccaggtggcgtctcatactatccctcactggttagtgtaaaagcaacattcttcccctaagaaggtgcatagtcctcctttctcagcagtgaggaagtctaggccttggcggttttggagagtcactgctgccaaagagtctatctgggattgtagaggaaggatagattttgttatttcttgcaagctgtctgagaaatcctttgagagtgtgtggtagtaggataatgaagtagataaatctgctattccaattcctgtagcagtggccattcctaaccctataagtaggggtattagttgtatggccctgtgctgatggacttgagctttgaaggggcactgatagagtctgatttcctggggcaatgttaatgttgggacttaggaagactaaggtgtaggtgcctgtccagttggtggggaggcagatacaggttgaagttccacttaagaagaatatgccttggctgggtagacagaaatttaccctggcttttaaaggaatagggtacactgttttttctttactacttctatctctgtgtctctctttgtctcttcctctctctctctctctctctctctctctttctctctttctgacttcttttctgtctcttcctctctctgtttctctctttctgactccctctttgtctgtctcttcctctctctctctttcctctctgctggtctttccctgcctctgccagctgcttatgctgctgttctcccctctcctttcagcagtgtaagactgccacctccttgggtttttgcactgtgtgcaataactccgtgatttccttatggtatttaatggggttcacccagaggttaggaactccctttctttccatattgcatcatgggcacgaaggattagataagcatacttgttatctatacacatttattctttttccctttcccagttctaaggcttgggtaagtgccactagttctgctatctggccgctggtccctgggggaggaggcttactttcaagtactgttacatcactaactatggcataacctgccctttgtatcccattcttcacagatgaactgccatcagtgtataggttaaggtcaggattagctaaggggacttctaagagatcctcttgggtggcataagtctggactataatttgttggcagtcatgctcgattggttccccatcctctgggagaaaagtggcagggttgagagccgcacagatgcatatttgaagcatcggtccctcaaggagtagcacctggtatccaagcaggcggttgtctgatagccataaacttcctttggcacctagtgtgccatttacatcatgagtagtccagacagtgagatcctttcctagtattattttgatagcctctgatataagacggccactgctgcaactacacgtaaacagtgaggctagccttttgccactggttgtagggttgtcccgtgagtctgagtaaggacttcaagagctattcctgctctctctctgacgtataaagagaagttttgtcttgtgggaaggcttaaggctggagcttgtactagggcctgctttaaggatttgaaggctgtttctgcctctggttcccattctactagatgagtatttgccctctgggtctcctttattagagtatagagtggcctggccatcttgctgtatccagggatctgtagtcggcaaaagctggtgattccaaggaacccccgcaactgttttaatgtcttagggcgaggataagccagtataggctgtattcgtttcttgctgagggccctggttcctctggctaagattaggcctagatatttgacttgttgtaggcagagctgggcctttgatttagatgccttgtacccttgattagctagaaagttcaagagatctcgagtagcttgctggcatgaggcttccgaactggcagccaaaagtaaatcatccacaaactgaaggaccagagtgcctggacttgagaagtggcctagatcttggaccagtgcctgaccaaacaggtgagggctatccctgaacctttggggcaagactgtccacgtaagttgggacgtgtgatctgtaggatcctcaaaggcaaagagaaactgggagtcagagtgcaggggaatacagaagaaggcatccttgaggtccagaactgtgaacgattctgcttcctttggtatttgagagaacagggtatagggcttgggtacaactggatatagaggaattactgcttcattgatgagtctaagatcttgcactagtctccactgactggtttttgtactcctagaattggggtgttgcagggactgctgcattttcttactaggccttgagcttttaaatgtctaacaatatcctgtaatcctttatgagcttcaggccttaagggatattgcctttgataagaaaaagtggtgggatcttttagccttatttggagtgggtgggcatattttgcccttctgaattgtccttccaatgcccaggcttcagggttgattccctcctcaagcaggggacaacaaattggtaacttgttccccatattcatgtagataatagctccagctttggctaatatgttcctccctaataagggtatgggactttcaggcataacaagaaaggcatgtgaaaagagcaaagtctcccaattaccactgagaaggtgggagaaatacctggttacagactgtcccaggattcctcggatggtaatggaccttgaggacagctgtccgggacaggagattaacactgagcctcggatggtaacggaccttgaggacagccgtctgggacaggagattaacactgataaagctgtaccggtgtccaggaggaagtcaatttcctggccctcaatggttaaacatacccggggttcagtgagggtgatgacatgagctggcgcttgccccgggcaccctcagtcctgttggatcatctggttgggggcttctggcccagagaacctttgtcctttggggcagtgcaccttccagtgattgccttggcatagtggacatgggcgagggggcagcttgtttctcgttggacaatcttttttaaagtgtccttgcaaaccaaattgataacaagcctaaccgggtgattggcctgctccattttctgtcctctctgaaccaccaaggtctgtttgtctgagggccatgactaaggctgtagcctttctcttatctcacttttccttttcagcctgttcttctcgatccctactgtagaacacggaggttgccaggtttaataatgcgtccaaattttgttcaggggccagggctagcttttggagctttatcctgatatctgcagctgactgggtaataaacttatcttttaggatcaattgaccctcgagtgagtcgggtaacagggaagtatattttcttaaggcctcctgtagccgctcgaggaaggcggtaggattttcttcctttccctgagttatggtggatgtcattgaataattcatgggctttttcctaattctgcttagtccttctaggacacaggttaacagatgttcacgactccagtccccatgatctgagtcgaggacccggtggggatccacactgagaatggcttgctgaccagtagggaatttgtccctttcttcgactgtcattctgtcatttacgtgactgagatgccaggtatctccaaactcttgggctgcaactaaagctgcattcttttcattaaaggccagggtttgatctaacaatagcatgacatctctccaagtgaggttgaaggtttgccctagaccctataggacatctatatacctatcaggatcatctgaaaacttccccaggtctactgtcatctgctttaaatcagagagagagagaaggggacatgtacctgggttgggccaaattcccctccccctacagcttgaaggggacataaccgatagcctgggagtttttgtggtcctttggagatttctttgcttgtttccttctgggtggtggagattagaggaggcttatcattaataggaaggggagctgtaggaaggctaggatatgggggtaagctgagaggtcctcctccccaatattgcaagctttgcatagttgtggattatccttcagtgaaaagaaagcttggacataaggtatttcactccgtttgtcttccctcttacagaaaaggtcaagctgcaggatagtattgtaatttatatttccctcaggtggccatttttgaccatcagagagagaatattggggccaggccatagtgcagaaaaaaatgagccgcctctttttcagggtttgcaggtcaaattggtcccagtggcttaggatgcatttcaagggtgagcctgttgatgcctgtgtgttccccatctgaaagaaaaaacctcctgtggttttggtttgttccccgctaccctctcaaccccgagttatcgtggctttaaaaaccggaccattgtcgctttggctggcctatagaattcccctctggaggacactaccactgcagggccccttctttacccctatccagcaggaagtagctagagtgggcatcgcccaattcctagcagcagttggggtgtcctgtttagaggggggattgagaggtgaagccagctggacttctgggttgggtggggacttggagaactttgatatcttacaagaggattgtaaaatgcgccagtcagcgctctgtaaaaatgcaccagtcagtgctctgtggctagctagaggtttgtaaaatggaccagtcagcaccctgtaaaatggaccaatcagtgccctgtaaaatggaccaatcagcaggacatggagggggacaaataagggaataaaagctggccaccccagccagcagcggcaacctggtcgggtctccttccacaccgtagaagctttgttctttcgctcttcacagtaaatcttgctgctgctcactctttgggtccgtgccacctttaagagctgtaacactcaccacgaagttctgcggcttcattcttgaagtcagcgagtccatgaacccactggaaggaaccaactccgcacacataaagatgaggaaaattgttcttttggttttgacgactttactatgtgcagttagcattgctgaatgtggtggtctctgagagcagtgtaattctgatgatacatacaggcatttgtctattaaaaatgggatgaaattgaagatggtttattgccttttatatcatcttttggatttgtgataaagacaaacctttgaaaatgtttgtagttattaatatgaccagaaactgaaactgtttcagtttggcacaggagagattagtagtagtattgctgttagtcattatgtaagagatgataatgtttggatttaaccttcgattgtttgtaatatgatattcctaacattcctgactaaaaggctagtgaataaaaaataatgcatttacaactatattaaaactgcattgcagaaagcatatgaaatttcaagtttaggaaatttcctgctttgaaacttttttcttcacacttcacgctttcagcacttatgtgtgatgcatctagaagatgcatctggttttgccaaatacagtactgcacctgaattacattttcggtatttattcagtatttattttcagtctttatttcctaaaggttaattttctttaaaaccttagcaatttttcatccagcccttttcatgttataatcatttgcttgtgtgattttaccacacactgtatccaactaggtagcattactggaaaacttatttgattttttgccaaatgggagatggctggaatattttttgtagtagagaatatgtggagaaaatgctttaattgatccctgaccaaatacagcaagaaaaacttaagtaaaccttaataagcaggcaatagaagttaaggtgataatattctgactttggatataaacactcaaacatgagctctgctactggaaagtaaaaaataaaattagtgttaagttgagatgagtcaacttgttgatcatctgcctagcctactgtgataggaatggttgtaagacttggaagtttttccttcctaagttgataagcatttgagtggaaagaaagtcacttttctcttgatgttttttcactttggaattttcagactgtttaaagtcttttaaaattttctgagtcacttctttattttctaggctgctcatattaagagaagtattgcctttttatcctttaaaatgtaataccctcatctttagtggttgtccctgcttaggttatattgcttagaacataacgtttatgtttggttaaaatttcactgagccaccgggcacggaggctcacacctgtaatcccagcactttgggaggcaaggcaggtggatcacctgaggtcgggagttcgagaccagcctgatgaacatggagaaaccctgtctctgctaaaaatacaaaattagccaggcgtggtggcacatgcctgtaatcccagctacttgggaggctgaggcaggagaattgcttgaacctgggaggcggaggttgtggtgagctgagatcgtgccattgtactccaacctgggcaatgagaatgaaattccatcttaaggaaaaaaaaaaaattcagcttctgaaaagcactagggactaagcaagttgactatgtaggctttattattagatttgtgtgtttgtattataaggaaaacaaatttttagtaaaatataatgtattttgttcattgtttagttcattgctggattgctggtctctagaacttttcctggtacagaataaacatataataagtattttttaaatgatgtacttaattttattaccctaccatgcttgaaactgtgactttatattatagtttcctttagcaaaaggtgaaactatagtcaatatatagattataagtaaatttttaaaaatcttcatttaccgtaatagcatcattttacatgtatacctgcttttcagatttgtaactcatttggtttaagtttggggcatttggatgtttctgatgttgaatgataaaatgtaggcttaaaaaaattatccttcgagttttatgctgctgtattgtgtatagcctctctttcataatgatacttggactgtgatggtataaagtgactcttattttaatctgaatgcattcgagatatttcaagttcttgaaagactgttacacttcttgggatggtcatcatcatatttttgaagcagagatcctaaaacttaaaaacctcagagaaagtccagcatctattttcaactccttgttttttaaagctttagaacttgactttgtactctttggtgaggtgacaaaacaaattcagatttttttttttaactgaggacttcaaaaaatttttgtgatgtttatagtattttgtctctttttagaaagtcaaatttgttgaggtatagtttacactcagtgaaattcacccattttagatgtatggttctgtgaattttgacaaacattactgaaatggctaaagtcgactgctatctagcagacagtgctaaaatagcaacttagcagttaaaatgtcatctttttatcactttgggaagattctcattggtatctttccatgggtacaataagagaccagcagtgtggcaggctttcactgtctttttccctcaactctacctcacataaaggtgagattttcctattttcttaggaaagtcagctagtggggtagggtgataagctaggacgggttccttgtgattttatttaaaggttggtctacttaatgctgggggaaaaaagaagtgtagtgatacaacatgttgcctacctttacattttcaaaataattttaaggaaataaactgctgtttgattcgaaatgattctttgtgttatggcagttaagattttattaacaccagggctcgagaatgcctgttaaaaatctaggtgttgctccttctcagctaccaactatatctttttttaagggccatattagtaacatatgttgggaacttttgccatgttaccaattatgctttttcagttttttttttgagacttttctactcaaaaaggttctgagtatttagaaagtactccaattgtaatcattctttttattgcttctggaatgtagcagggctccatgttcttccccatttcttgacagtttcttaatatcttggaacattgttgtagagtataatgtcctatctgtgttgaaatacttggctttggcatttaagtgctatcatgcatttatttgagaacagtttatggctcattcagacattgtggataagagcattgacttaggtgtcaggcagaataaatttaatctgactctgccatttagtaactatatgaacatggataagttaattataactgctctagctgttttgtcatctgccaaatggggaaaataatcatatgtatcttccaggattgttgtaaggattaaagagataatccttgggaaatattttattgcatgtagtaaacactcagagcatgttagctgctcctgtcatcctcactcagcagaagtttttttttgttttcatcttttttttttttttggagatagtcttgctctgtccccaggctggagtgcagtggtgcagtctcagctcactgcaacctccgcctcctgggttcaagcagttctcctgcgacagcctgtggagtagctgggattacaggtatgtaccaccatgcccagctaattgttatagttttttagcagagatggggttttaccatgttggccaggctggtctcgacctcctgacctcaagtgatctgtctgccccggcctcccaaagtgttggaattacaggcttgagccaccgtgtctgtcctcttttcatcccagaccatgcttggtgttgtggatacaaagtgaattctaaagaaggcctccccatcttaagaagtttcatagttcaagaagggaagacaggcatgtaaacagataaatgcaataagttgtgacatatgctatatcagaaacaaatgaaaagaataaaggagttagtatcataagcctggggaggtcagggaaagacatttctatccctgacttatttttgatgtgtgaatagtagtttcctgttgacttatttgacagatatttactgaatacactttacttttgccaggcactattttaggtgctgggaataccactggtttcatggaagacaatttttccatggactggtgggcagggggtggaggggatggttttgggatgatccaagcacattacatttattgtgcactttatttctattattactacattgtaatatataatgaaataattatataactcaccataatgtggaatcagtgggagccctgagcttgttctttcacagctagactgtcccatatgggagtgatgggagacagtggcagatcatcaggcattagattctcataaggagcgtgcaacctagatcacaatagggttcaggcttctatgagaatctaatgccaccgctgattttacaggaggcggagctcaggtggtaatgcaagcagtggggagcagctgtaaacacagatgaagcttttgagcccctcctgctgtgaggccaggttcctaacaggctatggaattggcttgtagcctaggggttggggacccctgctgtataggagttgtgaaaggaaattaaatcttggggccctcaatccctaaactaaaggaaaaagtcaagtggggaactgcttagggctaacctgccttccattctattcaaaatcatccctctgctcactgagatagatacatatctgattgcctcccttggaaaggctaatcagaaatgcagccattggtctctcacctaactttaacctggaagctccctccctgatttgagtcttcttgcctttgctttaagttgtcctgcctttccagaccaaaccaatgtacttcttacatatattgatcggtgtctcaagtctccccgaaatgtataaaactaaactttgccccggccaccttgagcacatgttgtcaggacttcctgaggctgtgtcatgggagcgtcctcaacctaggcaaaataaaataaacttttttttttttttttttttttttttttttgaggtggagtctcactgtcatccaggctggagtgcagtggcacgatctcggctcactgcaacctccacctcccgggttcaagtgattctcttccctcagcctcctgagtagctgggattacaggcacatgccaccacacctggctaattcttgtatttttagtacagatggggtttcaccatgttggccaggctggtcttgaattcctgacctcaactgatccgcttgccttggcctgccacagtgctgggattacaggcgtgagccactgtgcccagcctgcaaaataaactttctaaattaactgagacctgtctcaaattttcagggttcacagagtaaagaagacagataatgtctctactctcaggagctttcagtctagtgggaaaagatcagtagtaaacaaaaatatcagagacaagtgctatgaaagagactaaaaggtgatgtaataaggagagactgaggggttgctttacattgcattgtcagggaaggatcctttgagtaggtaacctttgagatcttcctacaaaagagtcagccatggcaaagattagagggatgagcattctaccatagggaaccatgttacacaaagccccttgagctcttggagtatttaaggagcagacaggtcagtgtggctggaatgtaatgggcaaggggtagagtggtgcaaaatgagatcagagatagtcagggtctaaatcatttaaggctttgtaagccagagtaaggagtttctaggtgtaaacgaaagccagtggagggctgtagcagggggaacgccttttgaaaaagattactgtcgctgccgtgtgaagactggattctgaggataagggtaacaagggtggaatttaggagacctctggaggctgctgtattagtataggtgagaggtggtggtggcttgggccagagagtaaattagtggagaaaaataggttgatttgggctgtgttttgaagattgaatcaacaggacttgctgactgcatgtgatcaaggcaaatcctaggacttgagcacttagaagattggtggtgcagtttactgaaatggataagactggaggggagagcaagtttggtgggacaatcatgagttctgttttgttaagtttgaggtgccaattagacatctatgtagagatttcatacttggtggtaggtaatagtagtactgtggaattcagtgagagtttagggccagggataaatatttaggagtaactgatgtatgtctgctagatttggtaagtgaccttgacaagtgtggtcttagtgggaatggaaactagattgtagagaagtatcaggtaaaggatggaatgagaatatgaagttggggaatggagacagtgacaatagactcctaagaaattttcctgtgaaaagggaagcagaaaaatggttgtgaccacaagtaaggcccagattcgaatcttacttttatacttgcctggatacagttattatttgagtgcattctaaagtttaagatattttatgtaccctacgcagtcctctctacaaatattgaggtagatgatacttttccttctgtgacatgagagtccttagtttagggaagttaagtgacagtccaggtagaacctgtatttggactcactcactttgactgtagatgcagagtatttttcattataccaaaatgctccttaaacttttattatcttggtcaaatccaatctaccttagagtttttggaaggattacaagatgagagatgtgaaaattctttgcaagctctaaattgttacacaaatatagaaattcctttctttaaaatgccccattatgtcttctttgtgcatgactttttaggatatagtaggtagtggccttgaagttcattttggatcatagcctatctcattagagcccagagaatgtaagcagattgttaaagacatagaacctgtcaaagctatctatggcatatgaatcgaacatggatcatgtttaattgaacatttttaccctggtttaatgatcttgtctccttcgcatgttccatgaggtctcataatcctccccaaaggagctcttcccaaaaataattccttctaaaaatgtggaagggctgggcacagtggctcatgcctgtaatcccagcactttgggatgctgaggcaggtggatcacctgaggtcaggaattggagcctggccaacatagtgaaaccccgtctctactgaaaaaaaaaaaaaaaaaaaaaaaattagccaggcgtggtggtgggtgcctgtgcctgtactcccagttactcaggagactgtcggaagaattgcttgaagcgagaggtggaggttgcagtgagccaagatcgtgtcactgcattccagtctgggtgacagagcaagactccatctcaaaaacaaaacaaaacaaaacaaaacaacaacaacaaaagtggaagtaattttaaaaaaactgcaaaaaagccaatcctgaaaggttacaaactatatcattccaaaagccgtggaaaacagtaacgtgagtcctcaaagaattaaaaatagaattaccctgtgatccagcaattctacttctgggtatatactcaaaagaactgaaagcagcagggtcttgaaggggtatttctacacccatgctcatagcagcattattgacaaatgctggaagtagtccagctgtccccaaatggaggaacagataagcaaaatgttactggtatacatacaaaatggaatctttttttttttttgggagacagagtcttgcactgttgcccgggctgaagtgcaatggtgtgatcttggctcaccgcaacctctgcctcccgggttcaagcgattctcctgcctcagcctccctagtggctgggattacaggtgcccgccacgacgcccagctaattttttgtatttttagtagagacggggtttcactgtgttggccaggctggtcttgaactcctgacctcgtgatccacctgccttggcctcccaaagtgctgggattacagatgtgagccaccgcacccggccacaaagtggaatcttatttcacctttcaaaggagagaaattctgacatgctacaacctggatgaatcttgagaacaatacactaagtgaaactggccagtgaaaccagtcacaaaaagacaaatactgtatgattccactttaatgaggtacttagagtagtacaaatcacagagacagaaagaatggtaattgccaggggttgaggagaagagggaatggagagttactgtttaacgggtatagtttcagttttgcaagttgtagagttctagagctagatggttgtaagggtcacacaacaatataaatgtactcagtaacattaactgtgcacttaaagataatttagatggtaaattttatgttatgtgtattttactatgccatcatgcctggctaatttttttgttttgttttgattttctggtgacagggtctcactctgtcgcccaggctggaatgcggtggcacgatcacggctcactgtagcctcgacttcctggactcaagtgatcctcccatcacagcctcccaagtagctgggactacaggtgtacgccaccactcctggctaatttttactaactcactcccttttctgcctcctttcagtattcattcattcatttaacagataatgactgaacaactgttaggtattattaggtattatatattattcctgggagcagactattaagagtcactctataccatcacagtcccaagtgtcattatgaaagaggggctatacacaatacagcagtataaaactgtaaacagattttttaagcctacattttatcagtcaacatcagaaacatccaaataccgcaaactttctttttttttttttttgagacagtttcactcttgtcacccaggctggagtgcaatggcgcgatcttggctcactgcagcctccgcctcctgggttcaagtgattctcctgcctcagtctcccgagtagttgggattataggcatgcgccaccatgcctggttaatttttgtatagtagagacggggtttcactatatgttggccaggctggtcttgaactactgcccccaggtgatccacccgtcttggcctcccaaagtgctgggattacaggtgtgaaccaccacacctggccaaatgtcccaaacttaaaccaaatggactataaatcaattccctgaaaaactggtatacatgtaaaatgatgctattacattacagtaaatgatctttaaaaatttatttatagtctatatgttgactacagttttaccttttgctaaaggaaactataaagtcgcagtttcaagtatggtagggtaataaaactaagtacatcatttaaaaatacttattaaatgtttatctttgccaggcaatgttctagagaccagcagtccagcaatgaactaaacaaaatgaacaaaatacattatattttactaaaaatttgttttccatataatacaaatacacaaattaataataaagcctacgttaatgcattgtactaatctgtcttggtgagaagttcaatttatgaagatacagtagaaatgattttaatttcttttttttcccctagagaatgtgatgtttattcattaatttattcattcactcaagaaatcttcattgaatgtctactgtgtatcagttgcagtttgggattcagcagtgaacaaaacataaatgcttgcccttatggaacctacaatctgttggttttaaaaaaaatctctcctggattagtgttagatttctagaccagaagtgtcaattaagtttaacaatttcttcaggcagagaaaattgcttttgaggtatcacttaagcaaccttagtgcttctaagcaacataactgccttttgaggtatgaactgaagggaagtgaagtataagcaatagagttgggattttttttttcttgagacggagtttcattcttgtcacccaggctggagtgcaatggcaccatcttggctcactgcaacctctgcttcctgggttcaggggattctcctgcctcaccctcctgagtagctgggattacaggtgcatgccaccacagccagctaatttttgtatttttagtagagacggggtttcaccatcttggccaggctagtctcgaactcctgccctcaggttatctgcctgtgttggcctcccaaagtgctgggattacaggtgtgaaccaccacccctggctgagttgggattttttaattttccatagatttcccattaggaaggcaacctggtgtgacggtatttgagagtcaagagatctgggttctaatcctcatttcatcatgtaatactttgtgtgtttcagaattcttcacagtgatccttgcttgtcctcactcctttgtacagcttttttttttaaaggattttatccactgttgtgccagtaactatgctagtgtcactttttatgctagtaatagttctattttcaagttcattctgtttcctgagtttaaatttgtgtttcctagtgccaactgaacatttctgtcatttcatactccatttccaaaaagaaactcattattgttccgctttgaatttgcttcccttctttatgatcttttcatctcggcaaatagcattgctatgtacccagctgcctaaggaaaaaccctgagtcgtcatctttctatatttcattgaccctcattcagtcattcaaatattgcttctcttgagtatatcttgaatctatcctttatgctctaatttctgaacagaactgacactctttgtcctgcatgttctactttctttgcacagaataccttctctttagttggcaaactccaactcattggttaatgtccagctcaaatgttaactcctgtaaaaccttcaccatttcttttaggcagaattagtgattcctgagtctttgctcttagagcttgtaggtcaagtgtgtactgtatttctgttctttcacagttttttattcgattgtctgtagaatagactttattattgtcaaggggaagacagccttagcaaaattttgaaataagtcattgtgtgcctaagcataaattagtttgtgagaacatactttttaaagagaaatgtagtagggtgtatagtatgtttgagtatctttttccaataagcattgcatgtataatcattcactcattgcatgtataattattatttaaatattctttattaattaaagacctctctggtctctccagaagtcttccatggctgccctcatccccaccctccagaggaatctgaagccacatgtgctccctggcacccacagcccctgcctctcccagagcagcagtacctgagcctcagtggattccaagaatcgaaacccttggtctgctgcccctccctcccagaatgtttctgtctcattcttacctactcaaggccctttcagtagccccttggggtattctcttcctatgcaccagggcaacttctgaactcaccactttcctggaaagacccccaacatcttcggggagcggtcccctacaggcctggctgcagatgcccgtgcacacaggaacactgtttaaaaaaaaaaaaaaaggatactggtgggaggggctttgtaactgaggaggtttaggaattgaaaactcatgtgtattttctttctttcctcattgtcacttcccagctacccagagatattgaaggacagctgagctcccctacttgctgaaaatgcattgatcaaatttgtgtaggatttccacatctatttatgggctcagaacactttctagttacttgttccttcctatcagctgttttctgtttgtgttagattttttttttttctgagatgaagtctcgctcttgtcccccaggctggagtgcagtggcgcgatctcagctcactgcaacttccgcctcccggtttcaagtgattctcctgcctcagcctcccaagcagctgggattgcaggcacctgccaccacgcccggctaatttttgtatttttagtagagatggggtttcaccatgttgatcaggctggtctcgaactcctgacctcaaatccgcccacctcagcctttcaaagtgctgggattacaggcatgaacaccacacctggcctcatgcagagctttaaaaaaaaatgttaacacggctgggcgcagtggctcacgcctgtgatcccagcactttgagaggccgaggtgggtggatcacttgaggtcaggagttccagaccagcctggccaatatggtgaaaccccgtctctactaaagatacaaaaaattagccgggcgtggtggtgggcgcctgtaatcccagctactccggaggctgaagcaggagaattgcttgaacctgggaggcggaggttacagtgagccaagatggcgccattgcactctaggctgggcgacagggcaagactccgtcttaaaaaaaacaaaaataaataaataaataaaagaaagctctgcatgatacaatacagtattcatagaattttggccagtgaaaattttctgtgttagtagaataaaaatgaactcttctgatttacagatattgatagtctggtctttccatcttccatcttgactctcttttactgttaaaaaatcttagaacaaagtaaagccagttccaaatacttttcctagttctggactgtttttttttttttttttttttttaacaagacagtcatttgtccgagtacaacagaacagttcattgttaatactttggttctgtgtgctttaaatcttttttccccctctacccataatctccctttgtaaactttgaatgttttgtcagtagatcatcatcataggtaaagatactgcacatcaagactgttggtcttccccattcttcctggaatatactggaagctgtcaggactgaggttaatttctccattcctagaactatattgtaataaatattggaatactggcttttgtgtatttttttgtgttacttagtttttggagaaaggaaatatttctagactttcttgacttgtcataactttttattgttaaagaatatagtttaaaaattattaaatattagggagccttctgcttcattgggactttaaggaatgatagccataggaagggatatatcttatattatcataaagcttttaaaaaataatatatttattacaattttttaaatttattttttatttttagaaacagggtcttactctattgcccaggctggtctcaaacttctgggctcaagtgattctcctgcattgacctcccaaagtgttgggattacaggcatgagccaatgagctcagccagttgttttttttttttttttttttttttgagtgtttttttttgataacttctaattattttcttatcctatccctaatagttggcatcagttaatgaactagttttgattccccaaaagccctctcttaatctcctgtattttgctttactttctatctcttttgtttatttcttagagaaatgtcatttatgtaatcagttcttattttgtaatactgggccgtaagaaagcagggaaaagcacaaaacttggcacatagtaaattttgttgcttgaacaaattaatgaatgaatgaacttgcagcataagagtgcaccctatttagtggtactgtttaattatttcatgatccttagccaagaacgaaggcataccggaagggaacacacaacaaaaaaaggtgagggaaaagtgttaatatatcatttaagtgactgtattacaacaatattacattatgttgtcactcctgaacatgagatgtgattctcatttacatttcaagctaatttggttagctgggggaattcttaggcatctcaagagataattagatataccccttaggacctcacataattataatcactattaggggtattaaagtttttttttttttttaaacaaaaacttagggggaaaagttgtgattggtaagcgtttttttctcttagtgttctcagaatcttaggagatacgttatttgaaacagttgttcaggccgggcgcggtggctcacacctgtaatctcagcactttgggaggccaaggcgagtggatcatgaggtcaggagatcgagaccatcctgactaacacggtgaaacctcgtctctactaaaaatacaaaaaattagctgggcgcggtggcgggcgcctgtagtcccagctacacgggaagctgaggcaggagaatggtgtgaacccaggaggcggagcttgcagtgagccgagatagtgctactgcactccagcctgggtgacagagcaagactctgtctcaaaaaaaaaaaacaaaaaaaaccaaacaacacttgttcaatcaattctactttagtagaaaccagctctctatattgttctctaacattttgttatcttggcttttagatgaaatctttagcatcagttgggctcattagcacctctactaagagaagcaagcacagaatggaagggagagcaaatcataactttttaaaaagttatttctccttagttaaaagtttggaagagaagcaagttgaaactgtaggctgattatgtaaaagtttaagttatcagctctcttatatgtaatgcaggttgattataagtttctgggctgttgtgacaacaagaaaattatgtttattattaaaaacttgtttatactgcatatatgtgaagggcattatactgtgacaaagctaacaataaaacatacattttaatatttcagattccttattttatttgtttgtttagagacagggtcttaatgttgcccaggctggagtgcagtgatgcgatcatggcttactgaagcctcaaactcctgggctcaagcgatcctcctccctcactctcccaagtagctgggactacaggcatgtgacactgcgtggctgatgttttgtagaggcggcatcttattatgttgtctaggctggtctcgaactttctcccgcttcagcctcccaaagttctgggagccactgtgctgggccagattcctttttttaaattgtgggaaaatacacgtaacataaaatttactatattaaccattttaaagggtgcagttcagtggcattaggtatattcacaatgttgtacaatcatcacactacctagttccagaactactttatgatcccaatcagaaactatgcgcattatacaataactcctcattgttctcttctcccagttcctggtaagttctattacactttctgacgctatcagtttgcctgttctaagtatcatataaatggaatcatatagcatttgtctttttgtgtctggtttatttcacatagcgtacatgttatagcatatatcaaaattttattccttttggccgggttcggtggctcatccctgtaatcccagccctttaggaggccgagctgggcagatcacctgaggtcaggagtttgagaccagcctggccaacatggtgaaaccctgtctttactaaaaatacaaaaattagccgggcgtggtggctggcgtgtgcttgtaatcccagctacttgagaggctgaggcaggagaatcgcgggaacccgcggggcagaggttgcagtgagccgagatcgtgcagctgcactctagcctgcatgatagagtgagacttggtctgaaaacaaaaacaaaaacaaaaaacaccaagtttatttaaaaaaaaaaaaagcttagactttttttgagcatttttatgttatagaaaaattgagtgaaaactagagttcccatatatctacccgctgcttggtaccaccagtttcccgttactaacatcttgcattagtgtggtacatttgttacagtttatgaactgatgttaatacatttttaattaaattgcatagttttgcattagggttcactctgtgtgttgtacattctgtgggttttgacacatgtatgaaaggtatccactattacagttatcacagaatagtttcactgccctaaatactcagtgtttcatctactcatccctaaatccctgccaaccactgatctttttactgtctccttagttttgtcttttccaaaatgtcatataattggagtcatataatatgtaatcttttcagattgatttctttccattggcaatatacacttaaggttctccatggcttttcatggcttcatagctcatttctttttattgctgaatagtacactgttgtatggatgtgtaccacagtttgtttattcattgacctattaaagactatcttggttacttcccagttttggcagttatgaataaagctactataaacacttgtatgcaagtttttgtgtgcacataggtttttaactcatttgggtaaattgtatgcctttctaacttttgtatttatgcatacacttttatatagttgtaaaacttagttttatctagttgtaataattttgttttatgttttaagataacataaacaatcttcatgctgtttttgaatgctccctaatattaaaatgagaaaatgaatttattatgtactttatttttctgttactcattaattgcttccattttcccctgccttaataaatacttttgtgtgtgtagtttttcccttttgtattgctttaggataaaattcaaggagaaacagtaaagagtacaattattgggatcaaggccattttagataattcttgatatgcagtaaagtttcacaatgggaaaccttgttagatttacagtggaggtttgctgcagtttattttgtggctttctagacaagagtagcaccagttatctgtgtaattttacttagaaaaaaacaaagaacttttcactggttctttgaagtggtagtaagcttccctactgttggttttgttcaaaaatcactgtttgctatctgaatcttcttgattacttcagaactttatgtaatagtttattgaggttcttaggtacaggtagctaacacgggcttgactttatcttactatggtacaatctaaatatatatttagtgttgttaattaaggttatcattcacagatgctcccttatatataggtggagaaactgaggcttcccttgagacagcatgctcctggatataaccgtgagagcagttaaactcttgagacctgcattctttatttttcttaattatcagtacaattattggatcagagtttcttctatcttttttgtgagtcattaaattccatttttaaaaggtatcttttgaatacatacatttacaggtttggaaaatatatttctagttaaaccatgtgttcttatttatttatttattttttattctaagttttagggtacatgtgcacaacatgcaggtttgttacatatgtgtacatgtgccatgttggtgtgctgcatctattaactaatcatttaacgttaggtatatctcttaatgctatccctcccccctccctcaaccccacaacaggccctggtgtgtgatgttccccttcctgtgtccatgtgttctcattgttcaattcccacctatgagtgagaacatgtggtgtttggttttttgtccttggcgatagtttgctgagaatgatgatttccagcttcatccatgtccctacaaaggacatgaacacatccttttttatggctgcatagtattccatggtgtatatgtgccacattttcttaattcagtctgtcattgttggacatttgggttggttccaagtctttgctattatgaatagagtgccgcaataaacatacgtttgcgtgtgtctttatagcagcatgttctctaatcctttgggtatatacccagtaatgggatggctgggtcaaatggtatttctagttctagatccttgaggaatcgccacactgacttccacaatggttgaactagtttacagtccccccaacagtgtaaaagtgttcctatttctccacattctctccagtacctgttgtttcctgactttttaatgattgccattctaactggtgtgagatggtatctcattgtggttttgatttgcatttctctgatggccagtgatgatgagcattttttcatgtgtcttttggctgcataaatgtcttcttttgagaagtgtctgttcatatcctttgcccactttttgatggggtcgtttgtttttttcttgtaaatttgtttgagttcattgtagattctggatattagccctttgtcagatgagtagattgcaaaagttttctctcattctgtaggttgcctgttcactctgatggtagtttcttttgctgtgcagaagctctttagtttaattagatcccatttgtcaattttggccattgcttttggtgttttagacatgaagtccttgcccatgcctatgtcctgaatggtattacctaggttttcttctagggtttttaggtttttaggtgtaacatttaagtctttaatccatcttgaattaatttttgtataaggtgtaaggaagggatccagtttcagctttctacatatggctagccagttttcccagcaccatttattacatagggaatcctttccccatttcttgtttttgtcaggtttgtcaaagatcagatagttgtagatatgcggcactatttctgagggctctgttctggtctatatctctgttttggtaccagtaccatgctgttttggttactgtagccttgtagtacagtttgaagtcaggtagcgtgatgcctccagctttgttcttttggcttaggattgacttggcaatgtgggctcttttttgcttccatatgaactttaaagtagttttttcgaattctgtgaagaaagtcattggtagcttgatggggatggcattgaatctataaattaccttgggcagtacggccattttcacgatactgattcttcctacccatgagcatggaatgttcttccatttgtttgtgtcctcttttacttcattgagcagcggtttgtagttctccttgaagaggtccttcacgtcccttgtaagttggattcctaggtattttattctctttgaagcaattgtgaatgggagttcactcatgatttggctctctgtttgtctgttattggtttataagaatgcttgtgatttttgcacgttgattttgtatcctgagactttgctgaagttgcctatcagcttaaggagattttgggctgagacaatggggttttctagatatacaatcatgtcatctgcaaacagggacagtttgacttcttcttttcctaattgaataccctttatttccttctcctgcctaattgccctggccagaacttccaacactatgttgaataggagtggtgagagagggcatccctgtcttatgccagttttcaaaggcaatgcttccagtttttgcccattcagtatgatattggctgtgggtttgtcataaatagctcttattattttgacatacgtcccatcaatacctaatttattgagagttttagcatgaagcattgttgaattttgtcgaaggccttttctgcatctattgagataatcatatggtttttgttgttggttctgtttgtatgctggattacgtttattgatttgcgtatgttgaaccagccttgcatcccagggatgaagcccacttgatcatggtggataagctttttgatgtgctgctggattcggtttgccagtattttattgaggatctttgcatcgatgttcatcagggatattggtctaaaattctcttttttgttttgtctctgccaggctttggtatcaggatgatgctggcctcataaaatgagttagggaggattccctctttttctattgattggaatagtttcagaaggaatggtaccagctcctccttgtacctctggtagaattcggctgtgaatccatctggtcctggactttttttggttggtaagctattaattattgcctcaatttcagagcctgttattggtttattcagagattcaacttcttcctggtttagtcttgggagggtgtatgtgtcgaggaatttatccatttctcctagattttctaatttatttgcgtagaggtgtttatagtattctctgatggtactttgtatttctgtgggattggtggtatcaccacccctgtatcacaatcccctgtatcattttttattgtgtctatttgattcttctctcttttcttctttattagtcttgctagtggtctatcaattttgttgatcttttcagaaaaccagctcctggattcattaattttttgaagggtttttttgtgtctctatccccttcagttctgctctgatcttagttatttcttgttttctgctagcttttgaatgtgtttgctcttgcttctctagttcttttaattgtgatgttagggtgtcgattttagatctttcctgctttctcttgtgggcatttagtgctataaatttccctctacacactgccttgaatgtgtcccagagaatctggtatgttgtgtctttgttctcattggtttcaaaaaacatctttatttctgccttcatttcgttatgtacccagtagtcattcaggaggaggttgttcagtttccatatagtttgtttttgattcagaaaatttggtcagaattagtgtctacacaatgggcctgttttcatgtccaccaggcattatggctgagtgaaaagccagaagggcccgattttgaacttttcttccacttaccagccatgagactgagcaacctgcacaaactgtgagccttgaattcttcagttgcagaaatgaggtgttgaatccttcaggttgttatgaagaataaatgagataatgggcacctgacagcatatatttatgttaggaatatataattattgctgtttgaaattgtagcctgtatatatttgcagagtgcccaaatacttgttcctcagatacagagtttatgtacatctcaatgtaccttttatagttgaagttgttttgttttgagagaagactgaagttatttgtggggttttttgttgttgttttttttcttacagtattgtcatgtggaaaatacatgttaattatcagggtttaggttttctgctgagtgtgtttcaaattaagtcatttccctctagaattggtagtttgttgtcttcatgttatttataatcatttccaagtttaccagatagaaaaacccttaatgaaattttggaaaagaagtacaccagtgagttgtttgctaatcttcctgtagttgtcacacagactgactgtttatatattttttggagacagagtctcactctgtcacccaggctggagtgccgtggtgtgatcttggctcactgtgacctctacctcctgggttcgagcaattcttgtgccccagcctcacgcttgggtaattttttgtatttttagtagagatggggtttcaccatgttggccaggctggtcttgaactcctgacctcaagtgatctgcccatcttggcttcccaaagtgctaggattacaggcggagccactgcgcctggcccagactgaccctttacagcatcactcctgttccttgcatattcatgcttccaatttaagattttgtccattacttattacagttgccatgtatgaaggtccctttttttgataattggttcttttctcttctacatactaaggcaggggtgggaataaatgtcacagcattaccactacttgatttgattttaatgtgttactctcttgtactcctaccttattagttttagtagtttatggctgggtgtggtagctcttgcctgtagtcccagtactttgggaggctgagatggatggattgcttgagcccaggagtttgagaccggcctgggcaacatgaagaaaccctgtctctacaaaaaaaaaaaaaaaaaaaaagttaaccaggcatggtggtatgtgcctgtggtcccagctacccgggaaactgaggtgggaggatcacttgaatctgggaaattgaggctgcagtatgagctgtgattgcactactgtgcccagcttgggtgacagagtgagaccttgtctcaaaaaaaatttttagtagtttctgtgttttgtttgtaaatttaactctgtattgagccagaaaagcacaagccctgtaatttctcattttagagaaaaacattcatttttttccttccccaaggagtttctccttcttcctatttttttctttaaagagagtttggcagaattccagagcctaaaataataaaataaaggtaattaaatgaattctccagccagcagagaccatcttatcttgaattgctagtggtttatgctggtccccctttgaccttctttcactttttggaggtggatgaacttttccactcaaaactatcttgtcagcctcacacatatttattaaaggagtgagtatgcttaagagacagattttttgtgttggttgaagaaaccattttggatatttttctctgttgtgactgggactgaatttttagattgatttatatttcctttgaagaacaaaggaatatttttctcgaaaatgagaattaacaggagcattaataacaacaaaaagtctgttaaacgtatttggggagattatttgttttcaaagaataacatttcttgtgtgagtcattatattcagcaaaatttcttttgtgataattttatgctgccataaaattttttgggaaaataactttaaaaaaacttcaaaaaattttattttagaggggcagtagtacagttggaagaggaattttaagtcaaagaacagtaaaacatgaattctttcagaagacagttgaaaatttactgtgcatcaggaacattacttagctctgaacataaaaaatgatgaaggtactgctctctgcctttatgtctttgcgtgtagtctaatgggagacacaagtaaataagaaacaatgggtcaaggtcattatagtagtagttacacaggtaggtgcatatgcttctagtctagccagtgggatgctgtgtaaattgtagctatgtggagaagccttgagatttccctctctgaaagtaaaattattgccttagggcagggttagcaaactatagctagcccctgggtatctacctgtttttgtatggctcatgagcaaagattggtgtttacatttaaaaatagtttaaacaaaataatgttttgtgatagatgaatgaaaattatatgaaatggaaatctcagtgttaataaagttttattggaacacagccacattaattcatttacatattgtctgactacttttgtgctgcactagctgagttgagttttttgtaggagagaccttttgtggcccacgaaacctaaaatatttataatctggccttttattatagaaaaagtttgttgagccttctttagagtgttatgagcactgactttttatgacctcaatcacacatgcagtttccttaaattccttattatatgtttaaaagttaaattagctgggcgcagtggctcacgccagtaatcccagcactttgggaggctgaggtgggtagatcacctgtggtcaggagttcgagaccagcctggccaacatggtgaaaccccgtctctactaaaaatacaaaaaattaaccgggcgtggtggcacatgcctgtaatcctagctactcagaaggctgaggcaggagaattgcttgaacccgggaggcagaggttgcagtgaactgagattgcatcgttgtactccagcctgggcgacaagagcgaggctccatctcaaaaaaaaaaagaagttaaattttaagtggctgtataaaggattatgatgatgcctaccttgaatggtaaacttttagatgtaacttttcagttatttctgctgaaaaatgctaaagtgatgtgctattctattgaaagccaagtgtattgatttgctaggcctgccataaagtactgcagactgggtggcttaaatgacagaaatgtattttctcacagttctggaggctagagtttcaagatcaacatgtcagcaggtttggtttctcctatcacccctctccttggcttgcaattgttgccttctctctgtcctcacagggcctttcctctgggcatgtattcctccctggtgtctcttcttacaaggacatcagtcatagtggattaggcccccccaccttgtaacttcatttaaccttgattacctctctaaagcctctatttccaaatacagtctcattgtaaagtactgggggttaggacttaacatatgaattttgagaggctacaatcagtgcacaccgccagggaatttctgatagaaacttcatttgtttacacattaagcagatcttgagggtgtgtactgtgtgacagatcctgttttacgtatgtatgtgtgtgtactatgtgacagatcctgttttagatacatatggtgaacgagacaaacaagatttgacacttaagaggctggtggtttagttgagaaagacagataattaataggaaaataaatatttgagagagtgataaggactgtgaaggaaattaaactgtgtcaagtgttagaatgattagtgtgacgggtcgtcaggtaggggcttcctgaaaaggtgaccttttacctgggagcttaaatggtaagggatagctagctagttaagttcccaggcatgtttctagggcctgatgttgatcaagattggtatggaaaaggttcaaaagaatagtgagtcaattagggagctttataagttgaggttggagaggttggtggggcaaaatcattaagggccttgtgggaattatggtaaagatttggcttccttttgcacaaggaataaaatctggagggattttagcaggggagtgacatgatttttctgttcgaaagagcactttggctgctaaataaagagaggaaactgcaggtatgagagtagaggatagggaggcctgttaggggattatcaaagtattgtaggtcagagatattggtggtttagctaagatcatggcagggaagagaaaaagaaagggaaataatttgagagatattttgggagataaaatccatgtattatttactaatggattaagtatgggggcttaggaagagggaggaatatctttggcaatagtgttgtgtaatactgtccactgtagcaagtgaacctatagtgttagcaaatcagatagatgattgctgcagcagcaagttacagaatataatttggaaaggaaaaaagccttcaatcattaaataggaaaacagaaggcaggtaaacaatgctatggtttaattatgttcaacttacaggcttataactactagacaaccaatggggaattcatagtttgccatagagaatgcctttgggacacataacctgtttggaatgaaaaagccctggagagtattgtaaaataacaaatgtcaggtttgatggaatgactatgcatgcaattaagtgattttctttagtgctagagttcttttttcttaggtaaatcattttaaaaaatggaccagactttgtgaaagtagatatgttacataaaataatgttagctaaagatgaatggaataggttaagagtgtcttcttgaaaatttgaactatatctctttcaaaaagaattttattacctctaggctggacttgactgaaaagttaaaaaaaaaaaaagacaaaatcttatgattgttgaactcccaatgaggagtcagagaattgtgagagccagggtctgttttttgtcacttttctctctctctttttttaagatagcatcttgctatctgcaggctgaagtgcagtggtgcgatcacagctgcttgcagctttgaactcttaagctcaagcgatcctcccacctcagtgtcctgagtagttgggactgcaggtgcacaccaccacgcctggcttatttttgcattttttgtagagacggagtcttgccgtgttgcccaggctggtcttgaactcctgggctcaagccatctgcccacctgggcctcccaaagtgctgggattacaggcgtgagccactgtgcccagctgccacttttttctatgtttagaaatctacctctgagccttattccccatccataaggcaggtaaccactagatggctaattttgaaagagcgagagcattcagagatgcacagtaattctaagttctgagtagaatacagtcatcctttggtatccatgggggattggttccaggacctcccatggataccaaaatctgtggatgctcaagttcctgatagagaatggcatagcgtttgcatataacctaagcacatcttcctgtatattgtaaatcatgtctagattacttgtagtaccaaatacaatgtaaatgccctgtaaatagttactatactgtattgtttagggaaaaaagtctacatgttcagaatagatgtaattttaaaaatctggataatttccatctgttgttggttgaatccacagatgcagaaccccattgatatagagggctgactgaatacaaaagcaaggcaagtgtgatagcagccttcttttactttaggattcaggatttacttctttacttcaggatcaaatatttttacttttcgattttatttttttgtttgtgaagagttgggagttgctgggtttagttatgctactctctttttaagggagaatatagcagtcctagagggatttgcaaactatagctcttggggaaacattcaaaattttccttgtgtaagaaattttatgagtctcgagggccggatgtggttgctcatgcctgtagtcccagcattttgagaggccaaggctggaggatctcttgaggtcaggggtttgagactggcctggccagcatggtgaaaccccatctctaccaaaaaaaaaaaaaaaaacaaagcagtgagccaagattgtgccactgcactccagcctgggtaacagagcgagacgctgtctaaaaaaaaaaaaaaaaaaagaaattctatgagtcttaaattacatcaagtaactttggaggcaatagtttaaattctctaaaatactgggcacattagctcatgcctgtaagcactttgggaggctgagatgggaggatcacttgaggccagtagttcaaaaccagcctgggcaacagagtgagactccagtgctacaaaaaattaaaaattagctgggtgtggtgggcacacgcctgtagtctaagcttctctggatggaggctgaggcgggaggattgcttgggcacagaaattcaaggttacagtgagctgtgattgtgccactgcactccagagtgagaccctatctctaaaaatattaaattaaaaaaaacccatacaactcaaatttccaggtactgttattaatatgagttatcttgaactgttgaaaaattaatcacttattgttgatttctatctgcatattaaagatctggcttaagagtcttgcatttgatgaataggcttttctatgttatatacaagaaaaaggagataaaataatttaacttttttattacattaaaaaacaggactctgggaattgtcactttatgatcagccttatattttttccagactgtgtcattgtgtatgcctgtgccacccaacggatgagaccactgatcatgtttagctcaaatttatgttggatagaggttataggatggtccttatctgaatgcagaaaggaatgtttattttgctgttatgttttatatgggataaaatgcttgaaaagtcttttttttttctgtatttttttttttgagacggagtcttgctgtgtcgcccaggctggagtgcagtggtacgatcttagctcactgcaagctctgcctcccaggttcatgccattctcctgtcagcctcccgagtagctgggactacaggcagccgccaccacacctggctgattttttgtatttttagtagagacagggtttcaccacgttagccagaatggtcttgatctcctgacattgtgatctgcccgtctcggcctcccaaagtgctgggattgcaggtgtgagccaccacgtccagctttttttgttgttgttgttttctgagacagagtctttggctctgtcaccctggctggttagtagttggaactataggcgcgtaccgccatgaccggctaatttttgtatttttttgtagagatggggtttcaccatgttgaccaggctgatcttgaactcctgacctcaagtgatccacctgccttggcctcccaaagtgctgggattacaggtgtgagccaccacatctgacctacttgaaaattcttaaatggtgccttatactttctggtaataaaagcactattttaatgttcggaactattttcattatttgctaaccttacaattaaaaaatccttttatatttatctttggatagatatgaggggacctcaaaaagtttgtggaaaaatggaattaaaagataaaaatacaaaatataaactttattctcaatataagctctatcgagttcaagaaaattttgtaagcaataatgctagccatttagtccactcctaaagaagtcctgtaaccatatcagtatgtgcagtcttttttttgcattattaattaaagaaaaatgggtgccctttaaatttttttttaaggttcggaaacaaaaagaagtcagaaggagccaatcagcactgtttggtggatgcccagtgatttcccattgaaaccctagcaaaactgcccatttgagaggaatgagcaggaaccttgtggtggtgaaagtcaacaagattccttgagcattccaaaaagcttttgtcatgaccttcgctcttgatgggtttgcttttgctctaactggatcactttcatctcttggtacccattgctttgtttttttctttgtcttcaggatcacactggtaaagtcatgtttcatctcctgttaaaatttttcaaagaaattccacatgctcttgttgccacttgtttaaaatttgcattgaaggcactgcttttgtctgtggccgatctgagcacaatggttttagtacccatcaagcagaaagtttgttcaacttaaatttttttgttagatttgtatacgctgaatgaatttatatgtctatggtattggctgttgtttgtgctgttaatcattggatctctacatttaggttacaaacgggatgaattttttcctaaaaaatttatgttgatagtctgctactgcaggcttcatcttcaatatcatcttgtcccttcttacaatgaattatcctatttataaactgctgatttcttaggggcattgtttctataaagttttcgtaaagcaccagtgattttaccattcttccactcaaacttcaccataaatttgatgttttgttcttgcttcaattttagcagaattcatgttgctctgttagggtgcttttcaaaccaatgtcttatccttcttagtgcctcaaactacatcttgttcagatgtgttataacaagttagtaccagcttattttggtgcaaaattttattgaaattcatgcatagtgttttcataaaacatatgttacacgaactttttgaagatcccttgtacctgtagtccaaaattcagaagttactaaagaatgtaaaataaaaataagtcttcttcctatgcttgttttcacttacccagtttccttcctcatcttcctgatggattaaaaaaaaaatgacatgatctaaaaaatatgatctattttaggtctaaggacatgtataggttgaaagtgaaaggactgaaagagttatcacatgcaaatagtaactaaaagagagctgggacagtttgctaatatcagacaagatagatttttagttaaaaactcttataaacacttaagaggctgggtgtggtggctcatgcctgtaatcccagcactttgagaggctgaggtgtgtggatcacttgaggtaaaacttcatctttactaaaaatacaaagattagccgggcatggtggtgtgtgcctgtaatcccagctactcaggaagctgaggcagaagaattgcttgaaccagggagacggaggttgcagtgagccgagatcaagccattgcactccaccctgggcgacagaacaagactgcctcaaaaacaaaaaacaaaaaacaaaaaaaaccctcttataagagacaaggaagagcattaaatagaagagtcgatacaccaagaagatataacaattataaacatgtatgtactgaacatcagagctccaaataacctgaagcaaatattgacctcattgagcaacagacagatctaaaataataactggagactttaatatgccactttcaataatgggtagaacaactagacagatctagtctagttagaagtagagtggaattataggcatggtggctcacgcctataatcctaacattttacgagactgaagtgggaggattgctggagctcaggagtttgagaccagcttggataaatagtgagaccttgtctctactaaaaattaaaaaattagctgggcatgttattgcacccctgttgtcccagctacttggaaggctgaggtgggaagaggaagattgtttgaacccaggagtttgaggttgcaatgaactgtgatcatgtccttgcactctagtctgggcaacagagtaagaccttgtctcccccactaactccactgcccccccccaaaaaataaaaaaaacaaaacaaaacaaagaaataaaagactttaacaccgctgtaaaccagttgggcctaataggcatgtatactccactcagtagctatttttctcaagtgcacatagaacattcttcaggatagaccatatactatgccataaaattagtcttaataaatttaaaaagattgaaattacacaaagtgccttttctaatcacaatggaatgaaagtggaaatatgagaatgagaactggaaaattcacaaatatgtggaaattaacacacttaaccattgggtcaaaaatcacaaggtaaattagaaaatatcgagacaaaaacacagcataccaaaacttaggggttgcaatatatatatcacttcatacaattggcttttcaaaatcagttaagacaaaaggtaaacatttatataattacaggtactctttcttttttgttgggcggtggtgggtggtacgtggatttgaattgctgtttgaggtcacttgcttccaggcttttgcatttcttttaagggggatcttccaggaacaaaatctgttttattgctttttaaaatctgagaaggtctgtttcgccttcagttttgaaatgtagttttgctagatataagattggttgacaggttttttttttcttttatttgaacactttgattatgttatctcattgttttctgtctgccattgtttctgcttagaagtcagctgctaatcttactgggttccattttaaaataatgagtcgtttggttcttgctgcttttaagattttctccttgactttgactttcaacatttttactgtgaggcatctgtctggatgtccttgcctttctctgagttaaaatacattgagtttcctggatgtgtggtttattgtcttcagtacatttgtggagttttcagccattatttctttgaattttttttcccctctaccactttttctcctctccttttagagtaccaaactgtgcctattggtgtcccacatagctctaagattttccaatttactgttgagcccccccaccctttatttttaaatttccattattgtaattttcagcttcataatttttatttggttcttttttgtagtttacatctcttcattgatctctatctgaggtgatattgtcattatgctttcctttacttttattatttatttattctttttttagagacggagtctctctctgtcgcccaggctggagtgcagtggcgcgatctcggctcactgcaaactccgcctcccgggttcacgccattctcctgcctcagcctcccgagtagctggaactacaggtgcccgccaccacgcctggctaattttttatatttttattagagatggggtttcaccgtgttagccaggatggtctcaatctcctgaccttgtgatctgcccacctcggcctcccaaagtgctgggattacaggcgtgagccaccgtgcctggcctgcgttttacatttaagtctatgatccattttgagttaatttttgtatgaagtgtgacctaaatttacaggttgaagttttttttttaagttttttgtctatggatgttcaattatttcagcaccatttattgaaaaggttatccttccattattgaattgctttggatatttgtaaacgtgtttagggaaaaactctcctcaaactgtgtttttcctctactgtcataccacagcaataatcaacacagaagaagacttctgtgatcacacacatggggttgttttccccacacaccaagcagcagacagcagcctgagtgtcctctaattcagttcgacactatctccgggtcattggtgtcagatcccacaggttgagggctccgtacctaagactactcccctaacacacacaccagtcataagtcggggcctccagaacttctgaccaaccagcttcaagttggggctcccatggccccctctttgggttcattaatttgctggactggcgcacagaactcagggaaacacttatttacgcttactagtttattataaagaatattgcaaaggttacagatgaagggacgtgcagggctacgtatgggggaaggggtgtgaaactttcatgtcctccctggatgctccacccaccaggaacctctgtgtgttcagctgtcttgaagttcactgaaccctgtcctcttgggtttttatggaagcttcatgacatcaacattcctaccccaagggcatagatgggaccctacatgagagagtcttaagacccgcaatcaaaaagtcaggggaacattaagtgtggaaagcaggcaggagaaagaggcctgcctctgaggcctaacacttccaacattataatgaaagactggaataaaggctatgggagttaaggccaggaaccatggaagaagaccaatagatatcatagcaccacaataaaataacaggcatacttgtgtgggtcatttctgggttctctgttctgttctgttactctatgtgtgtattcttttgctcataccaacacagtcttgactagctatgtagttggccttcagtgaggtctttttcgattgttttagctattttggagctttgccttccatgtaagttttataatgagtttttatctacagaaatccttgctgggatcttgataggaattgtattaacctatagaacaatttggacagaattgacatctttactgagtcagtatattgctgagaacatagtataacatattgctatttgtttagttttttttttttttttttttgagaaatggggtctcatgctgttgtccaggctggagtgcagtggtgtcatcatagttcactgcagcctcaaactcctggactcaagcagttctctcaccacattctcttcagtacctagactacacaggtgcatgccactaaactcagctaacttttaatttttttttagtgacatggtcttgttgcccaggctggtctcaaactcctggtcttgaagtgatcctcttgcctcagtctcttgtatttgtttagatttctgatttatttcatcagcattttggaatttttagcatatagggcctatacgtattttgctaagcgtatacttaaagtatttaattttctatggagcagttgtaaatggtattgttaaaaatttcagtttctgcgtgtttattgttaatatatagaaaagtaaatgatggctgggcacagtggcttatacctttaatcccagcactttgggaggccaacgtgagaggatcacttgagttcaggagttcaagaccagcctgggcaacatagcgagactccatcactacaagaaattaaaaaaaaaaaaaaaaggccaggtatggtggtgtgcacctgtaatcctaggtacttggcaggtggagggcaggaggatccgttgagcccaggagttcaaggttacagtgaggtatgattgcaccattgcactccagcctgggcaacagagtgagaccctgtgtctctagaaagaaaagagaagtgattgatatttgagtgttgatcttgaattcttcctgaactcattatttctagaatttttttttgtagatcctcagtaacattttaagatttgttttgaagtgttttatcttttttctattgtgatgtattcttatttcataatgaccaaatattgtggggttttaggatgaaggctgttgaaatccatatatcaattttatgccccatcacattgatgatttttcttatttataattttttctttagttgattctcctggattttatagataaaagagtcatataatctgcaaatagttacagtttacctttttttcccccaatttttatactcatttttttccctcatttaattatctaagacttccaagatagagttaaataatgctggtaataccattttctggctttagactcttagacttgtttgctttttttttttttcttcaaggcattgcatgctagtaggagttcattttatttcagtaggtagaatgcaaatagagtaaaacatatagtaaatttcagttctctagtgtgttctagtattggggtgttctgttttgttaaatttatgggaacctgaaggttgacataaatacttttacttcaacagttctactgttgaaagtcaatattataggccttcctgctttctgtagagtggccaatatttgatttctttgtagattttgcagcactttattttcttcattcttattttatcatacttgtcaatttagaaaacagtattgataggaatgtatattgatctcaggactttactggagagttgattttcttctttttattttaaggaggatccttaaaaattattggggaactcttatttgttgtcatttgatttcttcttctgagatgaggattacaaaacaattctaactaatggagcttttcagtttttcttttttattacgagatggggtctttgctttgttactcaggctggagtgtagtaatgccatcagagctcagtgcaacctcgaactcctgggcttaagtgatcctcccacttcagcctcttgagtagctagtactacagccatgagccacgtgccaccacacccggctaattaaaaaaatttttttttttgtagaggtgggtctcactttgttgcccaagctggtttcgaactgctgggctcaagtgatcctcctgccacagctatccaaagtgctgggattacaggtgtgagccactgtgcccgcctgagcgtgcgtttatagtctcataaatgagcaacattcctccatatatagctcctgctgatgaaaagtttggtgtgctgatatttttcttttcttttctttctttcttttttttttttgagatggagttgccaggctggagtgcagtggcatgatctcggctcactgcagcctctgcctcccgggttcgagcaattctcctgcctcagcctcctaagtagctgggattacaggcatgcgccaccacacccagctaattttttgtatttttagtagagactaattttttgtatttttagtagagggtttcaccatgttggccaggatggtctagatcttctgaccttgtgatctgcccgtctcggccccccaaagtgctgggattacaggcgtgagccactgctcctggcctctttttttttttttttttttttaagataatgtctcactctgttgcccaggctggagtgcagtggcgtgatcttggctcactgcaaactccccctcccaggctcaagtaatcctctcaccttggcctccctaagtgctgggattacaggcatgagccactgtgcctagcctagtgtggtgatatttcaagaacactttacaaagctttattctacttgctacagcttcagcatcaagatcctgactctgtagttttcttgtgatataaaccaactacatttagggttcattttcttatcttttactcaccccagtcttaattttgttgcacagtggaaagcacatgaacttcatgaggttcagaaactgggtataacacagctgcttctctttgaatcctaatgaccttgaataagttatccaaactctaatcctcaaaatggaggataataatacctcagaaggctatatttaaagatgctgtgaagtaccacatgtaaattatctggtacagaatttacctttgttgattctcagtaaatgtttgctgcctctcccctgaaacaaaatgaaatggtgtgtgtgaatagctttgtgagctttaaataatataattaaatagaagccattattctgtttcctctttcagaaatgggcattaataacataatttataatctcaatagtatcatgtgcttggcccaggtacatgcccaggtttggtattctttagatttttgtttcttcttgccctacatacttcgaaagcccttggagaaatcctacagcaaattcacatgatttatgaaaccaacagttgactaattcttactctgtttctttgactaattcttagtctatgtatccacatatatgaaactaaatccacctgttaccttctttttatggtatctgggttctcctactttgccctactcctttccccactaagaaccttgaacctcatggaatttattagctagcatataccctggtctcttcctgttttggtatttctggttggattgactgtgataatccttattggatattcaactaattgtctaaaataagcttacatatcatgagttaagaggtatagtgccaaggacttgaaaagcttgaactcctaggacttacttgctgcagtccagtttagagagaaattaatgatttctgaaaatgttcttagcactcaagagcattgagatattatcaaatattcattaaacatttgtgtgcgtagcactttctgggaacccagatggatgtaaaacgtgtatgctttggtcctgagtttcttccttccagggtagtctagttagagttcagaataactcaaaaaagttgaaatgtaagacattaagccttttgaatacaaaatattatggataatatgtgcttataaatttgaagactttgtcctgtttattttttataaagtatggtgaattttgttttcctttacctgtgaactagaaataaaaataccctgtggggtcattgtgatgtttaaatgagattggatatgtaaagcccagttataatatataaacttacatatattctttctaatacatacaattagtatttagaattctaatttctaattgtatactattttttaaattaaacaagtaatatgatacatggttaaaaaaaatgcaggcagaacaaagtaagtattcttctcacttcagacccgaggttcttccatatgttcttctgtaccaagagtatggttttaaatatttttcattaattattctagaacatatgcacttacatatacttacatatttttataacatataagtgggaacatattattcatgttgtcttgtatcctgtgtttttcatttaaaaagagatttttggctatattccctgtcaacaaatatagcttttaagtacattattcttttattattgattgctgagtattctgtcgtttggatatgtgataatataactgatattttccccagcactcctttaatgcattttttcaaaactttcatcttcttaggatcaactcactatcctgaaggtccattctcccaagaagaggggacagaaagacagatctatttgtaagaaaggcttgggtatcccatgaacgagccaacagaaaaccgattggggtgcagcaggactccagagccagatataaggctcagaaaagggcaccaactggatggtacacgaagaggtgataatgacagccaccaaggagatttggagcccattttagaggcatctgttctatcttcccatcataaaaaagtaagtcaaattggaagaatgataaacaaaggaaaaaatgtggaggaagtggcagagggaggatatggggaaaaaaaagtgggggtccattgctggagggaggatggggtggtaagaaggaaacttgcacatttgtatttttttcttctggataacttgaagtctcttatctttattattattattactactactactactactactactactactactactactactactactattgagacggagtctcgctctgtcacccaggcggagtgcagtagcatgatctcggctcgctgcaacctccgcctcctgggttcaagcgattctcctgcctcgactacaggtacatgccaccatgccaggctaatttttgtagctttagcagagatgaggttttgccatgttggccaggctggtctcgaactccttacctcaggtatcctcccacctcggcctcccaaagtgctgggattacaggcatgagtcatcgtgcccagcttcttattcttaaattagcttagatattatttgattttgattttatggttaatgtctttctgctgactcttttttttttaatttcggagacggagtcttgttctgtcgccaggctggagtgcagtggtgcgatctcagctcactgcaacctctaccttccaggttcaagtgattatcctgcctcagcctcccgagtagctgggattacaggcacctgccaccacgcctggctactttttgtatttttagtagagatggagtttcaccatgttggccaggatggtctccatctcaacctccttatccacctgcctcaacctcccagagtgctgggattatagacgtgaacgactgcgcccggcctctgctgactcttaaaagcgtacttaacctgctttatcttgttgaagggagaagacgtggtctatgggtaccatataatctaatttaaccatactttagctatgacgttatctgttatttttggagaaattcgaattgcgtgttagagctacttttccagaaattgtttacctccagtgtataccttgggagtcagtagagttctgaaagctgaagcagattttattctttcatttcctagagaaaagagcaacctttaggcaactatacctatagagcagaagtatcagaaagtcattgttacagacattttgctatgtgctttggaacttccaaattatatagcatgtgtttaatgttttattactagtacagtgtttctcaaactttgggttgtacctattagtgggttttgaaattaattgtattataaagtgttaaaaattaaagatcaaaatagaataaaaacattgaagtgtgtatatagtaagtaatacacatttaaattgttttagttttatatagttatgtatataaatatgaataataatatgaaatacatttcttactacgggttgtggttaacattttgaaagcttctactgggaacagcagctgggggaagagtgtttcaaggtagaggaaacaagctagtgccaaggtccaaaggtgggaatatgcttatcttccttgccttcccctcctgcctcccaccaccacctgcgcctgcttgtatcatctttctcttctttcatcttctcctttattttattattataattatattttttttgagatagagtctcgctctgttgcccaggctggagtgcagtggcaccatctcggctcactgcaacctccgcctcccaggttcaagtgattctcctgtctcagccttccgagtagctgggactacaggcctgcgcaaccacatccggctaatttttgtatttttagtagagacggggtttcaccatgttggccaggctggtcttaaactcctgacttcaagtgatccgccaccttggccccgcaaattgctgagattacaagcgtgagccactgtgcctggccttctttttttttttttttttttaaaggaccaaaagaaggccagtgtagttagagcatgaggaactagagggaagagtgaaatgagataaggtcagtgggccagtcagagaacagattatacaggtttattcagggaatttaaattttattttaataaaattgcctggcacgtgggcagcttttggggtgtttaaacatggagacatttgtgtttttaaaagtatgactctggttgtggaatggattatttgaagacagaatggaagagggacccaggtgttagaatatagatgagagatgatgtcacttgaaacagcatggtgggagcagcagaaatggagggaaatggttggattctggatatattttgtgggtcaaatcaacaagacttcctgatggattagctgtagagtgaggaaaatagaggaatcaaggctgattctctgagctgaactgaaatggtaaagcaagagggaagagcgggtttaaggtagggaaatcaagagtggtgttttggaaatgtgagattcaagattcttgttagatatcttagaagatatattgaggagtcagtaactgtgtcacgattagatttttatttacctctgtttcttaactatttttaaaactgaactgttattaaggaatctaaggaattttcaagcttgaaagaatttcaggctagccaatttgagattcataatggcaataaaattctgtttatcatgctacattatttttaattacgtacaaagatctaacatgtcacccagggaccatttcacccactgctctgtttggtcgccagtcttttgtctttcttttcagcaatggtgaggcagatacccttttcctcagggaagagaagtctgtggtttgttgcccttgccaataacaaaaatgttggaaagttgagtggcaaagctgtttccattggcatctttcatgtgaaccacatcaaaagatccagagtgcctctctctgttggttatcacaccagttcttcccaggttagcacctacagtcaccatgcataggttaccattgtcaaacttgatgaaattggtaatcttgccaatctccaaatctgtctgaatggtatcattcaccttgatgagggaatcagggtagcggatggtgccgggcatcatgagtcaccagatgagggattccttttgtgcccccagagatttttctcactttgtatttggcctcctcaggtgtaataggatgtacagcaaagcgacccttggtgtcatagatgagacggaaattctcccccgtcttgtcaatgctgatgacatccatgaatccagcagggtaggttatatgagttcggaccttgccatcgatcttaatgaacctctgcatgtaatcttctttacttcatctcctgtcagggcttacttaagtctgttccttaggaatatgatggggagacactctctcaacttgtggggactagtggatggacgaggagcaaacacaccggtcaatttatccagcatccaatgctttggagctgctgcccacttcagatgcttcttgggaccacgcgccatggctgcgttaggcagggaaagcaaaatatatttttagagtccagaacttgcccacccaatcacttttgtgtcagatttacattaattgatttttttatgtgttgtaatactatatatttttgtttcctgtcttcactggttgaagtggtatcttctgtaaataatatttgtgttttaatttcactaataaataattaggtggatgggatttttcccatccttatgaatgaactcatttattgccctgaaagcctctctttgattttccttttatcttgagctaatgcaaaagttagcagtatgaacaaaagaaccattttttctttcatttgttttaatagatttttctatgaggagccaaggaaagaaaggatccataggttgtggaatgttagagctgaaaaggccttccaaaatcatcttgtttgccttcctcaatttaccagtgaggaaattgtgtttttattcctaatgaaaatttgcgtgtgtatgttgtttctctttggagttgaagttgtttgctgaaaaagagaatctatatgcagtactgtcccttgctgccaatttcacggttaatgtatactacacatgggagctagaaaagaagggggatagataagagttatagagaactgcagttttgagactgaaattaaaggcaaaatattgatatcttcacatgatttgaggagttccatatgctgattttttttgttgtttggagaaaggactcatttcttaaaattttcagttttgaagttgaaacatctgaactgtagactgcaaccttctggataattctagtaatatcttaattgtgtcactttcacagacattatctcatttgcttctcaaaactatactctgaaatagagcagatatgatttctttttgaaagtagacgaaaataggatttagaaaggttagtttaggaatttgcccaaggtaacatgacaagatgagagcatgtgctaagtcttctgactgctctttctgccgaacaaagctgcctttatgagaattggcaccagattgacttaatgtttctgaattccaaactcagtccctgagttgtttatattgacctagaggttagattcaacagttggtcttgttttttggcagaagttgttgcagacctcgttttctgttaggccagggtaattttgcttctcagatgtgttctaactttgtaatgggttttctgtttattttgaaagcaaatagaacgaatatatcatagtacagtcattcacatttctaacaattttgcatcattttctttgtttttttccctcttttagaagatgcagatcttaactaaattgcttaggcttcatttaaaacattatgggagcccttaggctctcttaagttgtggagattggcattcacatgccaatcatgaataaggatcaaagtaaaataggaaaagcagcaggggtgtctcgttttttattaagtggaattctgaatttataaagtaagatttaaggtaattatgtgcattacagactgtggtatctttttgtacaagtctttatttaccgccaggtttctttaaatcaataaacctattaggagtgcccacctgatcacatattgccctttatagaaaactacaaatgccaggattttattccaggcctgctgaatcagaatctttgggtcctgggtatattttttttgtttcacaagtaagatgctcactactgtttaaaaacctccttcctggccaggcgcagtggctcacacctgtaatgtcagcactttgggaggccgaggcagacagatcatgaggtcaggagtttgagaccagcgtgtccatcatgctgaaaccccgtctctacgaaaaatacaaaaattagctgggtgtggtggcaggcacctataatcccggctactttgggaggctgaggtaggagaatcgcttgaacccaggaggtggaggttgcagtgaaccgagattgcaccgctgcactccagcctgggtgacagagtaagactctgtctcggggaaaacaaaacaaacaaaacaaaacaaaaaaaacaacctccttcccgtaggagatagttaaggaaaacaaataaacaaaacccaaaccagtgcctaattttttttgatatttatttattttaaataagtattttatttaaaatagttaatgtattaaaaacttcttagcaacatgctttgcaaagaaatcttaacacattctctgaagattgttagggaaataaagttagatttatttactgtttgttaaaggaaaatatcacatttactcacagttgactcttaaaacatttttttttgagataaattttacataccataaaattaacccatttaaagtgtacaatttaatggttttattagtcacagggttgtacaaccatcacttcattctaactttaggacatttttatcatcccctaaagaaaccttaacccatttgccatcacttcccatctctacccccaaccctaggcaacctctagtctactttctgtctctatggctttgtctaatctggacttttaaataaatggaagtgtaaggcatgtggtgttttgtatctagcttcttttacttagcataatgtttttaagtttcatcaatattgtagtatgtaccagtacttcattcctctttatgactgaataatattccattgtatggatatacaggggttctttcttatttgtggtttcacattctgtggtttcagttacccatgggtcaactgagatctgaaaatattaaatggaaaattccagaaataatttctaagttttaaactttgtgctgatctgagtggtgtgatgaaatctctcaccttccttgccctgtcctgccttacccaggaaatgaatcatccctttgtccagcatatccatgctgcagatggctggctacttagttacttagtagccttctggttataaggtcacagatcacaagaagcgtgagtgcaatacactaagatattttgagtgagagagagagcatattcacataacttttttttacagtatattgttataattgttctattttattattggttgttaatctcttactgtacctaatttataaattaaactatgttataggtatgtatatataggaaaaaacacagcatatatagggtccagcagtatccatgatttcaggcatccactaggggtcttagaacatatttcctgtggatggggggaaactactgtactatagtttatccaataatgagttgatggacactggattgtttccacttttcgactgttgcaaataatgttgctattatcattcatgtagaagcttttgtatggatgtgttttcatttctgttgggtatatactcaggagggatttttctgggttatatgttaactctgtgtttaacattttgagaaactaccagactgtttttcatagcagctgcaccagtttatattcccaccaacaatgtgtgggggttccactttctccacacccacactaactattgttactatattttgaatataactatcctagtgggtgtgaaacgatatttcatcatagtcagttagctcttgagcttttatgaagagacctgcctaggttaactttagctcaggattttaagtattaacctctttgaggaatttggaaatagaagctaaattcaaacttactataaaactcaagttgtgtcaatttcacttcaaatggtctcctcatcatctgttctgccattactttaatctaagccaccaccatcttccatctggaatacacctgacaggacttctgttttgtttcttgtgctgtccaatatattcttcgcacagttcctagagagagctcttaaaaatataaagtttattatgccattcccatttaagaaattcatttgtttcccattgcacttatggtacaatcccaaattctttctataaccttcgggatcctgcgtgatctcactgctgccctatctggccaacttcatttcaggacattcttctccatgttcattgagctgcagccgcattggccttatttcagtttcttaaacaggctgggctctttccagcttcagagccttcaaacatggctctttcctctgcctggagtgttgttacacccacacctctggctaactgctgctcatccttcagtattcaggttaagttctctacttccacttcattctatttcaagtttcccattgttatctttaattaccttttattttccattacaatgcgtatcatgacatatatttgtgtgacatatatttatgtttataaacatatatataaaacatattttataaaaatttataattgtttaataattgtttttgcttgtagactcttcagttccatggtgtaagaatcttgcctttttgttttcctctgatatcctcagagtctaacctggtctttgagtggcatataacaaatagttgttgaatgaatttatcagcaacaaatataaagttctgtgtctaagtggtttcaagtagtttctttaaagagatgtacaaaaggagcacgtggggacgtatactatttagatgcttagggaagcttaactaatctacttctttaatcttagaataaatgcattcttatcaggagaataatgatagttcacttttattgatcttttacaatggatgaagtaattttttgagctcatttgaatatgttattcgttcctaaagtagccttttagaatagatattattagaatctgttttacagatgaggaaactgaggtttcatgtgaatgtatgtgtattgtacactgggactcttgtcatggggatgatatactttttctcttcaaaggggcagggttttcttttcattggtttattgtaaaaagtgcgatatggcaatttcaatggttatttgcctttaatacattttaaaatacattatttccatagatgataagaattgtggaaaaagagaccctgtgataggtgatagtgatccattgtagttgtggaattggctctcaagtaacattaaataaaatacctgtgtgtacagttagattacgtttacctgtgcataggaatcccttccttatttgaggtgttggagaggttagtctctttggttccttatgaagcaggatggatttttagaaagagtattagaaactaaatactttaaaattgaaatctctgcagtaggaatctgtatttttcacaagttcccctatgtgattgtgaggcacacttaatgatggagagccagtgatcacagttttcttttcataatgcatatacggaagttccagaccggtaggaattacatcagggtaaagctgggcctagaggaggtgggtgtttcttcttttgtaaatgcagtatgtaatagtatgcctcttgtctcacagggttgtaatgataataacttagtgcatgtaaagcagtggtaacactgcctggtacagagaaagcacttaataaatgttagatgtagcatatcattggaggtgtatatttgtgtcaaaaagaaaagacttcctctgttatatcaaaaggacaaagttaatctttctttatttctttttgtttttattagatttgtatttatcttataaaaaatagattggtggttatctgtctccactcacttgaacatgtttattctctgcatccaaaatagtccaggaatggctggacctggtggctcatgcctgtaatcctaacaactttgagaggctgaggcgggcagatcgcttgagcccgggagtttgagaccagcctggacaacatagcgaaaccttatctctgcaaagaatacaaaaattatccgggcctggtggcatgtacctgtagtcccagctacttgggaggctgagatgggagaatcacttgagcccaggaattcgaggctgcagtgagccatgattgtgccactgcactccagccttggcaacagagtaagaccctggctcaaaacaaaaaattacacatataatgtatattccaagaatattgtatttgtgcaatatttaagtaaagaaatgaatgattaaacctgagttttagtcctggctgtactgccagccagatgtgtgactttggccaattttcttgatttctttgggtttcattttccttacattaaaatggagatgtttaactgggtgataaacatctaagattttcttatcctcaaaatttctgtggtccccgacactatattaatacttttttgtaatgggataaaaaatgagtgagggtcatagtttacttttttcataaatcaatccagttaggtatctttgcttaagcagccctgtttaatgccttctttgatcacttttatcgtgcctggttttatttagtgaagccatttctgtggactagtaaataattttgaaggaaatagtttggtaaaaggaaaaaagcagggttttgtactcagagtgacctaaatttgtatccctgatctcttctgtatcattatttttcaaacttctgtgtaagaggacccttcaaagaaaaacaacaaaaaccttattaaagtattaaacacacacacacacacacacacacacacacacgtagatatattgtaagtggatagtctgataaacttttataaactagacactcctgtataaccagcactcctggaggtctctttattacctcctcatagtaactgcctattcccactcccccaaaggtaaccattaccctgacttctaatggcatggattaattttgcttgtttttatactttatctcattgtaatcattccatatgtactcttgtatctggcttcttttgctcagtatgatgtttgtgaaattcatctgtattttctgggtatagctgtagattattcatttttattggtctgtgttatttctttatattactgtaccaatttttattttatttatatgaaggcaatctgggttgtatctaacttggggctattataaatagtgttgctatgaacattccagtacttgttttttagtgaacaaatgtatgcacttccattagctacatacctgtgagtggaattactgcgttaggaagatgatcccattctccctttaaacatcaaaaatatttaacagacctcactacatgatactcacattttaaatattagtttgaaaaaaatacatgatagaaattggctatgaattcagttcatcttaagtgaatttgtattttgtgattcacgcagcacctacatgattttaaattagttacacctactttatgaatatgagacatttattcagtctacagattacatttaatgtgcatatacacttgcataatttttgaagtaattagccaagatagtggtgttaggttgggaattaaggtactacttaaagctgtgtactttctcttagcttctgtttcttcatttaccatgtttgatctagatttccacttaataaaatgggaatagaaatatactcatttcagaaaattgttatgagggtttgattaatttgtaaaaagcacttagtacaatgtctggcatgtagtgtataataaataataattgttactctgtccttcctttataatagtacttgactactccataactgtagtaagtcctgataaacacatcttgaagttatccacatatctcagccacccttgatttcaacagccttttcataaaggtgatttctaaatctatcatcttagtgcagtcttcttttttttcccccttttttcttctatagccttcaaatgagaagtgctttcttctttattggacttgtaaccatttcaaattgaactaatgttaaactaaggttaactgtccactcccccatcagcttttccttctattctaattttctaatcctaatttctcttaaatagtaccaactttctttcaggtactcagacttggttcctagactgtcaagagtggaagggatcccatcagtcatttagtttagtagacacatggatgcttttaatccttctacaaatgtttttagtttctttaccatgtgttggcactttggacttgagaagacatagtttttgcccttaagcagcatgtagtttagttggagagacaggtaaatcagtacatttgtggtgagtgctgtgtggttttatgcacagattactatggtagtaactccagttgcaaatggatttagctaagaaaggactaaaggacattttagacaaagggaacagcagatgtgaagattcagaagcagatgtaatacgatgcctgtagggaatagtaataataatagctaatgtttattgagtacacactggcacatactgatgtctgaacctgaaaaatttctagcatagctggaatgaaaggtcaaggtgtatagtggggtggaggatgtggtagtaagtaagacaagacacctggggcctgaaatctaggtctttagcctcctggtccagtattatgccatcattcaaatttctttttgcaatttatatcagtttcaaattttgtgtaatgttgtgtactggttgaaatgattagaccattgcccctaggcctctgaccctaaagcctggactcttttaactatatttgctaaagtatatagttaggaggcattctgtaagcttttggacaaaaatataagagaggcatttcaaaccaggttgtcctgatctttatttttatggctcccctctgcctccctgctccctttctgagctttcaagatattaggttgaataaagagatcagcctttatggttgtcctacttttcttctgctccagattcttctttataaattgggagataaggaacttttattcattcaaccttcatattctgttaaactggtttactttaaccaaaatcaagaatgccactatataatagtttattgtaagaaaactaacttgtaaatagactcttgtcaatcacttcattttattttcatggagaagcctgcttaagaaattcaattaagattagaggccgggtgtggtggctcacgcccgtaatcccagcacttcgggaagctgaggcgggtggatcacctgaggtcaggagttcgagaccagcctggccaacatggtgaaaccccgtctctactaaaaatataaataattagccgggcgtggtggcaggcgcctataatcccagctactcgggaggctgaggcaggagaatcgcttgaacccaggaggcagaggttgcagtgagccaagattgcgccatcacactccagcctggaggacaagagcaagacttcgtcaaaaaaaaaaaaaaaaaaaaaaaaagatagaattgctatttgagaatttttaaaaaactgagtgattttaaagatcttttccattaagctttgatggaagtcagagtatatatatttctaggcaggccaaaatgaatattcatatagactagatattatagttattcttttggagaatactgggtatacatccatcaatacttattcttcagctatcatttttttttgtagatacctttgtttccacaaacctctttgttttcatttgttagtaatatcagcttgctaatgcccttgctacacatttccaatttctaaagttaaggctggcttcaacctgtcaacttgtaaaaagaccataaaaatcacctgttgatcaaactaagccaggtttattacatactccagtaagggtgaacactgccctaaagagtcctagaagtatttcagaaagaaagtattggatatggatatttacagggttttggggtctggtttcaagtagtttaaggagattttacttggccctgttctgagtttgtgatagaattcttagaccaagtcttgataaacaaatatttgtttggtaagtgagcatgtttgcccagttgagcaatgattaaggatctaattgagcagtctgttgttcagttaaatgaatttttgagaaattcctaaagctaatgatgaagttatttactggtttgcattcttaccttcctgggcaagaatttccgagaacaaataataaagttctgttgatataggggccttagatcttagtccttataattaagctgtgtgaatgcagaaggtctcagttctcaatgacatttataaaggcagacagattttaatcaaaggtttactgtctccagccctggatattgtaccatttcttgacctttttcatctaattgtggatggtagatagtatcatgattgtgagtctttgtggcacttcttgtacttccactaacatttaaatacctcatttatgttttgctactttcctaaatgcccccgaagctctagatgaactagtctctttttctcaacatggcacttttgcttttccctcttggttattttcctttatctcatctttgcaaaatttttattcttcatgcaggctttcctgagcctcatcatgtttatctttgaactagctatacctttttttctatgaagtcatttatttggctaatttagcactttattagattgtaagtccatggaagacagagactatgttttattcatcttatttttaatcatctaataaatatttgttaaactgaattactctgttgaattgagttaggccaaaaccaataaatacccaataggtttgtgaattattttcatattagactgatcttaaagaccttttatctttatcactatggttctctgtctttccaaaggtaaagtgtactctggaaaattctcattttagattgcagtttggacagatcatcaaagtcaaatgttactatgttttgaaatacagtcagcaaagttctcacagatggactttactgaaaacagatggcgggaggggggagatatttcaaaatgtctttgtgctcatttcaaagcattgacctctacaagtttaaatatagttattcatagaaaaacctggagaattaagttttatgcctttattcttggcagctttggttttgtttaatctgtcacaatgtcatgtttcttgtcataattttgttgagggtgtggtaaggagtatgacaacagaagcagaacatagcatgtatgtgatcattgaatatagatacaatttattgttcagagaggtatctttgggtactgtggtttggtggctgttggatggaaattagaaatagtaatttcactcataataaaatagttatttggacttaatattggaagacagagactattttaaatatagcgtctagcatagtctttccaagccatcagtatgtgacatatggtataccctagggaaagtatttttttatcctgttttagccagtgtttaactctgccactgtattcctactttatcctctctctaacccagggaactgcatttaccaccacccacaccacgtggataccgcattagcaactccgttaatacttgaacacaataaatagttaattatggtctagtattttaatttatcctttgagaacatatttgtgaatattcctttatacaagggtaggaattggtcatagtacttgataaatcaacagatttattaatactttgcccttctgaaggaaatatttttgtttttgtttgtttctcttgttgcccaggctggagtgcaatggcacgatctcggctcactgcaacccccacctcctgggttcaagcgattctcctgtctcaagcctgctgagtatctgggattacaggcgctcgccacctcacctggctgattcttgtatttgtagtagagacagggtttcaccacgttggccaggctggtctcgacctcctgacctcaggtggtgcacccacgttggcctcccaaagtgctgggattacagatgtgcgacactgtgcctggccggaaataattcttttataaagattctgaatcttgctgatatattgggatggatggaaatatttatctttactaaagtaatttttttttttttttgagacagggtgtagctctgttgcccaggctggaatgcaatggcaagatcacagctcattgcaacctcgacctcctggggctcaaggagtcctccccctcagcctcttgagtagctgggactgcagacaggcacgcaacaccactcctggctaatttttatattttttgtagagatggggttttaccatgttgcccaggctggtctcgagctcctgggctcaagccatcctcctgtcttggccgctatcagtagtcttatatgtaaccagttgcacttagcctgataatcagaattactgattgtgagtaatctcactgattacttactgagtcacattgattcggagtgcagtttcatcatgtttacttaatggtagtatacattagccatattgtgttttatgtacatatctgtagttttgaaatggctgacagtaatttgtatgagaataaagactctttgtttctgtttctttggagaatcaaatacatgttgaatatgtgcaaatatctgagctattttatataacataattttatattttaaaatcggtctgctataatcatttcaagctattgcccgttttcaggttttatataattttaatggaattttgtcattttaatttcgtgtttaattaggcacagccctcattaacttattgttgaaagtctcctagagcatttattaatttagccatcatgtaagtatgttccagaagccatggggaagaagacagacacttaaaaatctagtttaggaaatgatacgctaaaaatgtaaaattctattaaacgttaaataaaacagtattagtcagtaaaagagctgtaatcagatattactgtcaaatgagaggcacagacttaagtgctaaagaagtcagaagaagtagtcagaagaaacagaagtcagaagaagcaattgctgtggattagataggttatgcagtctctaatgaaaattggttcttggtggggtaggggatgaaaaactattaaatactactatggtttggaaccccccatagggccacagtacatagatagtatatctgtgttattaaaattcatggggatgaggaaagcaatctaaaatgtcccagggccgggtgcagtggctcatacctgtaatcccaacacactggaagctgaggcaggaggattgcttgaggccaggagttcaggaccagcctgagcaacacagacctcgtctctacacacacgcatgtgcacacaacagacacagtggtgcatacctgtagtgcacgcatgcacacacacgtgcgcacagtggtgtacacctgtagccccagctattcaggaggcttaggtggaggattgcttgaggccaggaggttgaggctgcagtgagccatgatcgcaccactgtactccaggagcctgggtgacagagcaagaccctggggtctcaaagaaaaacgctgtctcaaactaaaaaaaaaaaaaggtcccggggcttgtgggtagcagtaatgaaaggttgaggaacacgagtttaggatagttaaagaagggttttaaataggaactatgaaggttagtcattgaaggatagatggtaagttttaggtaaacaaagatggagaggttattttattcaagatggttttcttggagattatgtgtttttataaaatcagactgggtgtataacatgtctcacatgccgcatcatagcctctagttagtgtcttataaactgaagaataggaaaattaaaatctcagaatttccttttaaacaagatcaaatttaaagtgttgtagtagattacccattgcctctgtgttcattcttaccaagtagttttttgattagaggtagaggatttattcatttctttgtgcagtcatgttagcagtactgtttatctaaacagactcttggttacatacggaattatggttctcttgtgctgtcttattcattgagttaaagaattatagaatttttttagagatgtagaagactgtagagattgtttaaactcctccattgatacatagggagaaaatggacctgcagtgaagatccattgatgttagtggtggagctggcctcaccactttggtctcctgacttgcagtctactgcccttcttctcacaatatcattttatctaaaccagtatgaaccttggtctggaggcagaaattttaattttagtaatttatctctttagaccataccagattgtcacttctttttccatttctctagacaaatcagtcattttctccagaatgtggaaaggcagtgggttttggagtttgacttttccacttcagctttaaccttggacaacttacttaacttctttgcctaatgctcttatatgttaaatgggaatgagaatacttacttaataaggtggttgttaggattaagttagatgacctatatataaagccttttcctctgtgttggatatatttaaaaaatttattaaatgtttttctcccctttctttctctgccactagagtacttaccacattgtgtatcacagttagttattttatgtgtcccaggtgcgagtcttggaggttttccccttagtaatttctcaactttatgtaatattattatggggtctatgacataatttttaaaaaacaaaccagtaaagccaggaatttgctactgaaacagagatgctgctatagtagacagttaattcatctgtcagagaagaattgagtactagtgttcatttttaatactaataattctataataaactgacttactatattgttgattcttgtctaaggccttgttttgaccttaagaatttcattataattccacaagggaaatgtattgattgtgtcatttggctcccagtctctgtccttgactgtttctgtgattgccttgcagagctctgaggaacatgaatacagtgatgaagctcctcaggaagatgagggctttatgggcatgtcccctctcttacaagcccatcatgctatggaaaaaatggaagaatttgtttgtaaggtaagagtgcagtttcttgtcattgtcatgctatatatttagcccttccaaaacagaaacctttaaagagagtggtagtactatagcctttggtttgctttggggaagatctgtggcaagatggggacagaagtggtcttgggactctaatcctgttgaagacagaatacatttgttggcttgtagtagccatgataaagctctgataaaactaggatatccagctgggaaacaaacatttcctaatgtaggttaacagagtaaatgtcttattggtggtttagacaaacagaatgaaacatagtcagaagtggttgacgcctggagatatgaaaatgccctccgcaggtcccttaaatctgttctgctgagtgactgcaatggcaaatgtactgttctttctagggattctgtgagtaatcaggctactcttgagagagattttccagtttccatgtacatctggtattttcagttcagctggtatacattctgtctcttaaggtttttcttccagtccattgtcttgattccctaggacctattatgggattctttttttttttttttttttctaaaatagtaattagaattgagtttgcctgcttagagaaaatagacacactggtaaacttgtaggtacttgatcaatcagatcaataagggccctggcaggtagaatctaattaatgtaagtcgatcagtctgtgtgtatacgtatgtgtatgtaaattgatttattaaacatttctggagaactctttattatttagagagaaatttttgtgacacttagcttttcatggcttttatgaaaccaaaactttttatgtacatggtcttagatccaggagactaaatataggttttcttacattcacaataatgctggaaataattgggatcagtggacttagaatgctattactttggaccagggtatgaaaagagaacattgtgatctgaacatagacatcctttccaagactaagggtccagtgggttttcagggggagtttaaacatttatgggtctgtaatagaaaattttagccagtttgaaaatgagtgaagaaacaactgaacagaacaatagattcattctagtgatcactgtctcaggagaattagcacaaagggatacaaaagattattttgtttttaaaatgcaaatttggtgtgaagaaatctctcaaagttggcaaatttagtatctgggccattaggggattttaatatttagagatcagcggggtacggtagctgacttaactgcctttttgtcctctccctttctgaacagagaacaaagaataaaacaaataaaactctaaatttaacagcaaatcagagaataaatagggcaggacatttgtttgctacaaactatgatcttctgaaggaaaaggagtgttgtaaatatcccttagcagtttgttgatgacagcagaatgaagaggcatcctgttacacagtgttgtctggtgtggctcaaccctgccatggcagtgcaactgaccacacttgaaaaccagccgtgtctagtggcacagggattctatttcttggactggaagcaagtaaacagagacagtttaatatgaaatgtgctgtggataatttagtctttagagtttccacgtttgatcttggtcactcttttttcccctagacttcagtgtttgctatttaccagcataaccaggtccatgtgggaataggatgtatgctatggggagtttatttgtttttgaaatttatttattgttgagtctcgctctgtcgctaggctggagtgcagtggcacgatcttggctcactgcaacctccaactcccgggttcaagtgattctcctgcctcagcctcccaagtagctgggattacaggcatgtgccaccatgcccggctaatttttgtatttttagtagagatggggtttcaccatgttggccaggatggtctccatctcctgacctcgtgatctgcccgcctcggcctcccatagtgctgggattacaggcatgtgccaccatgcccggctaatttttgtatttttagtagagatggggtttcaccatgttggccaggatggtctccatctcctgacctcgtgatctgcccgcctcggcctcccatagtgctgggattacaggcgtgagccactgcgtccagctgtttttttaagaaatgtttttgtgcattttatggtctaatatatccacatacccagtggattattactaagatatttgaaggtaggcagtgtctttgtcaagcttataaataaatatgtctttggctaagccgtgatcttatttttttattctagggggtaaaagaggatacctatttgtgttggaatttggctgactgtatatgactccaggttgagaatgagggtgcagtgtctgttgaatgtgtcactaattgcagtgtaattaagaaagtatagctccttgtctcagtatttagaccactagaacatttgtttagaaataccagggaaaatcactctgaaattatatgcatatataaccaataagaaactcacatattctttttgaatgtaaaaaaggaacatatgactcttttgtctgtgtggggtagcagagtagcagtttggatacagcagagctgtgtgcacattgtgaattgccttgaagatgtaagggttgatttctgtcttcctctccctctcagaaaataaaattcccccaaagtaaggtaatagcaacattctgtaaggttaatctcaacaatagtggtcttttcccaaattcaaatttgatgcaaaaaattcagtatcttggtctctctctgacactaaaagaagcagactgcagaggttgacagacttccagcagttccaacagaagggatactggatccctgatcccctttctctttctctccataaagtcctgtggaagatctcatacagctacttttgtaagtggtagtactattagcagcagatgttaaaaatatagattagtcttttggcattattttattttattgtatgttagaagcaagcatagaatggccacattagcaccagtaaccacagaccccacgttttgagttggaaagtgagggaggagaggttttgtatggctgggggcaggaagagtagggaagaggcagggggctagtttggttagagtgttaagagggacacatctgttcttaaattgttagtcctcacttcctttggatgtggagatgttacacagaggattagttcttttacaaatatctgtaaggaagtaagagagcagagaacaagaaacaggttgtggagcaaaaggaatgggcacagtagctggtgagtaagtggcagtctttccatagagtagagagagagccaagtttccatggcttacccatattgtcacaagaccctgtagtgggtagggtggaggtcattgtctgaaggtgaatcttgctgatttgcagcctggattcttgctttatagggtaatagactggaagaactgctgagcccacattagctggaagataaaccatgtgcctgccatttttccttacatctttctgtctagcagaagcaagttagtcaagattaagtgatccttgtggtgtgagctctcggggctatgagacattaaaagaagcagagtgcagaggttgacagacttcctacagaggggatatcggattcctgatcccttttcctctagaatctgatgcgggagggaccatgaggaaacgggaggaaagggctctgagggactgaaagggtggttagggctgagcttctttaccatctctctgcagcatgagaaggaagatggattagtcactaactgtctgacaggagatagcccctggggatagtcatgtactggctttgagtagatgccagggattactaactaattcatgaaatgctttgtgtcattttgtttgatcttcatgatttccctatgaagtaactgaagcgctattttacaaatgaaggagctgcaggttggaggttttttaattaattaaatctcatatagctagttaatgccaggggtccacaattttctgactcctgtgctctttcttttctgcctcatcatatttgcatagtgggaagagaagaaagagtaagatggagggagagattttagcaaaaggcacaaaagtatgattctatgtaattctgtttttaccaataggaatattaaatataaaaatgttcatattaacaccacagggatttatcagaggtaaagtctgttatataggtaggatgaaggaggtcagtatgagtgagactttggaaccttcatcccaccattagccaggttgaaacaaagtttgaaaccactcttttagggcatttcctataaatttgtttactctttccttttcctaaggcccccacttaaaagtatattttttctttctttttttttttttttttttttgaggcagagttttgctcttgttgcccaggctggagtgcaatggcgtcatctcagctcaccacaacctccgcctcccgggttcaagcaattctcctgcctcagcctcccgagtaggtgggattacaggcatgtgccaccacacccggctaattttttgtatttttagtagagatggggttcgagctcctgacctcaggtgatccacccaccttggcctcccagagtgctgggattaaagccttagccaccatgcccggcaaaaatatatttttcatcagtctttaaagaactcagaatataagacattatacattttgagatgtttgcctttttaaaaatgaattgtttttcccattataaaacagtgtgataagacacagttgttaggggtcagtgttggtaaattgacaactaaaatcacaaagatgtttgataatacctctctgccaggtatgggaaggtcggtggcgagtgatccctcatgatgtactaccagactggctcaaggataatgacttcctcttgcatggacaccggcctcctatgccttctttccgggcctgttttaagagcattttcagaatacacacagaaacaggcaacatttggacacatctcttaggtatgtaatgtcagtgatgtaatgagctggtgattcactttcttcctttttattttcatgtatttgagggtaagcacagaacttcagaaatgtatttggatttgccattttgttttctgaatttctaatgatgaattttctgactggtttactcgtagtttatcctggtttgcactatgattttgttataacttaagttatatttctttctactagattttcagatcttgtcctcaaaccaggccaagtaaaagttgctatacaaaatgtggtttcatattgtagtgaccactgagaagaaagtattatttaatagaagtaatctgatcttttaaaatgcattacttatatgcactgctaattttcagtttggcctgaagtagaatatggactgtaattttttctccataatgattgtatattaaagaaagaatactgtagaggaaagaatagtctttccaacaaatggtgctgggataactgagccatatgtaaaagaatgatcccgaactcacatatgcaaaagtgaacccaaaatggatcaaagacccaaacataagagcaaaaactataaaatccttaggagaaaacatatataaatccttgtaagtttggattaggtgactgtttctgagaattgccaccaaagaaaaaatagataaattggacttcatcaaaattaaaaacgttggtatatcaaagtgcattatcaagaaagtgaaaagacaacccatagagaagagaatatttacaaatcagctagttggtaaggatctaataaataactcctacaactcaacaataaagacaaccgattttaaaaatgggtaaaacatctgaatagatgttcttccagagaagatctaaaatggccaataagcacatgaaaagatgtttgtcttcgttaggcatttaggaaaacgcaaatcaaatgctttgagaaatgcagtgagaaatctcaacacttcacacccactaagatggctataatcaagaaaatgaaaaataacaagtcttggcaaggatgtggggaaattggaactttcatacgttgccagtaggaatgtgaaatggggcagtccccatggaaaagtctggcagttcctcaaaaagttacatagagttaccagaaatcaactcctagtatattacccaagatgactgaaaacatgttcacataaaaacatgaacacagaagtttgcagcaacattattcataatagctaaaaagtagaagcaacccagatgtctatcgactggtaatggagaaacaaaatatggtgtattcatacactggagtattatttagctgcaaaaagggatgaagcactgatacatgccacagcatgggtgagccttgaaaacatttgctaagtgaaagaagccagacacagaaggtcacatgtcgtttgattctacatgcatgaaatgtccagaacaggaaaatccataaagacagtagattagtggttgtttagggctgtaaggagtggggaatgggggatgacagcagatacagtacttctgtttggggtgatgaaaatattttggaattagttagtggtgatggttgtacaaccttgtgaatatactagtttctggccttactccatttgatttgcatttcttatcacacatttgatttccatggtgcctctaagttcctgcctgctagtatagtgagaggagagtagagggtcaaatcaagtttgttgctgacttctaattcaagggtaatttttaattaaaatgataaaacaattacttagcaaacgttgaagttgaaatttctgtgcctgccttgtgttaggtggcccctccctaagcaataggaacatggcatctttctgtgtagaacagtgtaagaattggttaggaaatggcctgagtaggcattttgtccttaactcttgaatctcctgaaccagctgatgagggccagagaaacagcatgacactccctgacagctattggtagaggaaagctgtatttcttggcagggtttaggggtattggaattcattgtaaaaacaaaagcaaaagcacaatggacctttgaaagctggactatgaggtatgtgccaaaaactgtataaatgaaactattctacatctagaggtagaagtggcttcattatatcttctaagaagtagcagcagtgtctgatttctttttagattatgcccttagataagtttttatattctgtatattcagtttatgaagctgaagtatttctgataccaacaatgttggaagtaaaccacaattttggataggcttcagttatttggaagatttttctcattgaacttttgattcagatattgttgtaatgcagaaggattgaaaacagaatcagttataatactgtctcttctaaagggtgatattttatctattttctgtgctctttttcctaggttgtgtattcttcctgtgcctggggatcttttatatgtttcgcccaaatatctcctttgtggcccctctgcaagagaaggtggtctttggattatttttcttaggagccattctctgcctttctttttcatggctcttccacacagtctactgccactcagagggggtctctcggctcttctctaagtaagtatctgtaaagtccgtattttggccaatgatttagaggtagtgcgttagggaaaaacattcagcagagttggcagaattcttaatatcatctcatgcaaacttttttttttttaaaaattaaagagcaacccagtttggctcttaggtgtcgttgatggcttatgtcatgagggattaattggaacctgtagtggccagtagtttttaaattactgcattaaatggattttttttttctgtcatagactggattactctggtattgctcttctgattatgggaagttttgttccttggctttattattctttctactgtaatccacaaccttgcttcatctacttgattgtcatctgtgtgctgggcattgcagccattatagtctcccagtgggacatgtttgccacccctcagtatcggggagtaagagcaggtaagagcacggggaggttctacattcgacattcatttattcactagttaaattcactatttattaaagttctaccatttgccaaacattatgctgtgtatttgtgatgccaagatgaagaaatcatagttcctattgttgggggtttttttatgtagtagtcgtagtagttgttgttgttagtagtgtttattaattgtcttaaagcattagggttgaggggtcatggtgagtatgaggtgatataatttgggactgttacttacttgaatttatggctaaacaccatccagaaagcggtagtctataacttttgtaaacaatgatttctcttactcattgtggagtgtactttttgtagtgattctttttttgttgtcattttctgttttgttttatatttttattttataatggataatctgcttaaggcttcacccccatgctgattatgttttgttaaagtgggcaccatatctcccttctgatgtagggataaaggggctttttatatttaggataatgcctgtttagctggagatcatagagaaagccacaagcccctcagtgccttggctgagtcttcttgtaggacatggaattagcagagatgttcttgtctcctactcagtgtcaagctcagtgcccactgagaatgaacaaggagtgtttttcctttcgtccttaaacccccaaaaccatttagtattaaaatctaaaccctggatcatccgggggtacctggaaggttagacacagaagtaacctcatgtcagtgaccactgttaaaacactctgccgtcctcctttttgtgttcatatgagcaaatgagcattggtcagggtcagctttcaacagtacttgagtagtacagtggtgttaagcctgcttgagcacatgagataagaatgtctttgctgagtacagctgagcattctgttatttacatacatcagaagaaccaaattcctgagcctttgacttagagaatgataggcagaaaccattagcagcctttgcttagcatgtagaggaaaagaaaaagtgaatagtgcaaaagtctagcaaatctttccttagaggagcagaggtaggaaatagtttttctttaggtctgcttggttcttacctgttgtgagagcagcattgccattttcctcttctcttttagatgtccattttatttgtgagtatgcatcccatagctgtgcttacatatattgctgtataccagcagtttatttggtaatgtccaaagcaagtaataggttctttatctgttgagaaagcttaccaaatgacagcttgttttgcaagggtcattgttaactctgtgtcaaaatgtaggagcagttactggcagaggcagggagctcctgtgttgctttggctgatacacgggtttcaaagtggatttagatcagtggacttcacatttttttttccatcatgacattttctattacagtcctgaacacgtgtatctatacataagttcatgtatatataaattacaaaactttaacaaaatagtatttaatcctccacgtatgttacagtctgatattttaaattttattctagttcatttaaaaaaatgctggttgagactcatttaattgatcttagaacttaggtcatatccagaagtttaaaaaaacattagatgcttctggaggcagtaaggcagtagggctgtggctgagtgatttgctagacttttgtacttgattgctatttaggggtctgggagcttatagtattctctcccatcccccacccccattaggtcatttgttaaaggcatttaacaatttttaagtgcccagttataggttatagatttagattaaattttttcttcttttttcttttcttttctttttctttctttctttcttttttttttttttttttttttttttgagatggagtcttgctttgtagcccaggctggagtgcagtggtgccatcttggctcctgacctcaagtgatctgcctgcctcggcctcccaaagtgctgggattacaggtgtgagctgagccactgcgcccagccttagattcctattaaaaataggtgatgccaggtgatgtgatcattcatcgcctctccaccccaacaacgcctgctgcttcaaagagaagatgggatacgtaaaattgcttgtattgtaacctgtgtctgtagaaggacaggctaggacagtttaaataaactcctggagctgtcctttccgaaaacatttagcaacattaattcaaaacccttgaagactgctgacttactaagctaagcctctcttagtgaggctcattgctttttttttttttttttgagacggagtttcactcctgttgcccaagctggggtgcaatggcgtgatctcggctcactgcagcctctgcccccggcttcaggtgattctcctgcctcagcctcccgagtagctgggattacaggagtgcgctaccacgcctggctactttttggatgtttagtagaaatggggtttcaccctgttagccaggctggtcccaaagtgctgggattacaggtgtgagccactgcacccggctggctccttgcatttgtaaatctttagaacatagctgtgttgtcttgtgtgacagtgtcccagagtttgaagaaccttctttttcctttaaaactggaagctaattttatatgctactttgaaatattcagtttacagagttaatggtgctgtgctgtaatactgacttctggctctttgtttctcttctgcattactttactctcttcttgtgactcctaggagtgtttttgggcctaggcctgagtggaatcattcctaccttgcactatgtcatctcggaggggttccttaaggccgccaccatagggcagataggctggttgatgctgatggccagcctctacatcacaggagctgccctgtatgctgcccggatccccgaacgctttttccctggcaaatgtgacatctgggtaagtatgtcggggtgactgagtgtgtaggtatctgctcatgagttattggcatcctgcagagtgatgcaatagaaaaagttttagacacaaagtcaggagagttgtatcctggtcctaactccctattgagctttctctggacaataagtacaataataactaacatttagtgagcacttccttggtgctcagcatggtacagagtgctttgcacggattacctcattacatacattagccctatgtggtatgataactcttgttaatgtcttcattttacaaagcaggagactaaggcttagggaggtgaggccacttgtccaagccatgtggctatttaagtggtagaacaggaatttgaattaggctagttttaatatcagaatctgtgctctttttgacaatggcaagtcattttgccattctttgtctcagtttccttatttatgaaatgaggatgatagaccagattttcaccagagccctttttaggaccagatttctatcagggtctttttttataccgaaatttagtgatttgtaatgggagactgagtgtatgtatgtctggtggaagaagagggattgtgaagaagagggaactggattgaactggattgtggtcattggtaagagaggaggaaagagaagagggaaaagaaaatataaggactgatatagatagaaaaacttataaccaaagagggcctgaaatattttcaacaagacaacattggaaatacgcttaatgtgaaatctgacctttgtaattctgttcctcagcctattgctgattattatagactaacctgatgatctcatctctttaaagtttagtttagctatctgtaaaatgaagatgatcttgaacttaagcaatctttctagcttcaaattagctaatttatctttctgattcatcatcatcattttaacattggtatggaggggtttttgccctttatcccctttctttgggggaaattaatcccctttggtttcctaggaaacatctcttgggatgtgacttgatttcttctagtttttgcttctaaaatcagtgtttttctcttgataaatactgctttgccactgtactttttcattttgagattataatgacagaatagtgtaatttttgaagtgcagcgagcaattgattacccttcttgagatgtggaatagcattcaacagaacaggtaaaatcagaaataatgtcacttgcttgtagaccagcttctgacttgagaagtaaaagggcctgtttgcttagtttttgacacattatgtagttcatgaaatttgagaatcgtagcactgaaggaaactctggggaccatatgttgtgacattccacacaccatcctggaagcctctttagtttttctaacagagagcaacatttaaatgaccatagctctagtcctcttgtccatgacaaaatgtccatcagcagtgagtaggaaaaggactgtatgtgtaatcatagcccatggctaggtgtcaagggatccttgggacctttgtggcctgttagcaagagagtctgtgtgtcatttgccactttagcaagagagtctgtgtgtcatttgccactggtttcagtgttaagtttgtagatttttatgtgtagagcttagtggcagcaccttggtttctttgggaacagctggctttcagctcagcagaatgtgttacacctaatggatgttccaggatctgtggacgccttgaatttgagctctgtcacatccattccctgccttctgctcagcataaaccaggattaaacgatcctaagaatctttagcagtcttaataatgtatgggtttctctacccccttctcttcttttttcccctccagtttcactctcatcagctgtttcatatctttgtggttgctggagcttttgttcacttccatggtgtctcaaacctccaggagtttcgtttcatgatcggcgggggctgcagtgaagaggatgcactgtgatacctaccagtctccagggactatgaccctaaaccagggcctgcggcacttgcgggcctccctgctggctactgatgccagtaccagaggagccccaaaactttgacagcctcgtgggctttgtgacggcccagtggctctgcgtggtacatgactgagaagagaaaaacaaaaataaatcatacctcaaaggatggagtgcatcaattgggagaaaaggagacatagcccaaaccctggcttattcttgggatctactgattgcgggctctgcaagacccttggcaaactggcttctgatccatatcatatttatttgtagaagatggcgaaacagtttagctggtggttctttcttctccctttctctctctctatgacaataatacaaaccaatttaagtgaacatttatatccgataaggggtgggagtgtgattttaaatgctcttttgggagaacaaagaaattaatgtaaataagatttctaactgtttaaataagactttatataaatgtttaaaacataggggtaagggagggagggagaatttttgtatagaatgaaacatgcaagtaccacacactgtttgaattttgcacaaaaagtgactgtaggatcaggtgatagccccggaatgtacagtgtcttggtgcaccaagatgccttctaaaggctgacataccttggaccctaatggggcagagagtatagccctagcccagtggtgacatgaccactccctttgggaggcctgaggtagaggggagtggtatgtgttttctcagtggaagcagcacatgagtgggtgacaggatgttagataaaggctctagttagggtgtcattgtcatttgagagactgacacactcctagcagctggtaaaggggtgctggaggccatggaggagctctagaaacattagcatgggctgatctgattacttcctggcatcccgctcacttttatgggaagtcttattagagggatgggacagttttccatatccttgctgtggagctctggaacactctctaaatttccctctattaaaaatcactgccctaactacacttcctccttgagggaatagaaatggacctttctctgacatagttcttggcatgggagccagccacaaatgagattctgacgtgtccaggtttctcctgagctcatctacatagattggtagacccttcctttggattaggaaagatgagttttacctctggtacactgtcttggtaagcctggatgtgacagacacctcggctctccttgaataagaaagccagcagaactcttaaagccagttgtagtacggagttgtcagcactcactgaacctcactttacagggataagagtggtgtggcattttaaatacaatggtatgttattgccagggagtgaggtacaagacgatggctcatgtcacaggcctacctgatacggtgtcagagaaagtggtggggaaaggatctggttcatggaattctgatcttggcccataggtgaaccaccaaaatagtgctcgagtcttaggttactgtcatcaaagacttgggatgactccattatatcctggggttgtgggtattagaactaaatatggaggtcctgagcatggggactggcgtcctcagtaggtgtttgggaatatgggaagggtctcctatttattcaatagagttttctcagttattttcctcccttgcccttgcaatctccagcaaaaggtgggatctaggaagaaagaatccagtgtagaagttgagaagaacttgaacgttttggttctggataaggtcactgtcctaggtgctaggtggaccgagcaaaagactcagtggatgaactggtgcagtgcctgacagaataaagaacagtattaatccctttgagaaagcatagtccagcaggacagtggccatttggacagaagcccacttagtttcttgggagcaacagcacgtatcagaagccagacttgctcttcggtcatgcactttgggatacagcgtataggtgcagccctgtcacaacaccaacagaagtagcagcctctgggtgcagtcacccacaccccaaagctggaaggatctggttcaacatagcacaaacccttaggaaaaatgaaattaacatcactgatgtgtaatccagtaaaatctccctttttcgggtgtgtatgtgggcatgtgcccatttctatgtgtgtgtctacgtgcagctcactaccaacagcctcatgtgcacttgacctgacagtgctcgctgagaactctcaccaggttggcgcctgaatgccttactctcagcagtcagaggcttgcttgctctgtgcagatttttaattttcttttttggccctaggctggttgggacctctacagcttcattctttcaccattaaatagtggcctttttcagtattttccctcttcccctttataaattatgctaaagccacaaagcacatttttggggatcatagaaggttggggttccagaaaggcatctgtgtgatggttccattgatgtgggatttccctacttgctgtattctcagtttctaataaaaagaaccaaatgaaatatgacctgtcgtgtgtttttttattgccgttcagcttacctccccttggcttttggcacttctctgaagctctaatttagacactgtgatttaagaaatttttg

SEQ ID NO: 11 - DNA, 16994 bp, Homo sapiens
tggtactggccaagttttctctaaaggataaagcccgaactcagcccctcagacagtgaataagaagtttctttttttctttctttctttcttttttttttttctcgagacggagtctcgctctgtcgcccaggctggagtgcagtggtgagatctcggctcactgcaagctccgcctcccgggttcacaccattctcctgcctcagcctcccaagcagctgagactacaggtgcccaccaccacgcccggctaatttttggtatttttttagtagagacggggtttccctgtgttagccaggatggtctcgatctcctgaccccctgatctgcccacctcggcttcccaaagtgctgggattacaggcgtgagccaccacgcccggccaagaagtttcttttctgtatttttacttttttttattttttgagacggagtctcgctctgtcacccaggctagagtgcagtggcccaatctcggctcactgcaacctccgcctcatgggttcacgccattctcctgcctcagcctcccaagtagctgggactacaggcgcccaccatcacgcctggctaattttttgtatttttagtagagacggggtttcaccgtgttagccaggatggcctcgatctcctgacctcgtgatccgcccacctcggcctcccaaagtgctgggattacaggcgtgagccacggcgcctggcccaagaagtttctttaaggcagctccagctttttcagtcaaagtgaaatggtcctgaaatttgcatatgcaatgtttacctttctttttcattaaacgagagagggaaccaaaacaaaaatacagtttgaccatttaaaatgttggaagagctgccaacttggtagcactatctagagtggctccattctgccatctagtggaagggaaaagacaaatttagaccaaaatacttccatctttaatttacaaaaaagtgtgttcttttgttttttgttttttaaagttggggtcttgctctgttacctaggctggagtgcaagcagtgtgatcatagctcaacagcctggagctcctgggctgaagccatcctcccacttcaaccttcctggtcactgggattacaggcatgagccgcagcaccaggctgtgttttctgattagcataatttaaattatctactaaatttgctcgttaatagattcactttgtaaagaaaaaccacacagtattctaaacactaagcagtaaaactaaatttcccaaaacatttatatttataaactatgaatttcaaagatctgcagaggaaagagagaacgttcttgagctcttattatcataaattttcctgcccctccctttcccgtttatattatccatttctacacatactctttacgtttattttggcacatagactgggagaagcccagagtctctgaggattatatctttcccaaaatactgagcaaaatcagtgggtgagtaagatcattccattcatttttcaaaaatgtacactcacttaaaatgttgatgtatccgggcatggtggctcaagcctgtaatcccagcactttcagaggctgaggcaggcggatcacaaggtcaagagattgagactatcctggtcaacatggtaaaaccccgtctccactaaaaatacaaaaattagctgggcatggtggtgtgtgcctgtagtcccagctacttgggaggctgaggcaagagaatcacttgaacctgggaggcagaggttgctgtgagctgagatcatgccactgcactccagcctgttgacacagtgagacttcgtctcaaaaacaaacaaacaaaaaagttgatgtaaccaaggtatttgttaaggcctgtgtgcttgaatttgttttttgctggtgtatgtatgcatgtttgctttgttttgatttctatggacattatctaggtatttatgatgatttccattgcctaggatgaagacatctgcagccaaaactccctgaaaagttagtaacagtagcatacctgaagtgactggtgctattctgaggctgttactctttttttgaaacagggtcttgctctgtcgcccaggctgagcgcgatgacatgatttcggctcactgcagccttgacctcccagactcaagcaatcctcctgcctcggcctcccaatgtgctgggattacagcacaggccaccacacccggctaatttttaaagttttttttagagacggggtctccctatgttgcccaggctggtctcaaactcctgggctcaagagatcctcttgccttgacctcccaaagtgttgggattacaggtgtgagccaacacatctggctggttattactcgtattactagcaaatgttatttattgagtttctatggtgtactagacactgttctttttttttttttttgagatggagtctcgctctatcccccaggctggagtgcaatggcacaatctcgacacactgcaacctctacctcccaggtccaagcgattctcctgtctcagcctcccaagtagctaggtttataggtgcccgccaccatgccaagctaatttttgtatttttttttagtgcagacagggtttcaccatgttggccaggctggtctcgaactcctaacctcaggtgatccacccatctcagcctcccaaagtgctgggattacaagcgtgagacaccctgccttggccagacactgttcttgatgtacattttgctaatccttccactaaatctccaaagtagatatcattgtcttcactttagaaataaaaaaatgatgcttatgaccacctaccagcaaatcttgatgcgccagggactggggggaataatcattgactagctggctattatgagtcacagtttccattcctgcaaaatgagaatttttttgacttaggggtggggtgttctctaagctttttgcctgattctaattcttctgctagaagctgtgggtttacagaggtgaaaaagatagcccttccaccaaacatatattctattatatatatcttatctatatatattatatatattctattatatgtatcttatctatacatattatatattctattatatatatcttatctctatatattatatatattctatttatcttatctctatatattatatatattctattatatatcccttccaccaaaaatatattcttttatattaataaaatataaacccagggtccaatggaaaagacttaagggcagacacagagtgggatgtggtttgtgttaggagagggctgaagggacatcatggcaggcatttgtgtgacctcccacaaaacaccctcctccactctgacctcactttgttcttccttccctttgtccttattatttcctttctgcttctctttcttttctgtctccctctgccctgccctagaacggtgcctttctaattgtgggttataacacattattagtaggtcataaaatcaattcagtggatgtggaccagcattttaaaaagggaattaatagaatataaactccttaaaaggcgtcactcatactcaggataatactgctgtgtaaaatttgttttaatatgtgtcagcatgtgtacactgtgtcaagatgtaacacttacatcctattttaagaccctgctgtagactaaaccaggacccccatggtgctccaggcgtccccaccctcacctgtggcaccaggagctcctgctgtcactggcccagctgggtcctgtgtctttctctgcgttgtcagggcctgcgctggcagtccacctacagggaggtggaatccggggcttgacggcactgggaggatccttcatctttccaggctcaagcaaatcctcgcctggaggcggggaatgggctagatgacccctgaaacagtccagtccagcatctgagagctcctctgggtccagccctgtgccgcctttccacgcttccctgcagggccctggggagagcagagaaagcagctgccagctccaggcagtcattttaccccaggtgccacttaccctgcggaggggctggaaccagctaatcattttaccccaggtggcacttaccctgcggagtggctggagcttctcaggtttttcagttcctgtgtcacctcccagccctgcccaccacatgctgtgggtgtctatggctgacgaacgcccctgagccaggccaacccccagcctcccagtgggtggctctgtggtccctggcagccccggcatcatccggagcacagaggccatggacactcaggggatggtcaaggtcgtagctccctccgttcttccgttcctccctcacagagctttcagcaagatagatggtcattcttctgtcctcactgctcccggcataaggggaccccagggctggatgacttccctaaagaggaatctctcagggtttcctgcagccgctcccaagtcccgtctggaggatgaaggtggcggacacatccctgacgcagtttctcctctgccctccacccctcacccatcccacctcctggactaggaactcaaccaggacttgcttcaacctgagaagatggtgtccagcccccttggagcctgccagcatccggcgggatttcagaggggcctccagggaggcgctgcccagctgcccccagtgttgctcacacagcccccactgatctgctctggggaggccacggccccagaacgcggcttgtggggggcctggggcagcaggccgggagacgtcacttgtccgctgcctgctcacccagataactgcagctccctccactgccgcctacagccctcccgccatgcctctgcccagaaggccctccctctccagtcctgtccgcccctcaaggcctgcccaaacctccctgaattctcgcctgagcagacactttcctagcacggctcccgccgcgtggattactcagccgagcctcagtctggggctgccctgtgctgggagctttatgcagatgctcggctccctaccctggcttgtgagccgtgtggcgcagaaaccgcatctcactcagagcgagggtaaagagcagcagggctggagaagccagctgccctgccacaggccaagaggacttggccaagtgaccattgctgtctgtttccccaactgtaaagggggctgagacccttacagggttgtgaggcttaaactttgacaaacgcctaccatcccaccaggcacagggcagttgctgagtatttggcggtggctgctgttccccacagtgtccaacacctcccggggatgtaacaggtgcaccacgggcaagactgatgggtttatcagttcactgcggagagaggcagggatggatgcacttgcttgttcctcttattttcccttatggggagcagtgacacttggcagaggctgccagggagatgggaaatggacattaattacgggaaagaaaaggaggcaagaaggggaggagggtgggaaatagacaggggaggaagaagggacggaggcatggttagaagacaggcaagcaaaaaagacgactgttttcttggacgaacagggaggtagggccatgaaaagcatgtggacgcttcttctcagtgaattaaggcatttcagcgctggaaagagcctgaaaaaatcagactcccttcacagcccagcggagtgcaggccctgggagagcaggagttagagatggggaggaccccgcctgcaacaggcactgcctgctacaccctgccatgggcagaggggctgtcctcccaccccagagaggccccatcctttcctttctttccccaaggctgctccagcagggagtttagctccatggacacctttgaaggctatgaaggtgcagaagacatggaaaaggtgagcagcgctctgaacgtaatttctttctttcttgtttatttcatcttcttcctccttcctattcatcagctcctcctgcctactgtccctcgcctccatccctctccccctcttcaggattctcacccataaggacctggttaagccagggaaccgaggtcactagtcctcaaagtggctctgaccctgctagcccggcatgtccccacgtggctgtctccttggcggtgcgctggtgtccggtcataactggggctcccgctgagagccccgtggcccaggtgggagggctggggtgggcctcagcctgccagcggaagtcaccacattgcccggcctggccacagcctccagcaataactctgagggtgcacccgtccccccccccgcccccccttccagctcgcggggccttttccagttggatgacagtcacgaaacctgttcctccaaatccgctttcctggagaggagagaaagcacccagggagaaaaagaagaacgcgatgaggaagcaacgtgagtgtgagcgaggaaaactaagcgcctctgtgggccacggcgtctctgactttcttctcggaattgcagggcgaccggcagcaccaagaaggagcgtgctggggcgtccacgccggctgactcctgcgggggcgacggctggtttccaggcgagggcgcggcgcccgaggacctccaccccggagagctgccctccctgcggtcgccccggctccccggctccagagcgcccgcattcccgagagaggcgatgtgggggcccgggccctgcccagctacgcagaaagcagccggggccgcggcggcggcagaaaggggacgcccagggccgcctcccgggagcccgaggggtgccctgcgtgctcgtctagctcccgccgcccggcgaaggacctcgcgggacggggacccctagtgcgcctgcgcgagcgcggcgggcggaggcagacgctcgcggaggccacgcctcctaaggctcggccgagcctaggaggagccggagctggctcggggctgctcccacccgccagcgccggctgcccagcctggcagtggaccgggtgtgcaagccacttagcacctgacaggttaattcagagggggtggttccaaggtaactgctgagagatttacgtttggatttgttagtttaaaaattagttttccagttacccgggctggagtgcgatggcgcaatctctgctcactgcatcctccgcctcacaggttcctgcaattctcctgcctcagcctcccgagaagctgggattacaggcatgtggcaccacgcccggctgatttttgtatttttagtatagatgggggtttcaccatcttggtcaggctggtatcgaactcctgacctcaggtaatccgcccgcctcggcctcccaaagtgctgggattacaggtgtgagccactgcggcctgccgattttcagaattttcgtcagaggtttgcatctgtttagttttttagttatcttcagacttgtcatgttttcttttcataagttgcaaattaacttttctgtttttaatttttacttacttagttttaaggtgtatttactttcagaatttcttaggtttaacattgctgtcagtcagggtccaatcactgaaatagaccccacaccaattattttaacagaaaatttaatataaggaattgtcacacaactattggagaactaaaaaggcaaaaaaaaaaaaaaaaaggatgatattgagatatcatggagatttcaactgcaagaagcagctactacctcaagagctaagggaacaaaaagaagttggaatgatcaaaactcagaagtctgaagatggccccagggctggggttgttctttgcggaggagacactgctcagagcctggggtctgggagctggatgcagaactcgggaggggacatggccagctcgtgctggtgtctctgaatgggcatcatcaggctggttatgggagtgctgggaaaatgacaagctggaaccaactgctgctactgacaccacctgtcattctcagagtggagacctgttgctagaacgctgacaggaacccaggagcacaggaaggagcccatcgtgaccctgcaccacagagcaggggcgggggtgggggtggggggcagggggatgtaaaagctgaggaccatcccagcacacttgctttggagttacgtagagctccgttaggattcagtcagttttggaatggttaggggtgatttcattttttcactacatgtgggatctggcagaatcttcagtctcctggtcttgtttctggaatggagtgggtcacgaacacctcacaggagtctctcccaggagacttgagaggatcactttagaatttaagaaaacaaaaataacatgtctcactttgttcacaaagaagccctttgacaaagtggccaaaaacgggcagagcctaaggaacagactcttagggaggggtccttggaacagtatcctgcagagcgttcctctttcaaattctttagaattttacgctgttttgtggggccaccctgtcccactccctttcctaacaacacagtgcctttcttgctttgaagttgtaaactgtttttttaattgtttgcacaataaaataaagttcctcccaataagggacagactcttagggaggggtccttgggacagtatcctgcagagcgttcctctttcaaattctttagaactttacgctgttttgtggggccaccctctcccactgcccttcctaacaacacagtgcctttcttgctttgaagttgtaaactgtttttttaattgtttgcacaataaaataaagttcctcccaataagggacagactcttagggaggggtccttggaacagtgtcctgcagagtgttcctctttcaaattctttagaactttatgctgttttgtggggccaccctctcccactgcccttcctaacaacacagtgcctttcatgttttgaagttgtagactgtttgcataataaaataaagttcctcccaaatcaataatctgtaatcttaggttcttctacctgtattatccttcagcccacatttttccatttgaccatcagaaagtttacatatgtatgtacatatgtatgtacgtatgtatgtatgtacacattccctttattcaatacctatttcctgagcactctccttccctgaggacgggtctggcaccaggtagagaccagggaggtatgaagatgagtgaagcattcagtccagtgggggagacagatgtggaaacagaatcacaacacgatgtggcaaattctctgggagcacagaacaccatcactgactcttcctgatacagtcaggaaggggtgacactggaactgggtcttacaggcaagtgtcttctcctggtggaggatgagaagggcaccctggcggagggaatagcatgagctcaggtacaggggcacggaggagctgtgcatggcaggtaggacgagttgatgggggctgcagggttgcagggttcagaaaggcactaagcttaagggctgaccctggtgcttgacacatcctgcctcatttgttcctgcagcaagtctgcaagatagtagggagcaacctcaggagtcccatgtttctcctaacacagttgacatttctggatatcttaagcgccttcccaactttctccttctcccacccttattttcatttgtctattgaagatgagtccctccttccttcgccttttcactcctgcttcttaaacatatgtagagatgaaggagggttgataccagcaagctgtgaaacacaggagacttcagctgaaaaaggaggtggagaagagcagagcagaggagaccagcttgtgggaatggctgcataagttctgcagaccagcatggtgaggagtgggctccgggcatcaccaaccatgactgcctccaactatattcagcaaccattgtaaggtcatgaggatgcctttcccaaagggggaacctgctgtagatccaacccaggtttgtgaatgtaacagactggaatttttaaaagcacacagagccctttgctgcactaacagcaatggacatgtctaccaggtccaaaaaaaaaaatggtcctacacaatctgcggtggtgtattttcacacttttaataatacacacctatgcattccgtcttggggtgtgtgtgtctgtgtgtgtgtgtgtatgtgactaacttaacacactgaacgcattacaatgcattaaaaagttcttcagcagacttctctgctttagcctctgctatggtctaaatgtttatgtccccgccaaattcacttgttgaaatcctaacccccagggtgatgatggtgttagaaggtggggacatagggaggcgatcgggtcatgagggcagagccttcatgaatgggattattatccttataaaggaggagagacttccttgccccttccactgtgtaagggcacagtgaggaagcaccacctatggaccggaaagaaggccttcgctactggtgtcttgatcttggacttcccagcctccagaactgtaagcaataagtttctgttgttgttgttgtttttctgagactgagtctcgctctattgcccaggctggagtgcagtggtgtgatctcggctcactacaacctctgactcctgggttcaagtgattctcatgcctcagtttcccaattagctgggattacaggtgcgcgccgccacgccaggctaatttttgtatttttagtagagagggggtttcaccatgttggccaggctggtcttgaactcctgacctcaagtagtccacccgtctcggcctcccaaagtgctggaattacaggcatgagccagtgcgtccggccagtttctgttgtttataagccacccagttcatggtattttcttatagcagctcaaatgggctacaacagcatctctgattatcctataacaatatatccgtttctctatgggaatccatttctgtgcgagaatatgtaagaacctcagatgtttcatgccattgctgaacagaggagtctcctgggttctggacacttgccttgggaagcacatcacttggttcctctcaaacgcatttgaccactgaatagccagccactcactatggaaacacagcccgaagcactctcaatcttttggacctcagcccttttctggatctatgaaaatgtccatactggatggtgctggcaggttgcgaagtgcccaaacctcccacgcttcacaggaggaaccctctgcaaatcttacatggcaggtgggtgaccagaggagccattccaataccagtcattgctggatgtttctggggtgctgtcccaggtgctgtggagcatatctactctgtctagtctgtctttccattccagtccagtggatattgtgtcttcttcttcaggtccttgttcactgtagctgctcaggaaatagttgtgaaatggattgttgttgtggtacattcttaaagtcccttgaaagtaagaaaataaacgaatgactcttctgtgggtaaaactttggtcatcttttcacttgtttagatgagaaagaatacagaacaatgagattatagtctcctaaaacttttggaaaaattcttcatgtcctctatagtgttataattttgctattgtcccccaaagggactcaggtaacatgctttggatggaatggtgggattaaaggcacctttgattctgcacatttggtggtgttgtgaatacaaactaaacactagctgtgcagtatgcaatatttaaaggaagtagaaatcatccaaatcagcatttccagagtttttcagaacaagcaagagaatacaagagatcagcagttttctccctcaagttgttctataggaaaaaaaaaatttatccaggctcaaagaaattctataggaaggaaatctgcatacttttgctaaccccatcatttcccagactctttgaattcagtaggaagaccgcttgctccacacagttattcaaggtccccaagattcttctatctgggttttccatcatgccctagaacaatgtctttatctgcatggtcaaaactgacttacagtcgtatctgtggtcacagataggggaaggggactgaggattacatgtgcaatattttaagactcaacctgcaagtggcacacatcactttctctcacattccattagtgaaaacttaatcatgtggtcactcctcccttcaaggaggggtaggaattgtggtccctaatggggaggccatttctacattatttctgacagctacaagtctgttaatgtgtatcatagttgtcccaccagcctgcaacagagcaggggctagaagccaggtcttccaactcctagtccagagttcccattaccacactagtatttctgtacagtaagtcctcacttaatgttatcagcaggttcttggaaactgcaattttaagtgaaatcatgtataatgaaatcaattttgaaatttctcatcaatgttgtaactgagtaacgttatttgaggacctgcttacataattttgcttgaagtccaagtttccaagaacctattgatgacattaagtgaggactgactgactgtgttcagccaacatatatggagtcctcctgtgtgccaggctctatgtcaatatgagagtggaaagtcctacaaaacagaatcccctaattatatatttactctagtatcaagctttattaatggataaacgttcagatagctttgtaaagctatcccaaattatctgatagctttgcatgatgttcctttcatttgccaagaacatacatattcttggtgagggattaacacatataatgaacagagtagagaatcctgaaagagatcccaatgcatttgaaaaatgggtatatgaaaaagtcatttaaaaaatagaaccaaaaagataaatatacattaaaaaggagggtgttgagtccactaactaagaatatatatatatatatatatatatatatatatatatatatatatatagctagatttccctttaattccctcttgaagaaggaattgcttatgcaagactccaaagctgagaaccagattgtctttcagttgtcatgcagcctggaaagcccccaccttatgtaccaatagattgtgaagcaatcaaagactactacatacactttaaagccaaccagaaagactccttcccttccaagtatcttttacccgattggaactagaatatgtcaccatgaaagaacactcaccaaagattggcttaagtcttcctccttttagtctgtaaaacttactcttctaggctggcgcagtggctcacgcctataatcccagcactttgggaagctaaggcgggtggatcacctgaggtcaggagttcgagaccagcctggcaaacatggtaaaaccccgtctttactaaaaatacaaaaattagctgggagtagtggcaggtgcctgtagtcccagctgctcaggaggctgaggcaggagaatcgcttcaacctgggaagcggagcttgcagtgagctgagatcgtgctactgcactccagcctgggcaagagtgagactccatctcaaaacaaacaaacaaacaataagacaaaaaaacttactctcctttcaaaccccttccaacctctactataatgaaaatgatggcagctgactcccttataacagtaagttttgaataaacagcctttgattaattctattttgtttgccaaagaaaataattaaagtttggtaaaataaaataattaaaagaaaaagaaatcatggacactctagaagaaaacatttatttgagctttactgctgaaaaagaaaacatattttaaaaagtaaccttgactgcacagcaaaagaaactatcaacaaagtaagcagacaacctacagaatggaagaaaatatgcacaaactatgtatctgacaaaggtctaatattcagaatctataaggaacttaaatcaacaagcaaaaaacaaataaccccatttatggcctttgcttactttttgggcagtcctcaaaagaagacatccaagtggcaaacaatcatatgaaaaaatgcttatcatcactaatcatcagagaaaagcaagtcaaaaccacagtgaagtatcatcacacaccagtcaaaatggctattattaaaaataacagatgttggcagtgctgcagagaaaagggattgcttatgcactgttggtgagaatgtaaattcatttagctactgtggaaagcaggagatttctcaaagaactaaaaatatatatatcattcaaaccaacaattccattactagatatatatccaaaggaaaataaattgttctatcaaaaagacacatgcacctgtatgttcatcgcaatattattcgcaatagcaaatacatggaatcaaccaaggtgttaatcaacagaggagtggataaagaaaatgtggtacatatacaccatggaatactatacagtcagaagaaagaatgaaatcatggcctttgcagcaacatggatgcagctagggaccattatcctaagtaaactaatgcagaaacagaaaagcaaatgccacatgttctcatttataagtgggagccaagtgagtacacatggacatgaaatggaaacaatagacactggagactacaagagaagggagggggacagggattgaaaaactacctattgggtactatgctcagtacttgagtgatgggttcaatcgtatgcaaatttcagcatcatgcaatatacccttgtaacaaacctgcacatgtaccacccctgaatctaaaataaaagttgaaaaaaacaaaaacaaaaaaaccccttggaatgggaggagtcttttcagaccaaacatgagacccagaagacaaaaagtaaactgttaataagttaggctgcatagcaagccaaaatttttatatgaaacattttttaaagtgaaaagatagataaattttcaaaacatataacaaagaattaatttctttggtatacaatgagcttttaaaaattaataagaataattcaatgagaggttgaaatgggtacaaatatagagttaggtagaagcataagacctaagtgtttgatagatcagtagggtgactatagttcaagataatctattgtacctgtcaaaatagctagaagagaacaattcaaatgttcctagcattaaaaaagataaatattgccggtgatgaataccctaattactttttttttttccagacagagtctcattctgtcacccaggctgaagtgcaatggcgcaatcttggctcactgcaacctccgcctcctgggttccagtgattctcctgtctcagcctcccaagtagctgggattacaggggcccacagccatgcctggccaatttttgtatttttagcagagatggggtttcaccatgttggtcagactggtcttgaactcctgatctcaggtgatacacccacctcggcctcccaaagtgctgggatgacaagcgttagtcactgtacccgacctccttagatctttacacattatatgaatgtgtcaaattattacatgtatcccaaaaatatgtacattatgtgttaataaaaagattttcaaaattaataagaataattcaataaggaaaatggacaagggaagaaacacacaattccaggaaaaaatacaaataatcaataaatatatgatacatttaatcacatataatagaagaaatacaaataaaaataaggcacttttaacacaatctattagaataccagatatttaaatttgacagaattgagtattgttgaggatatagagaaatgggcactctcatcctacattgggagaaatataaatcaatactggattcttgagattcagtttacaataagtacaatttaagatgtgcatatctctttaccctgcaattccacttctagaaatttatcctaaatatttacttacaaatgagcaaagacatatatgtacagggatgttcagatttttatgtcgggtttttgtacggacatagttttttttttttttttttttttttttgagatggagtttcactctgtcgcccaggctggagtgcagtggcgtgatctcggctcactgcaacctccgcctcccaggttcaagcaattctcctgcctcagcctcccaagtagctgggattacaggcatgcaccaccacacctgggtagtttttctatttttagtagagacagagtttcaccatgttggccaggctggtctcgaactcccggcctcaggtgatttgcccgccatggcctcccaaagtgatgggattacaggcatgtgccaccgagcctggctatatggacatagtttttagttctttgggataaatgcccagaagtgttactgctatttaatctttaaagaaactgccaaactacttttcagagtggctgcattattttccattcccaccagcaaagtatgagtaatctatttttacgcattctttcccaaatttagtattttcacttttttttttttttttttttttttttttttttttttttttttgaggcagggtcttgctcagttgcctaggctggagtgtagtggcactatctcggctcactgcaacccctgcctcccaggcttaagcgatcctcacatctcagcaacctgagtagctggggctacaggtgagtgctaccactcatgggtaattttttttgtattcttagtagaggtggagttccgccatgttgcccaggctggtctcaaactcctgacctcaagcgatccacctgcctctgcctcccaaagtgctggggttacaggcgtgcaccactgtgcctggccattctcactatttttaattcaaaatagtttgtgttctctattctccacatctttttgttctttttaatcatctaaagtccatagtttgtattaggattaacttactgttgtacattctctctctctctctcttttttttttaatgagatggagtttctctcttttttgcccaggccaagcgcaa

SEQ ID NO: 12 - DNA, 21 bp, Artificial
  primer
tttgacttttgccccatcttc

SEQ ID NO: 13 - DNA, 20 bp, Artificial
  primer
tgcatatctgcctccaccag

SEQ ID NO: 14 - DNA, 21 bp, Artificial
  primer
gggcagaaagagatcaaggtg

SEQ ID NO: 15 - DNA, 20 bp, Artificial
  primer
tggaagaccaaccccaaatc

SEQ ID NO: 16 - DNA, 26 bp, Artificial
  primer
ctataagtcaagaaggttgtgagtgg

SEQ ID NO: 17 - DNA, 23 bp, Artificial
  primer
atctggtcatgtttgtgaagctc

(19) United States
(12) Patent Application Publication
(10) Pub. No.: US 2011/0059855 A1
(43) Pub. Date: Mar. 10, 2011

(54) Use of adiponectin for the diagnosis and/or treatment of presbycusis

(75) Inventors: Christine Petit, Le Plessis Robinson (FR); Mark Lathrop, Paris (FR); Anne Aubois, Garches (FR); Christian Kubisch, Bonn (DE); Dominique Weil, Paris (FR); Valerie Franco-Vidal, Merignac (FR); Arnaud Coez, Maisons Laffitte (FR); Jose-Alain Sahel, Paris (FR)

(73) Assignees: INSTITUT PASTEUR, Paris (FR); Institut National De La Sante Et De La Arecherche Medicale

(21) Appl. No.: 12/866,671

(22) PCT Filed: Feb. 9, 2009
(86) PCT No.: PCT/EP09/51458

§ 371 Date: Nov. 17, 2010

Related U.S. Application Data
(60) Provisional application No. 61/027,206, filed on Feb. 8, 2008.

(30) Foreign Application Priority Data
Feb. 8, 2008 (EP) 08300078.6

(51) Int. Cl. C40B 30/04; C07H 21/04; C07K 14/575

(52) U.S. Cl. 506/9; 536/23.51; 530/399; 536/24.31; 536/24.33

(57) ABSTRACT

The present invention relates to mutations located within the gene coding for adiponectin, said mutations being associated with presbycusis. The present invention further relates to adiponectin polynucleotides comprising such mutations, to adiponectin polypeptides encoded by such polynucleotides, and to methods of diagnosing and/or treating presbycusis using adiponectin polynucleotides, adiponectin polypeptides and/or ADIPOR2 polypeptides.

### FIELD OF THE INVENTION

The present invention is in the field of hearing loss, in particular presbycusis. More specifically, the present invention relates to mutations located within the gene coding for adiponectin, said mutations being associated with presbycusis. The present invention further relates to adiponectin polynucleotides comprising such mutations, to adiponectin polypeptides encoded by such polynucleotides, and to methods of diagnosing and/or treating presbycusis using adiponectin polynucleotides, adiponectin polypeptides and/or receptors binding thereto.

### BACKGROUND OF THE INVENTION

### 1. Adiponectin

Adiponectin (Swiss-Prot entry Q15848), also referred to as ACRP30, apM-1 or ADIPOQ, is an important secreted protein involved in the control of fat metabolism and insulin sensitivity, with anti-diabetic, anti-atherogenic and anti-inflammatory activities. Adiponectin stimulates AMPK phosphorylation and activation in the liver and the skeletal muscle, enhancing glucose utilization and fatty-acid combustion. Adiponectin also antagonizes TNF-alpha by negatively regulating its expression in various tissues such as liver and macrophages, and also by counteracting its effects. Adiponectin further inhibits endothelial NF-kappa-B signaling through a cAMP-dependent pathway. Adiponectin may play a role in cell growth, angiogenesis and tissue remodeling by binding and sequestering various growth factors with distinct binding affinities, depending on the type of complex.

Adiponectin is a homomultimer forming trimers, hexamers and 12- to 18-mers. The trimers (low molecular weight complexes/LMW) are assembled via non-covalent interactions of the collagen-like domains in a triple helix and hydrophobic interactions within the globular C1q domain. Several trimers can associate to form disulfide-linked hexamers (middle molecular weight complexes/MMW) and larger complexes (higher molecular weight/HMW).

Adiponectin has been shown to bind Adiponectin receptor protein 2, Adiponectin receptor protein 1 and Cadherin-13. Predicted functional partners further include the Solute carrier family 2, the facilitated glucose transporter member 4, the Leptin precursor, the CNR1 Cannabinoid receptor 1, the TNF Tumor necrosis factor precursor, the APPL DCC-interacting protein 13 alpha, Interleukin-10 and angiotensinogen.

Defects in ADIPOQ are the cause of adiponectin deficiency. The result is a very low concentration of plasma adiponectin. Decreased adiponectin plasma levels are associated with obesity insulin resistance, and diabetes type 2.

Adiponectin has been shown to potentially play a role in diseases such as diabetes, obesity, cancer, cardiovascular diseases and neurodegenerative disorders.

### 2. Presbycusis

Presbycusis, a sensorineural age-related hearing loss, affects one-third of the population after 65 years of age and 60% of the population between 70 and 80 years. Epidemiological data indicated environmental risk factors such as smoking habits, exposure to noise, to solvents and ototoxic medications. The impact of genetic factors has also been reported (Gates et al. 1999 Arch Otolaryngol Head Neck Surg. 125(6):654-9), the latter seeming to be more pronounced for individuals below 64 years.

Although there appears to be a genetic basis for the disease, very few genetic studies on presbycusis have been carried out. Genome-wide linkage studies resulted in a total of seven different candidate genomic regions for susceptibility to presbycusis, all with lod score inferior to 3, and no identified gene (DeStefano et al. Arch Otolaryngol Head Neck Surg. 2003 March; 129(3):285-9; Garringer et al. Arch Otolaryngol Head Neck Surg. 2006 May; 132(5):506-10).

Association studies have also been carried out (Unal et al. Laryngoscope. 2005 December; 115(12):2238-41; Van Eyken et al. Audiol Neurootol. 2007; 12(6):345-58; Van Eyken et al. Hum Mutat. 2006 October; 27(10):1007-16; O'Grady et al. Int J. Audiol. 2007 April; 46(4):183-6; Van Laer et al. Hum Mol Genet. 2008 Jan. 15;17(2):159-69). NAT2, an enzyme thought to be involved in the detoxification of exogenic substrates by N— or O— acetylation, is the only gene which has been independently replicated so far.

There is thus a need to identify genes and mutations therein leading to a susceptibility to presbycusis, both in order to diagnose the disease and in order to develop powerful treatments.

### SUMMARY OF THE INVENTION

It has surprisingly been found that the chromosomal region located between 187910000 and 189120000 of human chromosome 3 and comprising mutations compared to the sequence found in normal subjects is associated with presbycusis with a highly significant lod score of 3.4. Three mutations have been found within this region. These mutations are located within the gene coding for adiponectin, at positions 4988, 15361 and 16514 of SEQ ID NO: 1 respectively. An additional genetic analysis carried out on 113 individuals suffering from presbycusis allowed the identification of three additional mutations. These mutations are shown in the table below:

| | | Mutation found in |
|---|---|---|
| Position on SEQ ID | Nucleotide found | individuals suffering |
| NO: 1 | in normal subjects | from presbycusis |
| 4988 | T | C |
| 15361 | T | A |
| 16514 | G | A |
| 15380 | T | G |
| 16577 | T | C |
| 16746 | A | G |

The alleles comprising these mutations code for novel, mutated adiponectin polypeptides. These mutations are predicted to lead to impaired multimerization and/or impaired secretion of adiponectin. Reduced levels of adiponectin are therefore believed to cause presbycusis.

The invention is directed to an isolated, purified or recombinant adiponectin polynucleotide comprising a sequence at least 80% identical to SEQ ID NO: 1, or to a fragment thereof, characterized in that said polynucleotide comprises a cytosine at position 4988 of SEQ ID NO: 1, an adenosine at position 15361 of SEQ ID NO: 1, a guanosine at position 15380 of SEQ ID NO: 1 and/or a guanosine at position 16746 of SEQ ID NO: 1.

The invention is further directed to an isolated, purified or recombinant adiponectin polypeptide comprising a sequence at least 80% identical to SEQ ID NO: 2, or to a fragment thereof, characterized in that said polypeptide comprises a glutamine at amino acid position 9 of SEQ ID NO: 2 and/or a cysteine at position 167 of SEQ ID NO: 2.

Another aspect of the invention is directed to the use of at least one adiponectin-related allelic marker for determining whether there is a genetic association between said allelic marker and presbycusis.

Still another aspect of the invention is directed to the use of at least one adiponectin-related allelic marker for diagnosing whether an individual suffers from or is at risk of suffering from presbycusis.

The invention is also directed to a method of genotyping comprising the steps of:
a) obtaining an isolated nucleic acid from a biological sample derived from an individual; and
b) detecting the nucleotide present at one or more of the adiponectin-related allelic markers of the invention shown in the table below:

| Allelic marker | Position on SEQ ID | |
|---|---|---|
| No. | NO: 1 | Alternative alleles |
| 1 | 4988 | T/C |
| 2 | 15361 | T/A |
| 3 | 16514 | G/A |
| 4 | 15380 | T/G |
| 5 | 16577 | T/C |
| 6 | 16746 | A/G |

The invention further pertains to probes comprising a fragment of the adiponectin gene, wherein said probe comprises allelic marker NO: 1, 2, 4 or 6 of the invention, and to primers comprising a fragment of the adiponectin gene, wherein the 3′ end of said primer is located in the vicinity of, preferably within 500 nucleotides, of allelic marker NO: 1, 2, 4 or 6 of the table hereabove.

The invention also encompasses a pair of primers comprising a first and a second primer each comprising a fragment of an adiponectin gene, wherein:
said first primer hybridizes to a first DNA strand of said adiponectin gene;
said second primer hybridizes to the strand complementary to said first DNA strand; and
the 3′ ends of said first and second primers are located in the vicinity of allelic marker NO: 1, 2, 4 or 6 of the table hereabove.

The invention is further drawn to a method of identifying an ADIPOR2-related allelic marker likely to be involved in presbycusis comprising the steps of:
determining the sequence of the ADIPOR2 gene present in biological samples from at least one individual suffering from presbycusis and from at least one unaffected individual; and
comparing said sequences;
   wherein a difference in said sequences is identified as an ADIPOR2-related allelic marker.

Another aspect of the invention is directed to an adiponectin polynucleotide of the invention, an adiponectin polypeptide of the invention or a fragment or an agonist thereof for use in the treatment of presbycusis.

The invention also relates to a method of determining whether an individual suffers from or is at risk of suffering from presbycusis comprising the steps of:
a) providing a biological sample from said individual; and
b) comparing:
   i. the amount of adiponectin polypeptide, or of an RNA species encoding said polypeptide, within said biological sample with an amount detected in or expected from a control sample; or
   ii. the activity of adiponectin polypeptide within said biological sample with an activity detected in or expected from a control sample;
      wherein a reduced amount or activity of said adiponectin polypeptide or said RNA species within said biological sample compared to said level detected in, or expected from, said control sample indicates that said individual suffers from or is at risk of suffering from presbycusis.

### BRIEF DESCRIPTION OF THE FIGURE

FIG. 1 shows the results of the immunochemistry experiments described in Example 5. A. p7 mouse auditory sensory cells stained with an anti-actin antibody (1^{st} panel) or with a monoclonal anti-adiponectin antibody (2^{nd} panel). The three first panels show views taken from above, and the last panel shows a profile view. B. p7 mouse auditory sensory cells stained with an anti-actin antibody (1^{st} panel) or with a polyclonal anti-adiponectin antibody (2^{nd} panel). The three first panels show views taken from above, and the last panel shows a profile view. C. p7 mouse auditory sensory cells stained with an anti-actin antibody (1^{st} panel) or with a polyclonal anti-ADIPOR1 antibody (2^{nd} panel).

### BRIEF DESCRIPTION OF THE SEQUENCES

SEQ ID NO: 1—Nucleotide sequence of part of the gene coding for adiponectin. SEQ ID NO: 1 comprises the promoter, the introns and the exons of the adiponectin gene.

SEQ ID NO: 2—Amino acid sequence of an adiponectin polypeptide.

SEQ ID NO: 3—Amino acid sequence of the ADIPOR2 polypeptide.

SEQ ID NOs: 4-9—Nucleotide sequences of primers used for amplifying fragments of the adiponectin gene.

SEQ ID NO: 10—Nucleotide sequence of the gene coding for ADIPOR2.

SEQ ID NO: 11—Nucleotide sequence of the regulatory region of the adiponectin gene (upstream of the ATG codon).

SEQ ID NOs: 12-17—Nucleotide sequences of primers used for amplifying fragments of the adiponectin gene.

### DETAILED DESCRIPTION OF THE INVENTION

It has surprisingly been found that the chromosomal region located between 187910000 and 189120000 of chromosome 3 and comprising mutations compared to the sequence found in normal subjects is associated with presbycusis with a highly significant lod score of 3.4. The lod score is a statistical test used for linkage analysis. A lod score greater than 3.0 means that the likelihood of linkage between two allelic markers or a trait and a marker is 1000 times greater than no linkage.

Three mutations have been found. These mutations are located within the gene coding for adiponectin, at positions 4988, 15361 and 16514 of SEQ ID NO: 1 respectively.

An additional genetic analysis of 113 patients suffering from presbycusis allowed the identification of three further mutations within the adiponectin gene, found in patients suffering from presbycusis. More specifically, these mutations are as follows:

| | | Mutation found in |
|---|---|---|
| Position on | Nucleotide found in | individuals suffering |
| SEQ ID NO: 1 | normal subjects | from presbycusis |
| 4988 | T | C |
| 15361 | T | A |
| 16514 | G | A |
| 15380 | T | G |
| 16577 | T | C |
| 16746 | A | G |

The alleles comprising these mutations code for novel, mutated adiponectin polypeptides. These mutations are predicted to lead to impaired multimerization and/or impaired secretion of adiponectin. Reduced levels of adiponectin are therefore believed to cause presbycusis.

Therefore, a first aspect of the invention is directed to an isolated, purified or recombinant adiponectin polynucleotide comprising or consisting of a sequence at least 80%, 85%, 90%, 95%, 96%, 97%, 98% or 99% identical to SEQ ID NO: 1, or to a fragment of such a sequence, characterized in that said polynucleotide comprises a cytosine at position 4988 of SEQ ID NO: 1, an adenosine at position 15361 of SEQ ID NO: 1, a guanosine at position 15380 of SEQ ID NO: 1 and/or a guanosine at position 16746 of SEQ ID NO: 1. The adiponectin polynucleotide may further comprise an adenosine at position 16514 of SEQ ID NO: 1 and/or a cytosine at position 16577 of SEQ ID NO: 1.

As used herein, the term “adiponectin polynucleotide” refers to the adiponectin gene (including the 5′ regulatory region, the promoter, the introns, the exons and the 3′ regulatory region) and to fragments thereof. The adiponectin gene is located on chromosome 3 at 3q27, and is shown in Genbank accession number NC_{—}000003.10 (188043157.188058946). SEQ ID NOs: 1 and 11 correspond to adiponectin polynucleotides. SEQ ID NO: 1 comprises the promoter, the introns and the exons of the adiponectin gene. SEQ ID NO: 11 corresponds to the regulatory region of the adiponectin gene (upstream of the ATG codon). The fragments may be of any length, e.g. at least 10, 25, 50, 100, 500 or 1000 nucleotides long. In one embodiment, the adiponectin polynucleotide encodes an adiponectin polypeptide. Adiponectin polynucleotides in accordance with the invention comprise a cytosine at position 4988 of SEQ ID NO: 1, an adenosine at position 15361 of SEQ ID NO: 1, a guanosine at position 15380 of SEQ ID NO: 1 and/or a guanosine at position 16746 of SEQ ID NO: 1.

In a preferred embodiment, the adiponectin polynucleotide comprises or consists of SEQ ID NO: 1, wherein said polynucleotide comprises a cytosine at position 4988 of SEQ ID NO: 1, an adenosine at position 15361 of SEQ ID NO: 1, a guanosine at position 15380 of SEQ ID NO: 1 and/or a guanosine at position 16746 of SEQ ID NO: 1.

In another preferred embodiment, the adiponectin polynucleotide further comprises SEQ ID NO: 11 or a fragment thereof, wherein said polynucleotide comprises a cytosine at position 4988 of SEQ ID NO: 1, an adenosine at position 15361 of SEQ ID NO: 1, a guanosine at position 15380 of SEQ ID NO: 1 and/or a guanosine at position 16746 of SEQ ID NO: 1.

A second aspect of the invention is directed to an isolated, purified or recombinant adiponectin polypeptide comprising or consisting of a sequence at least 80%, 85%, 90%, 95%, 96%, 97%, 98% or 99% identical to SEQ ID NO: 2, or to a fragment of such a sequence, characterized in that said polypeptide comprises a glutamine at amino acid position 9 of SEQ ID NO: 2 and/or a cysteine at position 167 of SEQ ID NO: 2. This polypeptide may further comprise a serine at amino acid position 90 of SEQ ID NO: 2 and/or an histidine at position 111 of SEQ ID NO: 2.

The term “adiponectin polypeptide” refers to a polypeptide of SEQ ID NO: 2 and to splice variants, allelic variants, fragments, muteins and functional derivatives thereof having adiponectin biological activity. Specifically, the term “adiponectin polypeptide” includes polypeptides in which one or more of the amino acid residues of an adiponectin polypeptide of SEQ ID NO: 2 are replaced by different amino acid residues, or are deleted, or one or more amino acid residues are added to the wild-type sequence.

As used herein, the term “wild type adiponectin polypeptide” refers to a polypeptide of SEQ ID NO: 2 wherein said polynucleotide comprises a leucine at position 9 of SEQ ID NO: 2, a glycine at position 90 of SEQ ID NO: 2, a tyrosine at position 111 of SEQ ID NO: 2, and a tyrosine at position 167 of SEQ ID NO: 2. Adiponectin polypeptides in accordance with the invention comprise a glutamine at amino acid position 9 of SEQ ID NO: 2 and/or a cysteine at position 167 of SEQ ID NO: 2. Fragments of adiponectin polypeptides may have any lengths, e.g. be at least 10, 20, 30, 50, 75, 100, 150 or 200 amino acids.

As used throughout this specification, the term “biological activity” of an adiponectin polypeptide refers to any of the known biological activities of adiponectin, such as e.g. an anti-diabetic, anti-obesity, anti-thrombotic, anti-coagulant and/or anti-aggregant activity. An adiponectin polypeptide has biological activity as soon as it exhibits some biological activity as described hereabove, although the level may be reduced or impaired as compared to a wild-type adiponectin polypeptide. Methods of testing the biological activity of an adiponectin polypeptide are well-known in the art and include e.g. the methods disclosed in WO 01/51645 and WO 2007/147563.

A third aspect of the invention is directed to an expression vector comprising an adiponectin polynucleotide of the invention. Preferably, such an expression vector encodes an adiponectin polypeptide of the invention. Such expression vectors of the invention may be used e.g. to produce adiponectin polypeptides, or may correspond to vectors suitable for carrying out a gene therapy.

A fourth aspect of the invention is directed to a host cell comprising an expression vector of the invention. The host cell may correspond to any cell suitable for producing proteins or for maintaining viral vectors. Such cells may be e.g. a bacterial (e.g. E. coli, S. lividans), a yeast (e.g. S. cerevisiae), a fungal (e.g. A. niger), an insect, a mouse, a CHO, a monkey or a human cell.

A fifth aspect of the invention is directed to a method of making a polypeptide, said method comprising the steps of culturing a host cell of the invention under conditions suitable for the production of an adiponectin polypeptide of the invention within said host cell. Such conditions are well-known in the art. Preferably, the produced polypeptide is further purified. Most preferably, the polypeptide is further formulated into a pharmaceutical composition.

Since the chromosomal region located between 187910000 and 189120000 of chromosome 3 and comprising mutations compared to the wild-type sequence is associated with presbycusis with a highly significant lod score of 3.4, a sixth aspect of the invention is directed to the use of at least one adiponectin-related allelic marker for determining whether there is a genetic association between said allelic marker and presbycusis.

As used herein, the term “adiponectin-related allelic marker” refers to an allelic marker located within the adiponectin gene. Such an allelic marker may be located within the 5′ or 3′ regulatory gene regions, the promoter, the exons (including the 5′ and the 3′ UTRs) or the introns of the adiponectin gene. Preferably, such an allelic marker is located within SEQ ID NO: 1 or SEQ ID NO: 11. Most preferably, the adiponectin-related allelic marker is selected from the allelic markers shown in Table 1 herebelow:

TABLE 1

| Allelic marker | Position on SEQ ID | |
|---|---|---|
| No. | NO: 1 | Alternative alleles |
| 1 | 4988 | T/C |
| 2 | 15361 | T/A |
| 3 | 16514 | G/A |
| 4 | 15380 | T/G |
| 5 | 16577 | T/C |
| 6 | 16746 | A/G |

As used herein, the term “allelic marker” refers to a readily identifiable genetic element that exists either in a wild-type form or in a mutated form and is associated with a genetic trait of interest. In the frame of this invention, the genetic element corresponds to the existence of a mutation and encompasses e.g. substitutions, deletions and insertions of a nucleotide.

As used herein, there is a “genetic association” between an allelic marker or a combination of allelic markers and a disease when a particular allele, genotype or haplotype of said allelic marker or combination of allelic markers is seen more often than expected by chance in an individual carrying the disease.

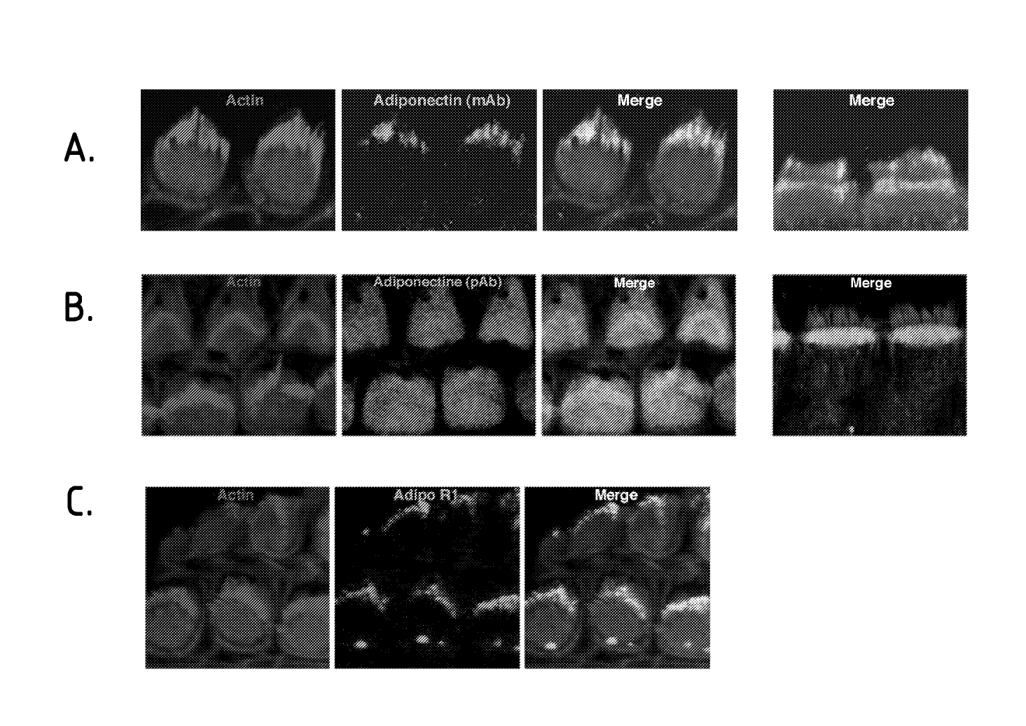

As used herein, the term “presbycusis” refers to age-related hearing loss, and encompasses all conditions that lead to hearing loss in elderly people (see e.g. Gates and Mills, Lancet. 2005 September 24-30;366(9491):1111-20). The disorder is characterized by reduced hearing sensibility and speech understanding in noisy environment, slowed central processing of acoustic information, and/or impaired localisation of acoustic sources. An individual is considered to suffer from presbycusis when its hearing threshold is increased above the threshold values shown in FIG. 1. The presbycusis may correspond to a “familial case of presbycusis”, i.e. to an inheritable form of the disease. Preferably, an individual being a familial case of presbycusis satisfies the criteria of having (i) at least one affected brother or sister; and (ii) one or none affected mother or father. Alternatively, the presbycusis may correspond to a sporadic case of presbycusis, i.e. none of the brothers and sisters of the affected individual suffers from presbycusis.

Methods for determining whether there is a genetic association between said allelic marker and presbycusis are well-known in the art.

A preferred embodiment is directed to a method of determining whether there is a genetic association between at least one adiponectin-related allelic marker and presbycusis comprising the steps of:
a) providing biological samples from individuals suffering from presbycusis and from unaffected individuals;
b) determining the identity of a nucleotide present at said allelic marker; and
c) comparing the allele frequency of said allelic marker in individuals suffering from presbycusis with the allele frequency of said allelic marker in unaffected individuals.
   wherein a difference in the allele frequency of said allelic marker in individual suffering from presbycusis and in unaffected individuals is indicative of a genetic association between said allelic marker and presbycusis.

The expression “identity of a nucleotide present at a allelic marker” refers to the nucleotide present at said allelic marker and may correspond to A (adenosine), C (cytosine, also referred to as cytidine), G (guanosine) or T (thymidine).

Preferably, biological samples are collected from at least 2, 5, 10, 25, 50, 75 or 100 individuals suffering from presbycusis. More preferably, biological samples are collected from at least 5, 10, 25, 50, 75 or 100 unaffected individuals. Most preferably, the allele frequencies are statistically compared in step (c).

As the adiponectin allele comprising mutations at positions 4988, 15361 and/or 16514 of SEQ ID NO: 1 is associated with presbycusis, the invention further relates to methods for diagnosing presbycusis.

A seventh aspect of the invention is directed to the use of at least one adiponectin-related allelic marker for diagnosing whether an individual suffers from or is at risk of suffering from presbycusis. Preferably, said adiponectin-related allelic marker is selected from the allelic markers shown in Table 1 hereabove.

In a specific embodiment, the detection of a mutated adiponectin allele comprising a C at position 4988, an A at position 15361 and/or an A at position 16514 of SEQ ID NO: 1 in a biological sample collected from an individual indicates that said individual suffers from or is at risk of suffering from presbycusis.

In a further specific embodiment, the detection of a mutated adiponectin allele comprising a G at position 15380, a C at position 16577 and/or a G at position 16746 of SEQ ID NO: 1 in a biological sample collected from an individual indicates that said individual suffers from or is at risk of suffering from presbycusis.

An eight aspect of the invention is directed to method of genotyping comprising the steps of:
a) obtaining an isolated nucleic acid from a biological sample derived from an individual; and
b) detecting the nucleotide present at one or more of the adiponectin-related allelic markers shown in table 1 hereabove.

Preferably, said biological sample is derived from a single individual. It is preferred that the nucleotide present at one or more of said adiponectin-related allelic markers is detected for both copies of said allelic markers present in said individual's genome. In a preferred embodiment, the nucleotide present at one or more of said adiponectin-related allelic markers is detected by a sequencing assay. Preferably, a portion of a sequence comprising the allelic marker is amplified prior to the determination of the identity of the nucleotide. The amplification may preferably be performed by PCR.

Any well-known method of genotyping may be used in the frame of the present invention. Such methods include methods such as e.g. conventional dot blot analyzes, single strand conformational polymorphism analysis, denaturing gradient gel electrophoresis, heteroduplex analysis and mismatch cleavage detection. Another method for determining the identity of the nucleotide present at a particular polymorphic site employs a specialized exonuclease-resistant nucleotide derivative as described in U.S. Pat. No. 4,656,127. Oligonucleotide microarrays or solid-phase capturable dideoxynucleotides and mass spectrometry may also be used. Preferred methods involve directly determining the identity of the nucleotide present at an allelic marker site by sequencing assay, enzyme-based mismatch detection assay, or hybridization assay.

The term “qenotypinq” a sample or an individual for an allelic marker involves determining the specific allele or the specific nucleotide carried by an individual at an allelic marker.

In a preferred embodiment, the method of genotyping of the invention further comprises the step of correlating the result of the genotyping steps with a risk of suffering from presbycusis and/or the step of diagnosing presbycusis.

In a more preferred embodiment, the presence of:
a) a C at position 4988 of SEQ ID NO: 1;
b) an A at position 15361 of SEQ ID NO: 1;
c) an A at position 16514 of SEQ ID NO: 1;
d) a G at position 15380 of SEQ ID NO: 1;
e) a C at position 16577 of SEQ ID NO: 1; and/or
f) a G at position 16746 of SEQ ID NO: 1;
   indicates that said individual suffers from or is at risk of suffering from presbycusis.

In a more preferred embodiment, the presence of a C at position 4988, an A at position 15361 and an A at position 16514 of SEQ ID NO: 1 indicates that said individual suffers from or is at risk of suffering from presbycusis.

The present invention is also directed to a method of identifying an adiponectin-related allelic marker likely to be involved in presbycusis comprising the steps of:
determining the sequence of an adiponectin gene or a fragment thereof present in said samples from at least one individual suffering from presbycusis and from at least one unaffected individual; and
comparing said sequences;
   wherein a difference in said sequences is identified as an adiponectin-related allelic marker.

The difference preferably corresponds to a mutation present in said at least one individual suffering from presbycusis. More preferably, the difference corresponds to a mutation that is more frequently found in individuals suffering from presbycusis than in unaffected individuals. Most preferably, said sequence is determined for the region corresponding to SEQ ID NO: 1 and/or SEQ ID NO: 11, and the identified adiponectin-related allelic marker is located within SEQ ID NO: 1 or SEQ ID NO: 11.

The invention further relates to probes and primers useful for detecting the mutated adiponectin alleles of the invention. The probes and primers according to the invention may be of any length, e.g. at least 10, 12, 15, 20, 50, 100, 200, 300, 400 or 500 nucleotides long.

A ninth aspect of the invention is therefore directed to an isolated, purified or recombinant primer or probe comprising a fragment of the adiponectin gene, wherein said primer or probe comprises allelic marker NO: 1, 2, 4 or 4 of table 1 hereabove. Said probe preferably comprises a cytosine at position 4988 of SEQ ID NO: 1, an adenosine at position 15361 of SEQ ID NO: 1, a guanosine at position 15380 of SEQ ID NO: 1 and/or a guanosine at position 16746 of SEQ ID NO: 1.

Said fragment of an adiponectin gene may comprise or consist of a contiguous span of at least 12, 15, 20, 25, 30, 35, 40, 45, 50, 60, 70, 80, 90 or 100 nucleotides of SEQ ID NO: 1 or the complementary sequence thereof. The length of said primer or probe may be within a range of 12-100, 15-90, 20-80, 25-70, 30-60, 35-50 and 15-50 nucleotides.

Such primers or probes are used e.g. in methods of determining whether an individual carries a C at position 4988, an A at position 15361, a guanosine at position 15380 of SEQ ID NO: 1 and/or a guanosine at position 16746 of SEQ ID NO: 1. For example, the probe may be used in mismatch cleavage detection or in hybridization assays for genotyping an adiponectin-related allelic marker of the invention.

In a preferred embodiment, the contiguous span of said primer or probe is 12 to 50 nucleotides in length and said allelic marker is within 4 nucleotides of the centre of said primer or probe.

In another preferred embodiment, said primer or probe consists of said contiguous span and said contiguous span is 25 nucleotides in length and said allelic marker is at the centre of said primer or probe.

In still another preferred embodiment, said allelic marker is present at the 3′ end of said primer or probe. In the context of this embodiment, the 3′ end of said contiguous span is preferably located at the 3′ end of said probe.

A tenth aspect of the invention is directed to an isolated, purified or recombinant primer comprising a fragment of the adiponectin gene. Preferably, the primer comprises or consists of a fragment of SEQ ID NO: 1 or SEQ ID NO: 11. Most preferably, the 3′ end of said primer is located in the vicinity of allelic marker NO: 1, 2, 4 or 6 of table 1 hereabove. The primers may or may not comprise one of the allelic markers 1 to 6 of table 1 hereabove.

The 3′ end of a primer is located “in the vicinity” of an allelic marker if said 3′ end is located within 4000, 3000, 2000, 1000, 500, 400, 300, 250, 200, 100, 50, 25, 10 or 1 nucleotides of said allelic marker. The 3′ end of said primer is preferably located upstream (i.e. toward the 5′ end) of said biallelic marker. Alternatively, the primer may comprise the biallelic marker.

Said fragment of an adiponectin gene may comprise or consist of a contiguous span of at least 12, 15, 20, 25, 30, 35, 40, 45, 50, 60, 70, 80, 90 or 100 nucleotides of SEQ ID NO: 1 or SEQ ID NO: 11 or the complementary sequence thereof. The length of said primer may be within a range of 12-100, 15-90, 20-80, 25-70, 30-60, 35-50 and 18-25 nucleotides.

Such primers are used e.g. in methods of determining whether an individual carries a C at position 4988, an A at position 15361, a guanosine at position 15380 of SEQ ID NO: 1 and/or a guanosine at position 16746 of SEQ ID NO: 1. For example, the primer may be used in sequencing assays for genotyping an adiponectin-related allelic marker of the invention or in PCR reactions for amplifying a fragment of an adiponectin polynucleotide comprising an allelic marker of the invention.

In a preferred embodiment, the 3′ end of said primer is located within 1 nucleotide of said allelic marker.

In another preferred embodiment, said primer is selected from the group consisting of SEQ ID NOs: 4-7 and 12-17. Such primers typically find use in PCR reactions for amplifying a fragment of an adiponectin polynucleotide comprising allelic marker NO:1, 2, 4 or 6 of the invention.

An eleventh aspect of the invention is directed to a pair of primers comprising a first and a second primer each comprising a fragment of an adiponectin gene, wherein:
said first primer hybridizes to a first DNA strand of said adiponectin gene;
said second primer hybridizes to the strand complementary to said first DNA strand; and
the 3′ ends of said first and second primers are located in the vicinity of allelic marker NO: 1, 2, 4 or 6 of table 1 hereabove.

In a preferred embodiment; said pair of primers comprises:
a first primer comprising a contiguous span of at least 12, 15, 20, 25, 30, 35, 40, 45, 50, 60, 70, 80, 90 or 100 nucleotides of SEQ ID NO: 1; and
a second primer comprising a contiguous span of at least 12, 15, 20, 25, 30, 35, 40, 45, 50, 60, 70, 80, 90 or 100 nucleotides of a sequence complementary SEQ ID NO: 1.

The 3′ ends of said first and second primers are preferably located within 500, 400, 300, 250, 200, 100 or 50 nucleotides of the allelic marker. Said first and second primers are preferably located upstream of the biallelic marker. The length of said primer may be within a range of 12-100, 15-90, 20-80, 25-70, 30-60, 35-50 and 18-25 nucleotides.

In a preferred embodiment, said pair of primers consists of SEQ ID NOs: 4 and 5, of SEQ ID NOs: 6 and 7, of SEQ ID NOs: 12 and 13, of SEQ ID NOs: 14 and 15, or of SEQ ID NOs: 16 and 17.

Such pairs of primers are used e.g. in methods of determining whether an individual carries a C at position 4988, an A at position 15361, a G at position 15380 and/or a G at position 16746 of SEQ ID NO: 1. Typically, such pairs of primers are used in PCR reactions for amplifying a fragment of an adiponectin polynucleotide comprising allelic marker NO: 1, 2, 4 or 6 of the invention.

The primers of the present invention may optionally comprise sequences in addition to the contiguous span of at least 12 nucleotides of SEQ ID NO: 1 or the sequence complementary thereto such as e.g. a restriction site. In this case, the contiguous span homologous to SEQ ID NO: 1 is located at the 3′ end of said primer.

The primers are optionally labelled. They may be labelled either with a radioactive or with a non-radioactive compound such as e.g. biotin.

The invention also pertains to the use of a primer or probe in accordance with the invention for determining the identity of the nucleotide present at least one adiponectin-related allelic marker in accordance with the invention, and to a method for determining the identity of the nucleotide present at least one adiponectin-related allelic marker according to the invention comprising the steps of:
i. providing a biological sample from a patient suffering from, or susceptible of suffering from, presbycusis;
ii. determining the identity of a nucleotide present at said at least one adiponectin-related allelic marker according to the invention.

Preferably, said at least one adiponectin-related allelic marker according to the invention corresponds to the adiponectin-related allelic marker No. 1, 2, 4 and/or 6.

A twelfth aspect of the invention is directed to a microarray comprising at least one probe according to the invention or at least a primer according to the invention.

A thirteenth aspect of the invention is directed to a diagnostic kit comprising at least one probe according to the invention or at least a primer according to the invention.

ADIPOR2 and ADIPOR1 being receptors for adiponectin, a fourteenth aspect of the invention is directed to a method of identifying an ADIPOR1 or ADIPOR2-related allelic marker likely to be involved in presbycusis comprising the steps of:
providing biological samples from at least one individual suffering from presbycusis and from at least one unaffected individual;
determining the sequence of an ADIPOR1 or ADIPOR2 gene or a fragment thereof present in said samples; and
comparing said sequences;
   wherein a difference in said sequences is identified as an ADIPOR1 or ADIPOR2-related allelic marker. The difference preferably corresponds to a mutation present in said at least one individual suffering from presbycusis. More preferably, the difference corresponds to a mutation that is more frequently found in individuals suffering from presbycusis than in unaffected individuals.

Preferably, said biological samples are collected from at least 5, 10, 25, 50, 75 or 100 individual suffering from presbycusis and from at least 5, 10, 25, 50, 75 or 100 unaffected individual.

As used herein, the term “ADIPOR2” refers to the Progestin and adipoQ receptor family member II, the sequence of which is shown as SEQ ID NO: 3. An “ADIPOR2-related allelic marker” refers to an allelic marker that is located within the ADIPOR2 gene. The term “ADIPOR2 gene” refers to the gene encoding ADIPOR2, including the 5′ and 3′ regulatory regions, the promoter, the exons and the introns. The ADIPOR2 gene is located on chromosome 12 at 12p13.31. The sequence of the ADIPOR2 gene is shown as SEQ ID NO: 10 and in Genbank accession number NC_{—}000012.10 (1670508.1768106).

As used herein, the term “ADIPOR1” refers to the Progestin and adipoQ receptor family member I. An “ADIPOR1-related allelic marker” refers to an allelic marker that is located within the ADIPOR1 gene. The term “ADIPOR1 gene” refers to the gene encoding ADIPOR1, including the 5′ and 3′ regulatory regions, the promoter, the exons and the introns. The ADIPOR1 gene is located on chromosome 1 at 1p36.13-q41. The sequence of the ADIPOR1 gene is shown in Genbank accession number NC_{—}000001.9.

A further embodiment of the invention is directed to a method of determining whether there is a genetic association between at least one ADIPOR1 or ADIPOR2-related allelic marker and presbycusis comprising the steps of:
a) providing biological samples from individuals suffering from presbycusis and from unaffected individuals;
b) determining the identity of a nucleotide present at said allelic marker; and
c) comparing the allele frequency of said allelic marker in individual suffering from presbycusis with the allele frequency of said allelic marker in unaffected individual.
   wherein a difference in the allele frequency of said allelic marker in individual suffering from presbycusis and in unaffected individuals is indicative of a genetic association between said allelic marker and presbycusis.

Preferably, said biological samples are collected from at least 2, 5, 10, 25, 50, 75 or 100 individual suffering from presbycusis and from at least 2, 5, 10, 25, 50, 75 or 100 unaffected individual.

Since reduced adiponectin levels are believed to cause presbycusis, a fifteenth aspect of the invention is directed to an adiponectin polynucleotide of the invention for use in the treatment of presbycusis. In the context of this aspect, the adiponectin polynucleotide is typically present on a gene therapy vector, and is administrated to the individual in the frame of a gene therapy. Also in the context of this aspect, the adiponectin polynucleotide corresponds to a functional (wild-type) adiponectin polynucleotide. It preferably comprises a tyrosine at position 4988, a tyrosine at position 15361, and a guanosine at position 16514 of SEQ ID NO: 1.

A preferred embodiment is directed to a method of treating presbycusis comprising the step of administering an adiponectin polynucleotide to a patient suffering from presbycusis.

A sixteenth aspect of the invention is directed to an adiponectin polypeptide or a fragment or an agonist thereof of the invention for use in the treatment of presbycusis. In the frame of this aspect, the adiponectin polypeptide corresponds to a functional (wild-type) adiponectin polypeptide. It preferably comprises leucine at position 9 of SEQ ID NO: 2, a glycine at position 90 of SEQ ID NO: 2, a tyrosine at position 111 of SEQ ID NO: 2, and a tyrosine at position 167 of SEQ ID NO: 2.

An “agonist of adiponectin” refers to a compound that increases the activity of an adiponectin polypeptide in a cell or in an individual, a compound increasing adiponectin plasma levels in an individual, and/or a compound that increases the expression level of the adiponectin mRNA in a cell. The agonist may be e.g. a natural ligand, a small molecule, an agonistic antibody or an aptamer. Adiponectin agonists are known in the art and include e.g. thiazolidinediones (Iwaki et al. Diabetes. 2003 July; 52(7):1655-63), gliclazide

(Drzewoski and Zurawska-Klis, Curr Med Res Opin. 2006 October; 22(10):1921-6), quinapril (Hermann et al. J Clin Endocrinol Metab. 2006 March; 91(3):1001-8) and rosiglitazone (Yang et al. Diabetes Care. 2002 February; 25(2):376-80),

In the frame of this aspect of the invention, a “fragment of adiponectin” refers to a fragment of an adiponectin polypeptide having adiponectin biological activity. Such fragments are known in the art and may e.g. correspond to a globular Adiponectin, i.e. to a fragment of Adiponectin comprising the C1q domain but not the collagen domain. The globular Adiponectin may for example correspond to any of the globular Adiponectins disclosed in Fruebis et al. (Proc Natl Acad Sci USA. 2001 Feb. 13; 98(4):2005-10), WO 01/92330 or WO 01/51645. The fragment may for example have a length of at least 10, 20, 30, 50, 75, 100, 150 or 200 amino acids.

A preferred embodiment is directed to a method of treating presbycusis comprising the step of administering an adiponectin polypeptide or a fragment or an agonist thereof to a patient suffering from presbycusis.

As ADIPOR1 and ADIPOR2 are receptors for adiponectin, a seventeenth aspect of the invention is directed to an ADIPOR1 or an ADIPOR2 agonist for use in the treatment of presbycusis. The agonist may be e.g. a natural ligand, a small molecule, an agonistic antibody or an aptamer.

One example of such an ADIPOR1 or ADIPOR2 agonist is its natural ligand Adiponectin.

A preferred embodiment is directed to a method of treating presbycusis comprising the step of administering an ADIPOR1 or ADIPOR2 agonist to a patient suffering from presbycusis.

An eighteenth aspect of the invention is directed to a method of determining whether an individual suffers from or is at risk of suffering from presbycusis comprising the steps of:
a) providing a biological sample from said individual;
b) comparing:
   i. the amount of adiponectin polypeptide, or of an RNA species encoding said polypeptide, within said biological sample with an amount detected in or expected from a control sample; or
   ii. the activity of adiponectin polypeptide within said biological sample with an activity detected in or expected from a control sample;
      wherein a reduced amount or activity of said adiponectin polypeptide or said RNA species within said biological sample compared to said level detected in or expected from said control sample indicates that said individual suffers from or is at risk of suffering from presbycusis.

The above method may be useful e.g. for diagnosing presbycusis, for assessing the severity of presbycusis in an individual, for designing a treatment regimen, for monitoring the progression of presbycusis in a patient, for monitoring the response of a patient to a drug and/or for adjusting the treatment of a patient. It may be carried out at least at two different points in time for a given individual, e.g. before and after onset of a therapeutic treatment, or at regular intervals in the frame of a long-term treatment.

All references cited herein, including journal articles or abstracts, published or unpublished patent application, issued patents or any other references, are entirely incorporated by reference herein, including all data, tables, figures and text presented in the cited references.

Although having distinct meanings, the terms “comprising”, “having” and “consisting of” have been used interchangeably throughout this specification and may be replaced with one another.

The invention will be further evaluated in view of the following examples and figures.

### EXAMPLES

### Example 1

### Enrolment of Patients Suffering from Presbycusis

The inclusion criteria for patients suffering from familial cases of presbycusis were the following:

1. Clinical Criteria:
40 years old at least at the time of diagnosis—symmetrical sensorineural hearing loss with normal impedancemetry.
Shape of the audiometric curve: slope on high frequencies.
Hearing loss can be associated with tinnitus.
Thresholds value for the patient to be considered affected are reported in Table 2 herebelow. These are calculated on the mean of hearing thresholds in bone conduction on 500, 1000, 2000 et 4000 Hz (function of age and gender) on the best ear.

2. Genetic Criteria:
The patient must have at least 1 brother and/or sister affected +/− his children. Father or mother can be affected but not both.

Non inclusion criteria were the following:
Neurological disease at the origin of the hearing loss
Syndromic deafness
Diabetes mellitus treated by oral antidiabetics or insuline
Bilateral chronic otitis, bilateral cholesteatoma
Vestibular schwannoma
Noise induced hearing loss
Ischemic cardiovascular disease

Table 2 shows the thresholds value for the patient to be considered affected. The values correspond to the mean of hearing thresholds in bone conduction on 500, 1000, 2000 and 4000 Hz (function of age and gender). The hearing threshold of affected patients are increased above the values observed in the 25% of the individuals of the same age and gender. Only individuals older than 40 years of age at the time of the diagnosis have been included in the study. (European standard AFNOR 7029:2000-08).

TABLE 2

| MALE | | | FEMALE | |
|---|---|---|---|---|
| | Age | Mean: 0.5, 1, 2, 4 | Age | Mean 0.5, 1, 2, 4 |
| | (years) | KHz | (years) | KHz |
| | 40 | 9 | 40 | 8 |
| | 41 | 10 | 41 | 8 |
| | 42 | 10 | 42 | 9 |
| | 43 | 11 | 43 | 9 |
| | 44 | 11 | 44 | 9 |
| | 45 | 12 | 45 | 10 |
| | 46 | 12 | 46 | 10 |
| | 47 | 13 | 47 | 11 |
| | 48 | 14 | 48 | 11 |
| | 49 | 14 | 49 | 12 |
| | 50 | 15 | 50 | 12 |
| | 51 | 15 | 51 | 12 |
| | 52 | 16 | 52 | 13 |
| | 53 | 17 | 53 | 13 |
| | 54 | 18 | 54 | 14 |
| | 55 | 18 | 55 | 15 |
| | 56 | 19 | 56 | 15 |
| | 57 | 20 | 57 | 16 |
| | 58 | 21 | 58 | 16 |
| | 59 | 21 | 59 | 17 |
| | 60 | 22 | 60 | 17 |
| | 61 | 23 | 61 | 18 |
| | 62 | 24 | 62 | 19 |
| | 63 | 25 | 63 | 19 |
| | 64 | 26 | 64 | 20 |
| | 65 | 27 | 65 | 21 |
| | 66 | 28 | 66 | 21 |
| | 67 | 28 | 67 | 22 |
| | 68 | 29 | 68 | 23 |
| | 69 | 30 | 69 | 24 |
| | 70 | 32 | 70 | 24 |
| | 71 | 33 | 71 | 25 |
| | 72 | 34 | 72 | 26 |
| | 73 | 35 | 73 | 27 |
| | 74 | 36 | 74 | 27 |
| | 75 | 37 | 75 | 28 |
| | 76 | 38 | 76 | 29 |
| | 77 | 39 | 77 | 30 |
| | 78 | 40 | 78 | 31 |
| | 79 | 42 | 79 | 32 |
| | 80 | 43 | 80 | 33 |

### Example 2

### Linkage Analysis

The lod score is a statistical test used for linkage analysis. A lod score greater than 3.0 means that the likelihood of linkage between two allelic markers or a trait and a marker is 1000 times greater than no linkage.

The linkage analysis was carried out using DNA samples collected from 85 families (180 individuals).

DNA was genotyped with the over 300,000 single nucleotide polymorphisms (SNPs or markers) using the Illumina Human Hap300 SNP array. Genotype data were subjected to standard QC procedures used at the CNG (National Genotyping Centre), and samples with less than 95% completion rate (less than 95% of successfully genotyped markers) were eliminated from further consideration. Similarly, markers with less than 95% success rate across the remaining samples, or that showed extreme Hardy-Weinberg distortion were eliminated from further analysis. Sex and pedigree relationships were verified against the data recorded in the medical files established.

For linkage analysis, a subset of markers was chosen that showed low pairwise linkage disequilibrium (LD) in order to eliminate or reduce the upwards bias in lod scores that can be produced by marker LD when not all parents have been genotyped. Non-parametric and parametric affected-only lod scores were calculated with the subset of markers with the recorded sex/pedigree data and the recorded affected status using standard methods (as implemented in the program MERLIN). Parametric affected-only lod scores were calculated under recessive and dominant models with incomplete penetrance.

In the two instances in which lod scores >3 were detected with the non-parametric and dominant affected-only calculations, the 1-lod unit intervals was used to define the region likely to contain the putative susceptibility loci. These chromosomal regions were evaluated for association, taking into account the linkage results, by using standard methods (as implemented in the program LAMP).

### Example 3

### Results

A lod score of 3.4 was found for the chromosomal region located between 187910000 and 189120000 of human chromosome 3 and comprising three mutations as indicated in table 3 below.

TABLE 3

| | | Allele most | Location | |
|---|---|---|---|---|
| Position | | frequently | within the | Mutation in the |
| on SEQ | Alternative | found in affected | adiponectin | corresponding |
| ID NO: 1 | alleles | individuals | gene | protein |
| 4988 | T/C | C | 5′ UTR | n/a |
| 15361 | T/A | A | Coding | Leu9Gln |
| | | | sequence | |
| 16514 | G/A | A | Coding | Gly90Ser |
| | | | sequence | |

The Gly90Ser mutation is known to cause impaired multimerization and/or the consequent impaired secretion of adiponectin (Waki et al. J Biol. Chem. 2003 Oct. 10; 278(41):40352-63). The Leu9Gln mutation is located in the signal peptide, and may thus lead to impaired secretion of adiponectin as well. Therefore, it is believed that reduced levels of adiponectin cause presbycusis. Presbycusis may thus be treated by administering adiponectin or an agonist thereof. Alternatively, presbycusis may be treated by administering an agonist of ADIPOR2, which is one of the receptors of adiponectin.

### Example 4

### Genetic Analysis

Additional samples taken from familial cases of presbycusis were analyzed. The samples were taken from patients enrolled based on the same criteria as those set forth in Example 1.

A total of 113 patients were screened for mutations in the 3 coding exons of the adiponectin gene (ADIPOQ). The primers are shown in the table below.

| | | | SEQ |
|---|---|---|---|
| | | | ID |
| Exon | sense | séquence | NO: |
| 1 | forward | TTTGACTTTTGCCCCATCTTC | 12 |
| | reverse | TGCATATCTGCCTCCACCAG | 13 |
| 2 | forward | GGGCAGAAAGAGATCAAGGTG | 14 |
| | reverse | TGGAAGACCAACCCCAAATC | 15 |
| 3 | forward | CTATAAGTCAAGAAGGTTGTGAGTGG | 16 |
| | reverse | ATCTGGTCATGTTTGTGAAGCTC | 17 |

No mutation was detected in exon 1.

Twenty-one of the 113 patients had a silent polymorphism in exon 2 (15380T>G).

Ten other patients had at least one mutation leading to the presence of a mutation in the adiponectin protein. These mutations were located in exon 3. One of these ten patients carried the Gly90Ser (16514G>A) mutation already detected in the first batch of patients (see Example 3). Nine patients of these ten patients carried a Tyr111His (16577 T>C) mutation. Those two mutations (Gly90Ser and Tyr111His) have been already described in diabetic patients. Among the nine patients carrying the Tyr111His mutation, one patient had a second mutation, Tyr167Cys (16746A>G), which had not been described until now.

These results confirm the results presented in Examples 2 and 3, and show the link between impaired adiponectin function and hearing loss.

Two of the mutations identified in the frame of the present study (Gly90Ser and Tyr111His) had already been described in patients suffering from type II diabetes without obesity. However, the patients enrolled in this study have not been diagnosed with type II diabetes. The recruitment of patients based on hearing loss allows identifying a population of patients without diagnosed type II diabetes, but suffering from presbycusis.

The link between impaired adiponectin function and hearing loss is a major finding, which leads to many applications in diagnosis, patient monitoring and therapeutic treatment of hearing loss, and more specifically presbycusis.

### Example 5

### Physiologic Analysis

Studies have been conducted in order to determine the presence of adiponectin in cells involved in hearing. In mice, adiponectin was found to be present in auditory sensory cells, more specifically in inner and outer ciliated sensory cells (FIGS. 1A and 1B). This result was confirmed using three different antibodies recognizing different epitopes of adiponectin. In addition, the adiponectin receptor protein 1 (ADIPOR1) was also found to be present in mouse auditory sensory cells (FIG. 1C). Indeed, ADIPOR1 was found to be present at the top of the ciliary tuft of inner and outer ciliated sensory cells. Ciliary tuft is the cellular compartment in which the mecano-electric transduction takes place. It was further shown that the survival pathway that involves the binding of adiponectin to ADIPOR1 was present in mouse auditory sensory cells.

Taken together, these results demonstrate that adiponectin plays a major role in auditory sensory cells, in particular in the survival of these cells.

1. An isolated, purified or recombinant adiponectin polynucleotide comprising a sequence at least 80% identical to SEQ ID NO: 1 or a fragment of at least 10 nucleotides of said sequence, characterized in that said polynucleotide comprises:
a) a cytosine at position 4988 of SEQ ID NO: 1;
b) an adenosine at position 15361 of SEQ ID NO: 1;
c) a guanosine at position 15380 of SEQ ID NO: 1; and/or
d) a guanosine at position 16746 of SEQ ID NO: 1.

2. An isolated, purified or recombinant adiponectin polypeptide comprising a sequence at least 80% identical to SEQ ID NO: 2 or a fragment of at least 10 amino acids of said sequence, characterized in that said polypeptide comprises a glutamine at amino acid position 9 of SEQ ID NO: 2 and/or a cysteine at position 167 of SEQ ID NO: 2.

3. Use of at least one adiponectin-related allelic marker for determining whether there is a genetic association between said allelic marker and presbycusis.

4. Use of at least one adiponectin-related allelic marker for diagnosing whether an individual suffers from or is at risk of suffering from presbycusis.

5. Use according to claim 3 or 4, wherein said adiponectin-related allelic marker is selected from the group consisting of the adiponectin-related allelic markers shown in the table below:

| | Position on SEQ ID | |
|---|---|---|
| Allelic marker No. | NO: 1 | Alternative alleles |
| 1 | 4988 | T/C |
| 2 | 15361 | T/A |
| 3 | 16514 | G/A |
| 4 | 15380 | T/G |
| 5 | 16577 | T/C |
| 6 | 16746 | A/G |

6. A method of genotyping comprising the steps of:
a) obtaining an isolated nucleic acid from a biological sample derived from a single individual; and
b) detecting the nucleotide present at one or more of the adiponectin-related allelic markers shown in the table set forth in claim 5.

7. The method of claim 6, further comprising the step of correlating the result of the genotyping steps with a risk of suffering from presbycusis and/or diagnosis of presbycusis.

8. The method of claim 7, wherein the presence of:
a) a C at position 4988 of SEQ ID NO: 1;
b) an A at position 15361 of SEQ ID NO: 1;
c) an A at position 16514 of SEQ ID NO: 1;
d) a G at position 15380 of SEQ ID NO: 1;
e) a C at position 16577 of SEQ ID NO: 1; and/or
f) a G at position 16746 of SEQ ID NO: 1;
indicates that said individual suffers from or is at risk of suffering from presbycusis.

9. An isolated, purified or recombinant probe comprising a fragment of at least 12 nucleotides of an adiponectin gene, wherein said probe comprises a cytosine at position 4988 of SEQ ID NO: 1, an adenosine at position 15361 of SEQ ID NO: 1, a guanosine at position 15380 of SEQ ID NO: 1 and/or a guanosine at position 16746 of SEQ ID NO: 1.

10. The probe of claim 9, wherein said fragment consists of a contiguous span of at least 12 nucleotides of SEQ ID NO: 1.

11. Use of an isolated, purified or recombinant primer comprising a fragment of at least 12 nucleotides of an adiponectin gene for determining the identity of the nucleotide present at least one adiponectin-related allelic marker shown in the table set forth in claim 5.

12. The use of claim 11, wherein the 3′ end of said primer is located within 500 nucleotides of allelic marker NO: 1, 2, 4 or 6 of the table set forth in claim 5.

13. The use of claim 11 or 12, wherein said fragment consists of a contiguous span of at least 12 nucleotides of SEQ ID NO: 1.

14. A primer as defined in any of claims 11 to 13, wherein the 3′ end of said polynucleotide is located:
i) at said biallelic marker; or
ii) one nucleotide upstream of said allelic marker.

15. A pair of primers comprising a first and a second primer each comprising a fragment of an adiponectin gene, wherein:
a) said first primer hybridizes to a first DNA strand of said adiponectin gene;
b) said second primer hybridizes to the strand complementary to said first DNA strand; and
c) the 3′ ends of said first and second primers are located in the vicinity of allelic marker NO: 1, 2, 4 or 6 of the table set forth in claim 5.

16. The pair of primers of claim 15, wherein:
a) said first primer comprises a contiguous span of at least 12 nucleotides of SEQ ID NO: 1; and
b) a second primer comprises a contiguous span of at least 12 nucleotides of a sequence complementary to SEQ ID NO: 1.

17. The pair of primers of claim 16, wherein said pair of primers consists of:
i) SEQ ID NOs: 4 and 5;
ii) SEQ ID NOs: 6 and 7;
iii) SEQ ID NOs: 12 and 13;
iv) SEQ ID NOs: 14 and 15; or
v) SEQ ID NOs: 16 and 17.

18. A method of identifying an ADIPOR2-related allelic marker likely to be associated with presbycusis comprising the steps of:
a) providing biological samples from at least one individual suffering from presbycusis and from at least one unaffected individual;
b) determining the sequence of an ADIPOR2 gene or a fragment thereof present in said samples; and
c) comparing said sequences;
wherein a difference in said sequences is identified as an ADIPOR2-related allelic marker.

19. The method of claim 18, further comprising the step of determining whether there is a genetic association between said ADIPOR2-related allelic marker and presbycusis.

20. An adiponectin polynucleotide for use in the treatment of presbycusis.

21. An adiponectin polypeptide, a fragment of at least 10 amino acids thereof, or an agonist thereof for use in the treatment of presbycusis.

22. An ADIPOR2 agonist for use in the treatment of presbycusis.

23. A method of determining whether an individual suffers from or is at risk of suffering from presbycusis comprising the steps of:
a) providing a biological sample from said individual; and
b) comparing:
ii. the amount of adiponectin polypeptide, or of an RNA species encoding said polypeptide, within said biological sample with an amount detected in or expected from a control sample; or
iii. the activity of adiponectin polypeptide within said biological sample with an activity detected in or expected from a control sample;
wherein a reduced amount or activity of said adiponectin polypeptide or said RNA species within said biological sample compared to said level detected in or expected from said control sample indicates that said individual suffers from or is at risk of suffering from presbycusis.
